SEQUENCE LISTING (1 sequence)

SEQ ID NO: 1 - protein, 458 aa, Homo sapiens
  MISC_FEATURE (5)..(458): Amino Acid Sequence residues 13-466 of human Glucokinase
MKLMALTLVEQILAEFQLQEEDLKKVMRRMQKEMDRGLRLETHEEASVKMLPTYVRSTPEGSEVGDFLSLDLGGTNFRVMLVKVGEGEEGQWSVKTKHQMYSIPEDAMTGTAEMLFDYISECISDFLDKHQMKHKKLPLGFTFSFPVRHEDIDKGILLNWTKGFKASGAEGNNVVGLLRDAIKRRGDFEMDVVAMVNDTVATMISCYYEDHQCEVGMIVGTGCNACYMEEMQNVELVEGDEGRMCVNTEWGAFGDSGELDEFLLEYDRLVDESSANPGQQLYEKLIGGKYMGELVRLVLLRLVDENLLFHGEASEQLRTRGAFETRFVSQVESDTGDRKQIYNILSTLGLRPSTTDCDIVRRACESVSTRAAHMCSAGLAGVINRMRESRSEDVMRITVGVDGSVYKLHPSFKERFHASVRRLTPSCEITFIESEEGSGRGAALVSAVACKKACMLGQ

(19) United States
(12) Patent Application Publication
(10) Pub. No.: US 2007/0197532 A1
(43) Pub. Date: Aug. 23, 2007

(54) GLUCOKINASE ACTIVATORS

(76) Inventors: Sheldon X. Cao, San Diego, CA (US); Jun Feng, Carlsbad, CA (US); Yasuhiro Imaeda, Osaka (JP); Stephen L. Gwaltney, San Diego, CA (US); David J. Hosfield, Solana Beach, CA (US); Nobuyuki Takakura, Osaka (JP); Mingnam Tang, San Diego, CA (US)

Correspondence Address: TAKEDA SAN DIEGO, INC., 10410 SCIENCE CENTER DRIVE, SAN DIEGO, CA, 92121, US

(21) Appl. No.: 11/561,307

(22) Filed: Nov. 17, 2006

Related U.S. Application Data
(60) Provisional application No. 60/738,350, filed on Nov. 18, 2005.

(51) Int. Cl. A61K 31/5377; A61K 31/506; A61K 31/4439; A61K 31/4196; A61K 31/4152; C07D 413/02; C07D 403/02

(52) U.S. Cl. 514/235.5; 514/383; 514/406; 548/371.4; 548/268.4; 514/341; 514/275; 544/132; 544/140; 544/331

(57) ABSTRACT

Compounds, pharmaceutical compositions, kits and methods are provided for use with glucokinase that comprise a compound selected from the group consisting of: 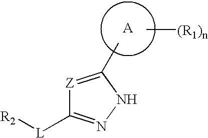 wherein the variables are as defined herein.

### RELATED APPLICATION

This application claims the benefit of U.S. Provisional Application No. 60/738,350, filed Nov. 18, 2005, which is incorporated herein by reference.

### FIELD OF THE INVENTION

The present invention relates to compounds that may be used to activate hexokinases, as well as compositions of matter and kits comprising these compounds. The invention also relates to methods for activating hexokinases and treatment methods using compounds according to the present invention. In particular, the present invention relates to glucokinase activators, compositions of matter and kits comprising these compounds and methods for activating glucokinase.

### BACKGROUND OF THE INVENTION

Glucokinase (GK, Hexokinase IV) is one of four hexokinases that are found in mammals (Colowick, S. P., in The Enzymes, Vol. 9 (P. Boyer, ed.) Academic Press, New York, N.Y., pages 1-48, 1973). The hexokinases catalyze the first step in the metabolism of glucose, i.e., the conversion of glucose to glucose-6-phosphate. Glucokinase is found principally in pancreatic β-cells and liver parenchymal cells, two cell types that are known to play critical roles in whole-body glucose homeostasis. Specifically, GK is a rate-controlling enzyme for glucose metabolism in these two cell types (Chipkin, S. R., Kelly, K. L., and Ruderman, N. B. in Joslin's Diabetes (C. R. Khan and G. C. Wier, eds.), Lea and Febiger, Philadelphia, Pa., pages 97-115, 1994).

The concentration of glucose at which GK demonstrates half-maximal activity is approximately 8 mM. The other three hexokinases are saturated with glucose at much lower concentrations (<1 mM). Therefore, the flux of glucose through the GK pathway rises as the concentration of glucose in the blood increases from fasting levels (5 mM) to postprandial levels following a carbohydrate-containing meal (about 10-15 mM) (Printz, R. G., Magnuson, M. A., and Granner, D. K. in Ann. Rev. Nutrition Vol. 13 (R. E. Olson, D. M. Bier, and D. B. McCormick, eds.), Annual Review, Inc., Palo Alto, Calif., pages 463-496, 1993). These findings suggest that GK functions as a glucose sensor in β-cells and hepatocytes (Meglasson, M. D. and Matschinsky, F. M. Amer. J Physiol. 246, E1-E13, 1984).

More recently, studies in transgenic animals confirmed that GK does indeed play a critical role in whole-body glucose homeostasis. Animals that do not express GK die within days of birth with severe diabetes, while animals overexpressing GK have improved glucose tolerance (Grupe, A., Hultgren, B., Ryan, A. et al., Cell 83, 69-78, 1995; Ferrie, T., Riu, E., Bosch, F. et al., FASEB J., 10, 1213-1218, 1996). An increase in glucose exposure is coupled through GK in β-cells to increased insulin secretion and in hepatocytes to increased glycogen deposition and perhaps decreased glucose production.

The finding that type II maturity-onset diabetes of the young (MODY-2) is caused by loss of function mutations in the GK gene suggests that GK also functions as a glucose sensor in humans (Liang, Y., Kesavan, P., Wang, L. et al., Biochem. J. 309, 167-173, 1995). Additional evidence supporting an important role for GK in the regulation of glucose metabolism in humans was provided by the identification of patients that express a mutant form of GK with increased enzymatic activity. These patients exhibit a fasting hypoglycemia associated with an inappropriately elevated level of plasma insulin (Glaser, B., Kesavan, P., Heyman, M. et al., New England J. Med. 338, 226-230, 1998). Accordingly, compounds that activate GK and, thereby, increase the sensitivity of the GK sensor system are expected to be useful in the treatment of the hyperglycemia characteristic of all type II diabetes. Glucokinase activators should increase the flux of glucose metabolism in β-cells and hepatocytes, which will be coupled to increased insulin secretion.

There is a continued need to find new therapeutic agents to treat human diseases. The hexokinases, specifically but not limited to glucokinase, are especially attractive targets for the discovery of new therapeutics due to their important role in diabetes, hyperglycemia and other diseases.

### SUMMARY OF THE INVENTION

The present invention relates to compounds that activate glucokinase. The present invention also provides compositions, articles of manufacture and kits comprising these compounds.

In one embodiment, a pharmaceutical composition is provided that comprises a glucokinase activator according to the present invention as an active ingredient. Pharmaceutical compositions according to the invention may optionally comprise 0.001%-100% of one or more activators of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

The invention is also directed to kits and other articles of manufacture for treating disease states associated with glucokinase.

In one embodiment, a kit is provided that comprises a composition comprising at least one glucokinase activator of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one glucokinase activator of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

Also provided are methods for preparing compounds, compositions and kits according to the present invention. For example, several synthetic schemes are provided herein for synthesizing compounds according to the present invention.

Also provided are methods for using compounds, compositions, kits and articles of manufacture according to the present invention.

In one embodiment, the compounds, compositions, kits and articles of manufacture are used to modulate glucokinase. In particular, the compounds, compositions, kits and articles of manufacture can be used to activate glucokinase.

In another embodiment, the compounds, compositions, kits and articles of manufacture are used to treat a disease state for which increasing glucokinase activity ameliorates the pathology and/or symptomology of the disease state.

In another embodiment, a compound is administered to a subject wherein glucokinase activity within the subject is altered and, in one embodiment, increased.

In another embodiment, a prodrug of a compound is administered to a subject that is converted to the compound in vivo where it activates glucokinase.

In another embodiment, a method of activating glucokinase is provided that comprises contacting glucokinase with a compound according to the present invention.

In another embodiment, a method of activating glucokinase is provided that comprises causing a compound according to the present invention to be present in a subject in order to activate glucokinase in vivo.

In another embodiment, a method of activating glucokinase is provided that comprises administering a first compound to a subject that is converted in vivo to a second compound wherein the second compound activates glucokinase in vivo. It is noted that the compounds of the present invention may be the first or second compounds.

In another embodiment, a therapeutic method is provided that comprises administering a compound according to the present invention.

In another embodiment, a method of treating a condition in a patient that is known to be mediated by glucokinase, or which is known to be treated by glucokinase activators, is provided comprising administering to the patient a therapeutically effective amount of a compound according to the present invention.

In another embodiment, a method is provided for treating a disease state for which increasing glucokinase activity ameliorates the pathology and/or symptomology of the disease state, the method comprising: causing a compound according to the present invention to be present in a subject in a therapeutically effective amount for the disease state.

In another embodiment, a method is provided for treating a disease state for which increasing glucokinase activity ameliorates the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which increasing glucokinase activity ameliorates the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by glucokinase, or that is known to be treated by glucokinase activators.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well know in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to activating glucokinase and thus may themselves be prodrugs for another compound. Such prodrugs of another compound may or may not themselves independently have glucokinase activity.

### BRIEF DESCRIPTION OF THE FIGURES

FIG. 1 illustrates SEQ. ID No. 1 referred to in this application.

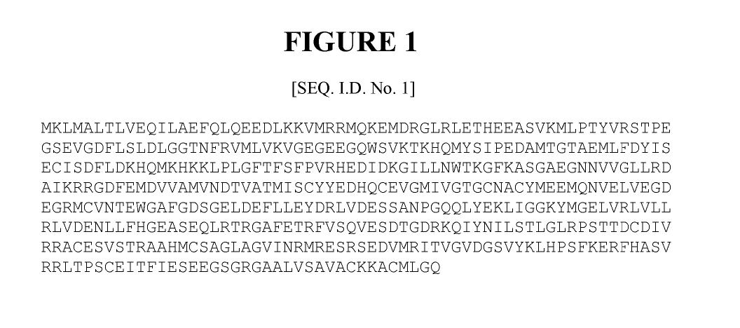

### DEFINITIONS

Unless otherwise stated, the following terms used in the specification and claims shall have the following meanings for the purposes of this application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃₋₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with oxygen (See “oxaalkyl”) or nitrogen atoms (See “aminoalkyl”) between the carbon atoms. C_{X} alkyl and C_{X-Y} alkyl are typically used where X and Y indicate the number of carbon atoms in the chain. For example, C₁₋₆ alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenethyl, 1-phenylethyl, 3-phenylpropyl, 2-thienylmethyl, 2-pyridinylmethyl and the like).

“Alkenyl” means a straight or branched, carbon chain that contains at least one carbon-carbon double bond. Examples of alkenyl include vinyl, allyl, isopropenyl, pentenyl, hexenyl, heptenyl, 1-propenyl, 2-butenyl, 2-methyl-2-butenyl, and the like.

“Alkynyl” means a straight or branched, carbon chain that contains at least one carbon-carbon triple bond. Examples of alkynyl include ethynyl, propargyl, 3-methyl-1-pentynyl, 2-heptynyl and the like.

“Alkylene”, unless indicated otherwise, means a straight or branched, saturated or unsaturated, aliphatic, divalent radical. C_{X} alkylene and C_{X-Y} alkylene are typically used where X and Y indicate the number of carbon atoms in the chain. For example, C₁₋₆ alkylene includes methylene (—CH₂—), ethylene (—CH₂CH₂—), trimethylene (—CH₂CH₂CH₂—), tetramethylene (—CH₂CH₂CH₂CH₂—) 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like.

“Alkenylene” means a straight or branched, divalent carbon chain having one or more carbon-carbon double bonds. Examples of alkenylene include ethene-1,2-diyl, propene-1,3-diyl, methylene-1,1-diyl, and the like.

“Alkynylene” means a straight or branched, divalent carbon chain having one or more carbon-carbon triple bonds. Examples of alkynylene include ethyne-1,2-diyl, propyne-1,3-diyl, and the like.

“Alkylidene” means a straight or branched saturated or unsaturated, aliphatic radical connected to the parent molecule by a double bond. C_{X} alkylidene and C_{X-Y} alkylidene are typically used where X and Y indicate the number of carbon atoms in the chain. For example, C₁₋₆ alkylidene includes methylene (═CH₂), ethylidene (═CHCH₃), isopropylidene (═C(CH₃)₂), propylidene (═CHCH₂CH₃), allylidene (═CH—CH═CH₂), and the like).

“Amino” means a nitrogen moiety having two further substituents where, for example, a hydrogen or carbon atom is attached to the nitrogen. For example, representative amino groups include —NH₂, —NHCH₃, —N(CH₃)₂, —NHC₁₋₁₀-alkyl, —N(C₁₋₁₀-alkyl)₂, —NHaryl, —NHheteroaryl, —N(aryl)₂, —N(heteroaryl)₂, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Aminoalkyl” means an alkyl, as defined above, except where one or more substituted or unsubstituted nitrogen atoms (—N—) are positioned between carbon atoms of the alkyl. For example, an (C₂₋₆) aminoalkyl refers to a chain comprising between 2 and 6 carbons and one or more nitrogen atoms positioned between the carbon atoms.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (see Heteroaryl).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. C_{X} aryl and C_{X-Y} aryl are typically used where X and Y indicate the number of atoms in the ring.

“Bicycloalkyl” means a saturated or partially unsaturated fused bicyclic or bridged polycyclic ring assembly.

“Bicycloaryl” means a bicyclic ring assembly wherein the rings are linked by a single bond or fused and at least one of the rings comprising the assembly is aromatic. C_{X} bicycloaryl and C_{X-Y} bicycloaryl are typically used where X and Y indicate the number of carbon atoms in the bicyclic ring assembly and directly attached to the ring.

“Bridging ring” as used herein refers to a ring that is bonded to another ring to form a compound having a bicyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRₓR_{y} where Rₓ and R_{y} are each independently two further substituents where a hydrogen or carbon atom is attached to the nitrogen.

“Carbocycle” means a ring consisting of carbon atoms.

“Carbocyclic ketone derivative” means a carbocyclic derivative wherein the ring contains a —CO— moiety.

“Carbonyl” means the radical —CO—. It is noted that the carbonyl radical may be further substituted with a variety of substituents to form different carbonyl groups including acids, acid halides, aldehydes, amides, esters, and ketones.

“Carboxy” means the radical —CO₂—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a non-aromatic, saturated or partially unsaturated, monocyclic, fused bicyclic or bridged polycyclic ring assembly. C_{X} cycloalkyl and C_{X-Y} cycloalkyl are typically used where X and Y indicate the number of carbon atoms in the ring assembly. For example, C₃₋₁₀ cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

“Cycloalkylene” means a divalent saturated or partially unsaturated, monocyclic or polycyclic ring assembly. C_{X} cycloalkylene and C_{X-Y} cycloalkylene are typically used where X and Y indicate the number of carbon atoms in the ring assembly.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated, partially saturated, carbocyclics, heterocyclics, aromatics, heteroaromatics, and the like.

“Halo” means fluoro, chloro, bromo or iodo.

“Halo-substituted alkyl”, as an isolated group or part of a larger group, means “alkyl” substituted by one or more “halo” atoms, as such terms are defined in this application. Halo-substituted alkyl includes haloalkyl, dihaloalkyl, trihaloalkyl, perhaloalkyl and the like (e.g., halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroatom” refers to an atom that is not a carbon atom. Particular examples of heteroatoms include, but are not limited to nitrogen, oxygen, and sulfur.

“Heteroatom moiety” includes a moiety where the atom by which the moiety is attached is not a carbon. Examples of heteroatom moieties include —N═, —NR_{c}—, —N⁻(O⁻)═, —O—, —S— or —S(O)₂—, wherein R_{c} is further substituent.

“Heterobicycloalkyl” means bicycloalkyl, as defined in this application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like.

“Heterocycloalkylene” means cycloalkylene, as defined in this application, provided that one or more of the ring member carbon atoms is replaced by a heteroatom.

“Heteroaryl” means a cyclic aromatic group having five or six ring atoms, wherein at least one ring atom is a heteroatom and the remaining ring atoms are carbon. The nitrogen atoms can be optionally quaternerized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted.

“Heterobicycloaryl” means bicycloaryl, as defined in this application, provided that one or more of the atoms within the ring is a heteroatom. For example, hetero(C₄₋₁₂)bicycloaryl as used in this application includes, but is not limited to, 2-amino-4-oxo-3,4-dihydropteridin-6-yl, tetrahydroisoquinolinyl, and the like.

“Heterocycloalkyl” means cycloalkyl, as defined in this application, provided that one or more of the atoms forming the ring is a heteroatom selected, independently from N, O, or S. Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

“Hydroxy” means the radical —OH.

“Iminoketone derivative” means a derivative comprising the moiety —C(NR)—, wherein R comprises a hydrogen or carbon atom attached to the nitrogen.

“Isomers” mean any compound having an identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four nonidentical substituents is termed a “chiral center.” A compound with one chiral center has two enantiomeric forms of opposite chirality. A mixture of the two enantiomeric forms is termed a “racemic mixture.” A compound that has more than one chiral center has 2ⁿ⁻¹enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Nitro” means the radical —NO₂.

“Oxaalkyl” means an alkyl, as defined above, except where one or more oxygen atoms (—O—) are positioned between carbon atoms of the alkyl. For example, an (C₂₋₆)oxaalkyl refers to a chain comprising between 2 and 6 carbons and one or more oxygen atoms positioned between the carbon atoms.

“Oxoalkyl” means an alkyl, further substituted with a carbonyl group. The carbonyl group may be an aldehyde, ketone, ester, amide, acid or acid chloride.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound that is convertible in vivo metabolically into an activator according to the present invention. The prodrug itself may or may not also have glucokinase activity. For example, an activator comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, an activator comprising an amine group may be administered as an amide that is converted by hydrolysis in vivo to the amine compound.

“Protected derivatives” means derivatives of activators in which a reactive site or sites are blocked with protecting groups. Protected derivatives are useful in the preparation of activators or in themselves may be active. A comprehensive list of suitable protecting groups can be found in T. W. Greene, Protecting Groups in Organic Synthesis, 3rd edition, John Wiley & Sons, Inc. 1999.

“Ring” means a carbocyclic or a heterocyclic system.

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —SO—. It is noted that the sulfinyl radical may be further substituted with a variety of substituents to form different sulfinyl groups including sulfinic acids, sulfinamides, sulfinyl esters, and sulfoxides.

“Sulfonyl” means the radical —SO₂—. It is noted that the sulfonyl radical may be further substituted with a variety of substituents to form different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thiocarbonyl” means the radical —CS—. It is noted that the thiocarbonyl radical may be further substituted with a variety of substituents to form different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₓR_{y}R_{z} where Rₓ, R_{y}, and R_{z} may each independently be hydrogen or any other substituent where the atom attached to the carbon is a heteroatom or cyano. Hence, CF₃, CH₂OH and CH₂CN, for example, are all C₁ alkyls.

### DETAILED DESCRIPTION OF THE INVENTION

The present invention relates to compounds, compositions, kits and articles of manufacture that may be used to activate glucokinase. It is noted that the compounds of the present invention may also possess activity for other hexokinases family members and thus may be used to address disease states associated with these other family members.

Glucokinase Activators

In one of its aspects, the present invention relates to Glucokinase activators. In one embodiment, glucokinase activators of the present invention comprise: 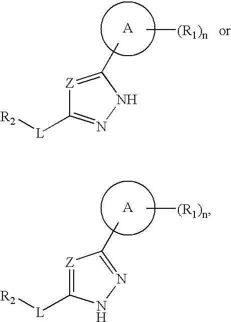
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
Z is selected from the group consisting of CR₃ and N;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
   R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
   R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In another embodiment, glucokinase activators of the present invention comprise:

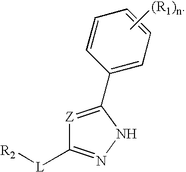

In still another embodiment, glucokinase activators of the present invention comprise:

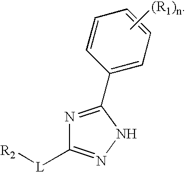

In yet another embodiment, glucokinase activators of the present invention comprise:

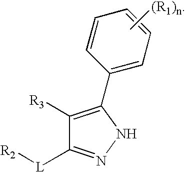

In a further embodiment, glucokinase activators of the present invention comprise:

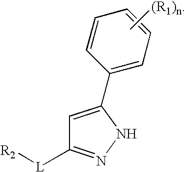

In still a further embodiment, glucokinase activators of the present invention comprise:

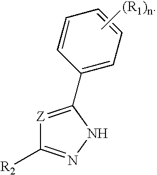

In yet a further embodiment, glucokinase activators of the present invention comprise:

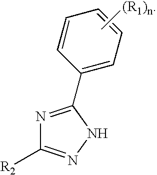

In another embodiment, glucokinase activators of the present invention comprise:

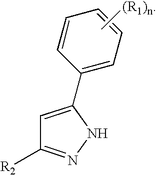

In still another embodiment, glucokinase activators of the present invention comprise: 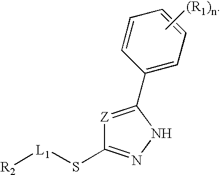
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In yet another embodiment, glucokinase activators of the present invention comprise: 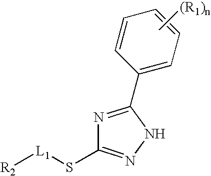
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In a further embodiment, glucokinase activators of the present invention comprise: 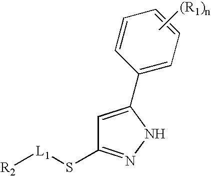
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In still a further embodiment, glucokinase activators of the present invention comprise: 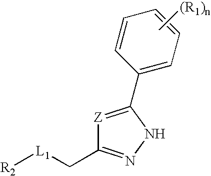
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In yet a further embodiment, glucokinase activators of the present invention comprise: 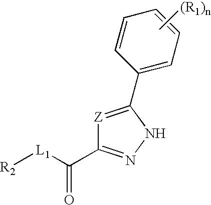
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In another embodiment, glucokinase activators of the present invention comprise: 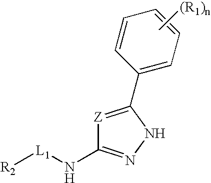
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In still another embodiment, glucokinase activators of the present invention comprise: 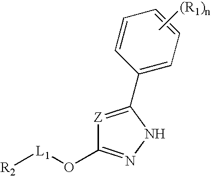
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In yet another embodiment, glucokinase activators of the present invention comprise: 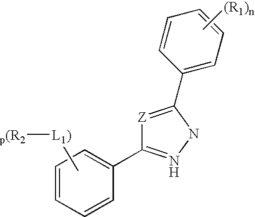
wherein
p is selected from the group consisting of 0, 1, 2, 3, 4 and 5; and
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

In a further embodiment, glucokinase activators of the present invention comprise: 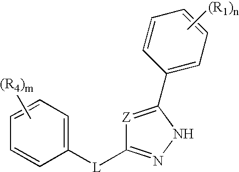
wherein
m is selected from the group consisting of 0, 1, 2, 3, 4 and 5; and
R₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₄ are taken together to form a ring.

In still a further embodiment, glucokinase activators of the present invention comprise: 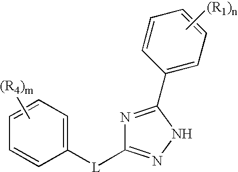
wherein
m is selected from the group consisting of 0, 1, 2, 3, 4 and 5; and
R₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₄ are taken together to form a ring.

In yet a further embodiment, glucokinase activators of the present invention comprise: 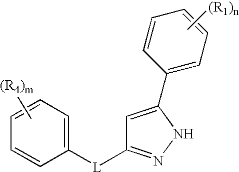
wherein
m is selected from the group consisting of 0, 1, 2, 3, 4 and 5; and
R₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₄ are taken together to form a ring.

In another embodiment, glucokinase activators of the present invention comprise: 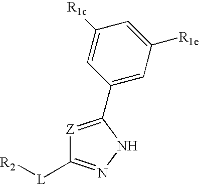
wherein R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still another embodiment, glucokinase activators of the present invention comprise: 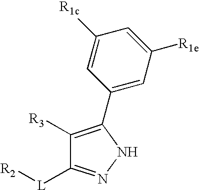
wherein R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet another embodiment, glucokinase activators of the present invention comprise: 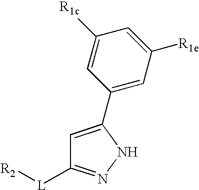
wherein R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In a further embodiment, glucokinase activators of the present invention comprise: 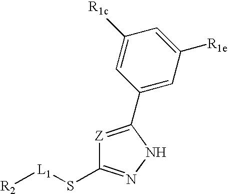
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still a further embodiment, glucokinase activators of the present invention comprise: 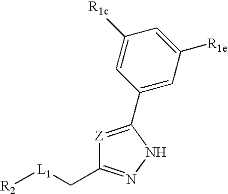
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet a further embodiment, glucokinase activators of the present invention comprise: 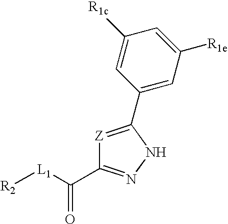
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In another embodiment, glucokinase activators of the present invention comprise: 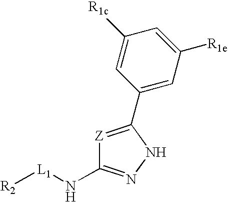
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still another embodiment, glucokinase activators of the present invention comprise: 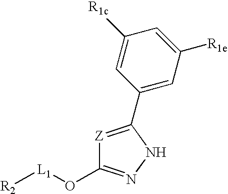
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet another embodiment, glucokinase activators of the present invention comprise: 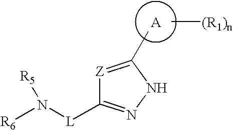
wherein R₅ and R₆ are each independently selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₅ and R₆ are taken together to form a substituted or unsubstituted ring.

In another of its aspects, the present invention relates to processes of making the compounds of the present invention. In one embodiment, the process comprises:

reacting a compound comprising the formula 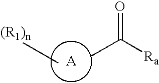
with hydrazinecarbothioamide under conditions that form a first reaction product comprising the formula

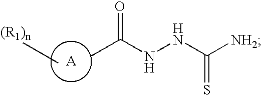

treating the first reaction product under conditions that form a second reaction product comprising the formula

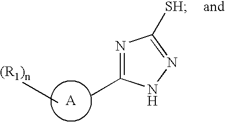

reacting the second reaction product with a compound comprising the formula R₂-L₁-X under conditions that form a product comprising the formula 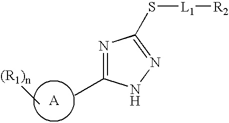
wherein
X and Rₐ are each independently a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In another embodiment, the process comprises:

treating a compound comprising the formula 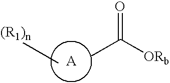
under conditions that form a first reaction product comprising the formula

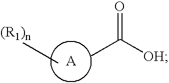

reacting the first reaction product with a compound comprising the formula R_{c}O₂C—C(R₃)H—CO₂K under conditions that form a second reaction product comprising the formula

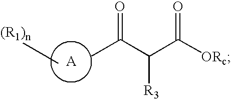

treating the second reaction product under conditions that form a third reaction product comprising the formula

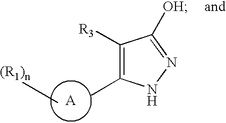

reacting the third reaction product with a compound comprising the formula R₂-L₁-OH under conditions that form a product comprising the formula 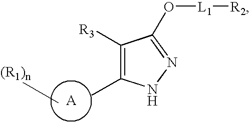
wherein
R_{b} and R_{c} are each independently a (C₁₋₅)alkyl;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still another embodiment, the process comprises:

reacting a compound comprising the formula 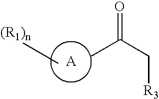
with a compound comprising the formula (CO₂R_{d})₂ under conditions that form a first reaction product comprising the formula

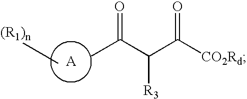

treating the first reaction product under conditions that form a second reaction product comprising the formula

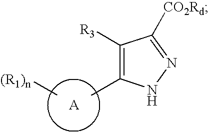

treating the second reaction product under conditions that form a third reaction product comprising the formula

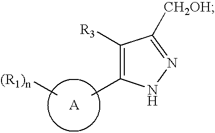

reacting the third reaction product with a compound comprising the formula R₂—OH under conditions that form a product comprising the formula 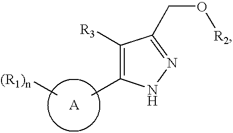
wherein
R_{d} is a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet another embodiment, the process comprises:

reacting a compound comprising the formula 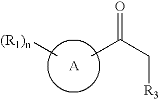
with a compound comprising the formula R₂-L-CO₂Rₑ under conditions that form a first reaction product comprising the formula 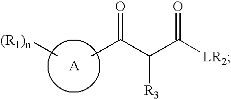
and

treating the first reaction product under conditions that form a product comprising the formula 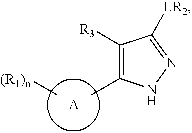
wherein
Rₑ is a (C₁₋₅)alkyl;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In a further embodiment, the process comprises:

reacting a compound comprising the formula 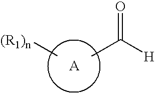
with a compound comprising the formula
R₃ [symbol] LR₂
under conditions that form a product comprising the formula 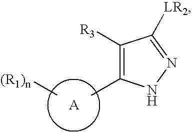
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still a further embodiment, the process comprises:

reacting a compound comprising the formula 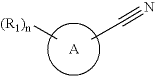
with a compound comprising the formula 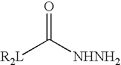
under conditions that form a product comprising the formula 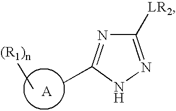
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and

In yet a further embodiment, the process comprises:

treating a compound comprising the formula 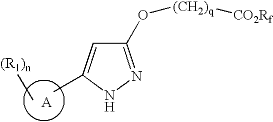
under conditions that form a first reaction product comprising the formula

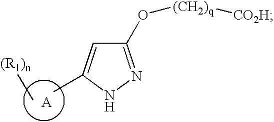

reacting the first reaction product with a compound comprising the formula HNR₅R₆ under conditions that form a product comprising the formula 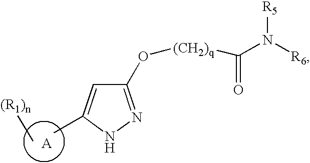
wherein
n is selected from the group consisting of 0, 1, 2, 3, 4 and 5;
q is selected from the group consisting of 0, 1, 2, 3, 4 and 5;
R_{f} is a (C₁₋₅)alkyl;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₅ and R₆ are each independently selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₅ and R₆ are taken together to form a substituted or unsubstituted ring.

In another embodiment, the process comprises:

treating a compound comprising the formula 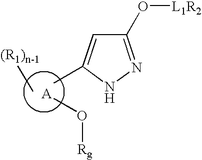
under conditions that form a first reaction product comprising the formula 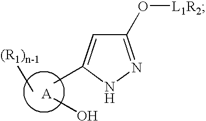
and

reacting the first reaction product with a compound comprising Q-OH under conditions that form a product comprising the formula 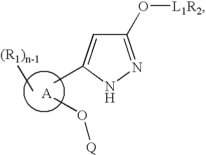
wherein
R_{g} is a leaving group;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still another embodiment, the process comprises:

reacting a compound comprising the formula 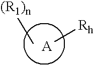
with a compound comprising the formula 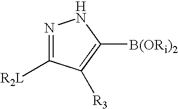
under conditions that form a product comprising the formula 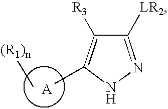
wherein
Rₕ and Rᵢ are each independently a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still another of its aspects, the present invention relates to intermediates useful in making compounds of the present invention. In one embodiment, the intermediate comprises the formula 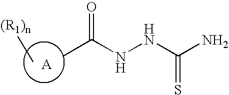
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

In another embodiment, the intermediate comprises the formula 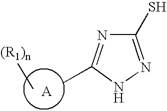
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

In still another embodiment, the intermediate comprises the formula 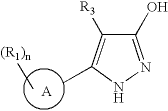
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet another embodiment, the intermediate comprises the formula 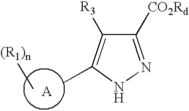
wherein
R_{d} is a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In a further embodiment, the intermediate comprises the formula 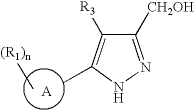
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In still a further embodiment, the intermediate comprises the formula 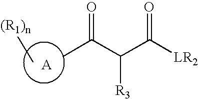
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet a further embodiment, the intermediate comprises the formula 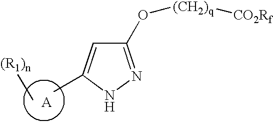
wherein
R_{f} is a (C₁₋₅)alkyl;
n is selected from the group consisting of 0, 1, 2, 3, 4 and 5;
q is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

In another embodiment, the intermediate comprises the formula 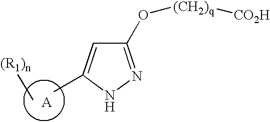
wherein
n is selected from the group consisting of 0, 1, 2, 3, 4 and 5;
q is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

In still another embodiment, the intermediate comprises the formula 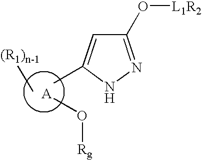
wherein
R_{g} is a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In yet another embodiment, the intermediate comprises the formula 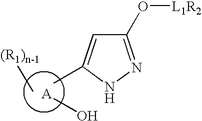
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In a further embodiment, the intermediate comprises the formula 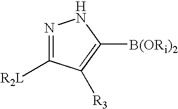
wherein
R₁ is a leaving group;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In one variation of each of the above embodiments, Z is CR₃.

In another variation of each of the above embodiments and variations, m is 2.

In still another variation of each of the above embodiments and variations, n is selected from the group consisting of 1, 2, 3, 4 and 5. In yet another variation of each of the above embodiments and variations, n is 2.

In a further variation of each of the above embodiments and variations, p is selected from the group consisting of 1, 2, 3, 4 and 5. In still a further variation of each of the above embodiments and variations, p is 2.

In yet a further variation of each of the above embodiments and variations, q is selected from the group consisting of 1, 2, 3, 4 and 5. In another variation of each of the above embodiments and variations, q is selected from the group consisting of 1, 2 and 3.

In still another variation of each of the above embodiments and variations, L is absent or L comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted. In yet another variation of each of the above embodiments and variations, L is —O—. In a further variation of each of the above embodiments and variations, L is —O—(CH₂)ₘ—CO— and m is selected from the group consisting of 0, 1, 2, 3, 4, and 5.

In still a further variation of each of the above embodiments and variations, L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted. In yet a further variation of each of the above embodiments and variations, L₁ is —O—.

In another variation of each of the above embodiments and variations, R₁ is selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted. In still another variation of each of the above embodiments and variations, at least one R₁ is alkoxy. In yet another variation of each of the above embodiments and variations, at least one R₁ is methoxy. In a further variation of each of the above embodiments and variations, R₁ is 2-methoxy.

In still a further variation of each of the above embodiments and variations, R₁ is Q-M-; Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

In yet a further variation of each of the above embodiments and variations, R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted. In another variation of each of the above embodiments and variations, R_{1c} and R₁ₑ are each independently a substituted or unsubstituted (C₁₋₅)alkoxy.

In still another variation of each of the above embodiments and variations, R_{1c} and R₁ₑ are each independently Q-M-; Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

In yet another variation of each of the above embodiments and variations, M is absent or M comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted. In a further variation of each of the above embodiments and variations, M is —O—. In still a further variation of each of the above embodiments and variations, M is selected from the group consisting of —CH₂—O— and —O—CH₂—.

In yet a further variation of each of the above embodiments and variations, Q is a substituted or unsubstituted (C₄₋₁₂)aryl. In another variation of each of the above embodiments and variations, Q is a substituted or unsubstituted phenyl. In still another variation of each of the above embodiments and variations, Q is a substituted or unsubstituted hetero(C₁₋₁₀)aryl. In yet another variation of each of the above embodiments and variations, Q is a substituted or unsubstituted pyridinyl. In one particular variation of each of the foregoing variations, each optional substituent is independently selected from the group consisting of (C₁₋₅)alkyl, sulfonyl and (C₁₋₅)alkylsulfonyl.

In a further variation of each of the above embodiments and variations, R₂ is selected from the group consisting of hydrogen, hydroxy, carbonyl, amino, (C₁₋₁₀)alkyl, (C₃₋₁₂)cycloalkyl, heteron (C₃₋₁₂)alkyl, aryl and heteroaryl, each substituted or unsubstituted. In still a further variation of each of the above embodiments and variations, R₂ is a substituted or unsubstituted (C₁₋₅)alkyl. In yet a further variation of each of the above embodiments and variations, R₂ is methyl.

In another variation of each of the above embodiments and variations, R₂ is NR₅R₆; and R₅ and R₆ are each independently selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₅ and R₆ are taken together to form a substituted or unsubstituted ring.

In still another variation of each of the above embodiments and variations, R₃ is H.

In yet another variation of each of the above embodiments and variations, R₄ is selected from the group consisting of halo, nitro, cyano, hydroxy, alkoxy, carbonyl, (C₁₋₁₀)alkyl and halo(C₁₋₁₀)alkyl, each substituted or unsubstituted, or two R₄ are taken together to form a ring.

In a further variation of each of the above embodiments and variations, R₅ is H.

In still a further variation of each of the above embodiments and variations, R₆ is H.

In yet further variations of each of the above embodiments and variations, X, Rₐ and Rₕ are each independently halo.

In yet further variations of each of the above embodiments and variations, R_{b}, R_{c}, R_{d}, Rₑ, R_{f} and Rᵢ are each independently a substituted or unsubstituted (C₁₋₃)alkyl.

In another variation of each of the above embodiments and variations, R_{g} is benzyl.

Particular examples of compounds according to the present invention include, but are not limited to:
5-(2-methoxyphenyl)-3-(methylthio)-1H-1,2,4-triazole;
5-(2-ethoxyphenyl)-3-(2-fluorobenzylthio)-1H-1,2,4-triazole;
5-(2-(benzyloxy)phenyl)-3-(2-fluorobenzylthio)-1H-1,2,4-triazole;
5-(2,4-dimethoxyphenyl)-3-(2-fluorobenzylthio)-1H-1,2,4-triazole;
5-(2,5-dimethoxyphenyl)-3-(2-fluorobenzylthio)-1H-1,2,4-triazole;
2-((5-(2-methoxyphenyl)-1H-1,2,4-triazol-3-ylthio)methyl)pyridine;
3-((5-(2-methoxyphenyl)-1H-1,2,4-triazol-3-ylthio)methyl)pyridine;
2-(5-(2-methoxyphenyl)-1H-1,2,4-triazol-3-ylthio)-N,N-dimethylacetamide;
2-(5-(2-isopropoxyphenyl)-1H-1,2,4-triazol-3-ylthio)-1-morpholinoethanone;
2-(5-(2-isopropoxyphenyl)-1H-1,2,4-triazol-3-ylthio)-N,N-dimethylacetamide;
3-(2-fluorobenzyloxy)-5-(2-methoxyphenyl)-1H-pyrazole;
3-(2-fluorobenzyloxy)-5-phenyl-1H-pyrazole;
3-(2-fluorobenzyloxy)-5-(2-fluorophenyl)-1H-pyrazole;
5-(3,5-dimethoxyphenyl)-3-(2-fluorobenzyloxy)-1H-pyrazole;
3-(2-fluorobenzyloxy)-5-o-tolyl-1H-pyrazole;
5-(2,4-dimethoxyphenyl)-3-(2-fluorobenzyloxy)-1H-pyrazole;
3-((2-fluorophenoxy)methyl)-5-(2-methoxyphenyl)-1H-pyrazole;
5-(2-methoxyphenyl)-3-phenyl-1H-pyrazole;
3-cyclopropyl-5-(2-methoxyphenyl)-1H-pyrazole;
3-(4-chlorophenyl)-5-(2-methoxyphenyl)-1H-pyrazole;
2-(3-phenyl-1H-pyrazol-5-yl)phenol;
4-bromo-2-(3-phenyl-1H-pyrazol-5-yl)phenol;
5-(2,3-dimethoxyphenyl)-3-(2-fluorobenzyloxy)-1H-pyrazole;
5-(2,3-dihydrobenzofuran-7-yl)-3-(2-fluorobenzyloxy)-1H-pyrazole;
5-(5-(benzyloxy)-2-isopropoxyphenyl)-3-(2-fluorobenzyloxy)-1H-pyrazole;
5-(2,5-dimethoxyphenyl)-3-(2-fluorobenzyloxy)-1H-pyrazole;
5-(5-fluoro-2-methoxyphenyl)-3-(2-fluorobenzyloxy)-1H-pyrazole;
5-(2-(difluoromethoxy)phenyl)-3-(2-fluorobenzyloxy)-1H-pyrazole;
2-((2-(3-phenyl-1H-pyrazol-5-yl)phenoxy)methyl)pyridine;
(R)-6-((5-(2-methoxyphenyl)-1H-pyrazol-3-yloxy)methyl)piperazin-2-one;
2-(5-(2-methoxyphenyl)-1H-pyrazol-3-yloxy)acetic acid;
2-(5-(2-methoxyphenyl)-1H-pyrazol-3-yloxy)-1-morpholinoethanone;
(5-(2-methoxyphenyl)-1H-pyrazol-3-yl)(morpholino)methanone;
(5-(2,5-dimethoxyphenyl)-1H-pyrazol-3-yl)(morpholino)methanone;
5-(2-methoxyphenyl)-N-phenyl-1H-pyrazole-3-carboxamide;
3-(4-fluorophenyl)-5-(2-((1-methyl-1H-imidazol-2-yl)methoxy)phenyl)-1H-pyrazole;
(3-(5-(2-methoxyphenyl)-1H-pyrazol-3-yl)phenyl)(morpholino)methanone;
3-(5-(2-methoxyphenyl)-1H-pyrazol-3-yl)benzonitrile;
4-(5-(2-methoxyphenyl)-1H-pyrazol-3-yl)benzonitrile;
3-(4-fluorophenyl)-5-(2-phenoxyphenyl)-1H-pyrazole;
3-(4-fluorophenyl)-5-(3-phenoxyphenyl)-1H-pyrazole;
2-(3-(3-(4-fluorophenyl)-1H-pyrazol-5-yl)phenoxy)pyrimidine;
5-(4-(benzyloxy)-2-methoxyphenyl)-3-(4-fluorophenyl)-1H-pyrazole;
2-(3-(5-fluoro-2-methoxyphenyl)-1H-pyrazol-5-yl)pyridine;
3-(3,5-difluoro-2-methoxyphenyl)-5-(4-ethynylphenyl)-1H-pyrazole;
2-(3-(3-fluoro-2-methoxyphenyl)-1H-pyrazol-5-yl)pyridine;
3-(5-fluoro-2-methoxyphenyl)-5-(3-fluorophenyl)-1H-pyrazole;
3-(3-fluoro-2-methoxyphenyl)-5-(3-fluorophenyl)-1H-pyrazole;
5-(3-fluorophenyl)-3-(2-methoxyphenyl)-1H-pyrazole;
5-(4-fluorophenyl)-3-(1-methyl-1H-imidazol-2-yl)-1H-pyrazole;
3-benzyl-5-(2-methoxyphenyl)-4H-1,2,4-triazole;
3-benzyl-5-(4-tert-butylphenyl)-4H-1,2,4-triazole;
N-cyclopentyl-2-(5-methyl-4H-1,2,4-triazol-3-yl)aniline;
3-(2-(isopentyloxy)phenyl)-5-methyl-4H-1,2,4-triazole;
3-(2-(isopentyloxy)phenyl)-5-propyl-4H-1,2,4-triazole;
3-benzyl-5-(2-(isopentyloxy)phenyl)-4H-1,2,4-triazole;
3-benzyl-5-(2-isopropoxyphenyl)-4H-1,2,4-triazole;
5-(2-methoxyphenyl)-3-(trifluoromethyl)-1H-pyrazole;
5-(5-bromo-2-isobutoxyphenyl)-1H-pyrazole;
(E)-5-(2-isobutoxy-5-styrylphenyl)-1H-pyrazole;
5-(2-isobutoxy-5-phenethylphenyl)-1H-pyrazole;
5-(5-(benzyloxy)-2-(1-methoxypropan-2-yloxy)phenyl)-1H-pyrazol-3-ol;
5-(3-(2-fluorobenzyloxy)-5-(4-(methylsulfonyl)phenoxy)phenyl)-1H-pyrazol-3-ol;
(S)-5-(3-(2-fluorobenzyloxy)-5-(1-methoxypropan-2-yloxy)phenyl)-1H-pyrazol-3-ol;
(S)-5-(3-(1-methoxypropan-2-yloxy)-5-(4-(methylsulfonyl)phenoxy)phenyl)-1H-pyrazol-3-ol;
5-(3-(cyclopentyloxy)-5-(4-(methylsulfonyl)phenoxy)phenyl)-1H-pyrazol-3-ol;
5-(3-((3-methylpyridin-2-yl)methoxy)-5-(4-(methylsulfonyl)phenoxy)phenyl)-1H-pyrazol-3-ol;
5-(3-(2-methylbenzyloxy)-5-(4-(methylsulfonyl)phenoxy)phenyl)-1H-pyrazol-3-ol;
5-(5-(benzyloxy)-2-(1-methoxypropan-2-yloxy)phenyl)-3-methoxy-1H-pyrazole;
(S)-5-(3-(2-fluorobenzyloxy)-5-(1-methoxypropan-2-yloxy)phenyl)-3-methoxy-1H -pyrazole;
5-(3-(2-fluorobenzyloxy)-5-(4-(methylsulfonyl)phenoxy)phenyl)-3-methoxy-1H-pyrazole;
5-(3-(cyclopentyloxy)-5-(4-(methylsulfonyl)phenoxy)phenyl)-3-methoxy-1H-pyrazole;
2-((3-(3-methoxy-1H-pyrazol-5-yl)-5-(4-(methylsulfonyl)phenoxy)phenoxy)methyl)-3-methylpyridine;
3-methoxy-5-(3-(2-methylbenzyloxy)-5-(4-(methylsulfonyl)phenoxy)phenyl)-1H-pyrazole;
5-(2-(5-(5-(benzyloxy)-2-(1-methoxypropan-2-yloxy)phenyl)-1H-pyrazol-3-yloxy)ethyl)-4-methylthiazole;
(S)-5-(2-(5-(3-(2-fluorobenzyloxy)-5-(1-methoxypropan-2-yloxy)phenyl)-1H-pyrazol-3-yloxy)ethyl)-4-methylthiazole;
(S)-methyl 2-(5-(3-(1-methoxypropan-2-yloxy)-5-(4-(methylsulfonyl)phenoxy)phenyl)-1H-pyrazol-3-yloxy)acetate;
3-(3-methoxy-1H-pyrazol-5-yl)-4-(1-methoxypropan-2-yloxy)phenol;
3-(3-methoxy-1H-pyrazol-5-yl)-5-(4-(methylsulfonyl)phenoxy)phenol;
(S)-3-(3-methoxy-1H-pyrazol-5-yl)-5-(1-methoxypropan-2-yloxy)phenol;
5-(5-ethoxy-2-(1-methoxypropan-2-yloxy)phenyl)-3-methoxy-1H-pyrazole;
3-methoxy-5-(2-(1-methoxypropan-2-yloxy)-5-(2-(thiophen-3-yl)ethoxy)phenyl)-1H -pyrazole;
3-methoxy-5-(2-(1-methoxypropan-2-yloxy)-5-(4-(methylsulfonyl)benzyloxy)phenyl)-1H-pyrazole;
4-((3-(3-methoxy-1H-pyrazol-5-yl)-4-(1-methoxypropan-2-yloxy)phenoxy)methyl)pyridine;
(S)-3-methoxy-5-(3-(1-methoxypropan-2-yloxy)-5-(4-(methylsulfonyl)phenoxy)phenyl)-1H-pyrazole;
(S)-3-methoxy-5-(3-(1-methoxypropan-2-yloxy)-5-(4-(methylsulfonyl)benzyloxy)phenyl)-1H-pyrazole;
(S)-3-methoxy-5-(3-(1-methoxypropan-2-yloxy)-5-(2-(thiophen-3-yl)ethoxy)phenyl)-1H-pyrazole;
(S)-2-(5-(3-(1-methoxypropan-2-yloxy)-5-(4-(methylsulfonyl)phenoxy)phenyl)-1H -pyrazol-3-yloxy)acetic acid;
(S)-1-(4-acetylpiperazin-1-yl)-2-(5-(3-(1-methoxypropan-2-yloxy)-5-(4-(methylsulfonyl)phenoxy)phenyl)-1H-pyrazol-3-yloxy)ethanone;
(S)-2-(5-(3-(1-methoxypropan-2-yloxy)-5-(4-(methylsulfonyl)phenoxy)phenyl)-1H -pyrazol-3-yloxy)-1-morpholinoethanone;
(S)—N-ethyl-2-(5-(3-(1-methoxypropan-2-yloxy)-5-(4-(methylsulfonyl)phenoxy)phenyl)-1H-pyrazol-3-yloxy)acetamide;
1-isobutyl-6-(1H-pyrazol-5-yl)-1H-benzo [d]imidazole;
1-(cyclohexylmethyl)-6-(1H-pyrazol-5-yl)-1H-benzo[d]imidazole;
5-(1-isobutyl-1H-benzo[d]imidazol-6-yl)-1H-pyrazol-3-ol;
5-(1-(cyclohexylmethyl)-1H-benzo [d]imidazol-6-yl)-1H-pyrazol-3-ol;
1-isobutyl-6-(3-methoxy-1H-pyrazol-5-yl)-1H-benzo[d]imidazole;
1-(cyclohexylmethyl)-6-(3-methoxy-1H-pyrazol-5-yl)-1H-benzo[d]imidazole;
5-benzyl-3-phenyl-1H-1,2,4-triazole;
2-(3-phenyl-1H-1,2,4-triazol-5-yl)phenol;
(3-(2-ethoxyphenyl)-1H-1,2,4-triazol-5-yl)methanol;
1-(4-(methylthio)phenyl)-3-(5-phenyl-1H-1,2,4-triazol-3-yl)urea;
1-(5-phenyl-1H-1,2,4-triazol-3-yl)-3-(2-(trifluoromethyl)phenyl)urea;
1-(2,5-difluorophenyl)-3-(5-phenyl-1H-1,2,4-triazol-3-yl)urea;
5-(3,4,5-trimethoxyphenyl)-1H-1,2,4-triazol-3-amine;
N-(5-bromopyridin-2-yl)-2-(3-phenyl-1H-1,2,4-triazol-5-ylthio)acetamide;
2,2′-(1H-1,2,4-triazole-3,5-diyl)diphenol;
1-(3,4-dihydroxyphenyl)-2-(5-(2-methoxyphenyl)-1H-1,2,4-triazol-3-ylthio)ethanone;
2-(5-(2-chlorophenyl)-1H-1,2,4-triazol-3-ylthio)-N-cyclohexylacetamide;
(E)-2-(3-(4-bromostyryl)-1H-1,2,4-triazol-5-yl)phenol;
(E)-2-(3-(2-chlorostyryl)-1H-1,2,4-triazol-5-yl)phenol;
(E)-2-(3-(2-(3-methylthiophen-2-yl)vinyl)-1H-1,2,4-triazol-5-yl)phenol;
(E)-5-(2-(5-(2-hydroxyphenyl)-1H-1,2,4-triazol-3-yl)vinyl)-2-methoxyphenol;
3-(2-fluorobenzylthio)-5-(2-methoxyphenyl)-1H-1,2,4-triazole;
N-(3,4-dimethylphenyl)-2-(5-(2-methoxyphenyl)-1H-1,2,4-triazol-3-ylthio)acetamide;
N-(5-(3,4-dimethoxyphenyl)-1H-1,2,4-triazol-3-yl)-4,6-dimethylpyrimidin-2-amine;
2-(3-phenyl-1H-1,2,4-triazol-5-ylthio)-N-p-tolylacetamide;
3-(4-chlorophenyl)-1H-1,2,4-triazol-5-amine;
N-phenyl-2-(3-phenyl-1H-1,2,4-triazol-5-ylthio)acetamide;
N-(2-nitrophenyl)-2-(3-phenyl-1H-1,2,4-triazol-5-ylthio)acetamide;
N-(3,5-dibromopyridin-2-yl)-2-(3-phenyl-1H-1,2,4-triazol-5-ylthio)acetamide;
3-(2,4-dinitrophenyl)-1H-1,2,4-triazol-5-amine;
3-(2-fluorophenyl)-1H-1,2,4-triazol-5-amine;
N-(2,4-dimethylphenyl)-2-(3-(3-nitrophenyl)-1H-1,2,4-triazol-5-ylthio)acetamide;
3-(2-methoxyphenyl)-1H-1,2,4-triazol-5-amine;
3-(3,5-dimethoxyphenyl)-1H-1,2,4-triazol-5-amine;
5-(1H-pyrrol-2-yl)-3-(thiophen-2-yl)-1H-1,2,4-triazole;
N-(5-(2-methoxyphenyl)-1H-1,2,4-triazol-3-yl)-4,6-dimethylpyrimidin-2-amine;
3-(3-bromobenzylthio)-5-(2-methoxyphenyl)-1H-1,2,4-triazole;
2-(5-(2-methoxyphenyl)-1H-1,2,4-triazol-3-ylthio)-1-morpholinoethanone;
4-((5-(2-methoxyphenyl)-1H-1,2,4-triazol-3-ylthio)methyl)benzoic acid;
2-(5-(2-methoxyphenyl)-1H-1,2,4-triazol-3-ylthio)-N-(2-nitrophenyl)acetamide;
3-benzyl-5-(2-methoxyphenyl)-1H-1,2,4-triazole;
5-benzyl-3-(4-tert-butylphenyl)-1H-1,2,4-triazole;
N-cyclopentyl-2-(5-methyl-1H-1,2,4-triazol-3-yl)aniline;
2-(5-(2-isopropoxyphenyl)-1H-1,2,4-triazol-3-ylthio)acetic acid;
2-(5-(2-isopropoxyphenyl)-1H-1,2,4-triazol-3-ylthio)acetamide;
4-bromo-2-(1H-pyrazol-5-yl)phenol;
5-methyl-3-phenyl-1H-pyrazole-4-carboxamide;
4-chloro-2-(3-methyl-1H-pyrazol-5-yl)aniline;
3,5-diphenyl-1H-pyrazole;
2,4-difluoro-6-(1H-pyrazol-5-yl)phenol;
3-(4-chlorophenyl)-5-(3-phenyl-1H-pyrazol-4-yl)-1,2,4-oxadiazole;
4-ethyl-6-(4-(thiazol-4-yl)-1H-pyrazol-3-yl)benzene-1,3-diol;
4-methyl-6-(4-(2-methylthiazol-4-yl)-1H-pyrazol-3-yl)benzene-1,3-diol;
5-methoxy-3-methyl-2-(5-methyl-4-phenoxy-1H-pyrazol-3-yl)phenol;
4-(4-(4-methoxyphenyl)-5-methyl-1H-pyrazol-3-yl)benzene-1,3-diol;
3-(4-chlorophenyl)-5-(trifluoromethyl)-1H-pyrazole;
3-phenyl-1H-pyrazol-5-ol;
3,5-diphenyl-1H-pyrazol-4-ol;
5-(pyridin-2-yl)-1H-pyrazole-3-carboxylic acid;
2-(3-phenyl-1H-pyrazol-5-yl)pyridine;
N-(3-acetamidophenyl)-5-(pyridin-2-yl)-1H-pyrazole-3-carboxamide;
N-phenyl-5-(pyridin-2-yl)-1H-pyrazole-3-carboxamide;
N-(3-nitrophenyl)-5-(pyridin-2-yl)-1H-pyrazole-3-carboxamide;
N-(5-methylthiazol-2-yl)-5-(pyridin-2-yl)-1H-pyrazole-3-carboxamide;
5-(2,5-bis(2,2,2-trifluoroethoxy)phenyl)-1H-pyrazole;
3-(5-bromo-2-isobutoxyphenyl)-1H-pyrazole;
2-(5-methyl-1H-pyrazol-3-yl)-1H-benzo[d]imidazole;
2-(5-methyl-1H-pyrazol-3-yl)-1H-benzo[d]imidazol-4-ol;
2-(5-methyl-1H-pyrazol-3-yl)benzo[d]oxazol-4-amine;
7-bromo-2-(5-methyl-1H-pyrazol-3-yl)-3H-imidazo[4,5-c]pyridine;
2-(3-methyl-1H-pyrazol-5-yl)-7-phenyl-3H-imidazo[4,5-c]pyridine;
7-(3-(ethylsulfonyl)phenyl)-2-(5-methyl-1H-pyrazol-3-yl)-3H-imidazo[4,5-c]pyridine;
7-(1-(ethylsulfonyl)-1H-indol-6-yl)-2-(5-methyl-1H-pyrazol-3-yl)-3H-imidazo[4,5-c]pyridine;
N-(4-(2-(5-methyl-1H-pyrazol-3-yl)-3H-imidazo[4,5-c]pyridin-7-ylthio)phenyl)acetamide;
(E)-3-(2-isobutoxy-5-styrylphenyl)-1H-pyrazole;
5-(2,5-dimethoxyphenyl)-1H-pyrazole-3-carboxylic acid;
ethyl 5-phenyl-1H-pyrazole-3-carboxylate;
3-(2-isobutoxy-5-phenethylphenyl)-1H-pyrazole;
ethyl 5-(2-methoxyphenyl)-1H-pyrazole-3-carboxylate;
(5-(2-methoxyphenyl)-1H-pyrazol-3-yl)methanol;
2-(5-(2-methoxyphenyl)-1H-pyrazol-3-yloxy)acetic acid;
5-(3-fluorophenyl)-3-(1-methyl-1H-imidazol-2-yl)-1H-pyrazole;
3-(3,5-difluoro-2-methoxyphenyl)-5-(3-fluorophenyl)-1H-pyrazole;
2-(3-(3,5-difluoro-2-methoxyphenyl)-1H-pyrazol-5-yl)pyridine;
3,5-di(pyridin-2-yl)-1H-pyrazole;
ethyl 2-(5-(5-chloro-2-methoxyphenyl)-1H-1,2,4-triazol-3-ylthio)acetylcarbamate;
(R)-tert-butyl 3-((5-(2-methoxyphenyl)-1H-pyrazol-3-yloxy)methyl)-5-oxopiperazine-1-carboxylate;
3-(4-fluorophenyl)-5-(2-methoxyphenyl)-1H-pyrazole;
5-(2,3-dihydrobenzofuran-7-yl)-3-phenyl-1H-pyrazole;
5-(2,2-dimethyl-2,3-dihydrobenzofuran-7-yl)-3-phenyl-1H-pyrazole;
5-(5-bromo-2,3-dihydrobenzofuran-7-yl)-3-phenyl-1H-pyrazole;
4-(3-phenyl-1H-pyrazol-5-yl)benzo [c][1,2,5]thiadiazole;
2-(5-(2-isobutoxyphenyl)-1H-pyrazol-3-yl)pyridine;
3-(5-(2-isobutoxyphenyl)-1H-pyrazol-3-yl)pyridine;
2-(5-(5-chloro-2-isobutoxyphenyl)-1H-pyrazol-3-yl)pyridine;
3-(5-(5-chloro-2-isobutoxyphenyl)-1H-pyrazol-3-yl)pyridine;
5-(2-methoxy-5-nitrophenyl)-3-phenyl-1H-pyrazole;
3-(5-(2-methoxyphenyl)-1H-pyrazol-3-yl)aniline;
5-(4-(benzyloxy)-2-(2-methoxyethoxy)phenyl)-3-phenyl-1H-pyrazole;
3-(5-(2-methoxyphenyl)-1H-pyrazol-3-yl)pyridine;
4-(5-(5-(2-methoxyphenyl)-1H-pyrazol-3-yl)pyridin-2-yl)morpholine;
5-(1-benzyl-1H-imidazol-2-yl)-3-(3-fluorophenyl)-1H-pyrazole;
3-(5-(1-benzyl-1H-imidazol-2-yl)-1H-pyrazol-3-yl)pyridine;
2-(2-(3-(3-fluorophenyl)-1H-pyrazol-5-yl)phenoxy)-N,N-dimethylacetamide;
N,N-dimethyl-2-(2-(3-(pyridin-3-yl)-1H-pyrazol-5-yl)phenoxy)acetamide;
2-((5-(benzyloxy)-2-(3-phenyl-1H-pyrazol-5-yl)phenoxy)methyl)pyridine;
N-(3-(5-(2-methoxyphenyl)-1H-pyrazol-3-yl)phenyl)acetamide;
N-(3-(5-(2-methoxyphenyl)-1H-pyrazol-3-yl)phenyl)benzamide;
2-(2-(5-(4-cyanophenyl)-1H-pyrazol-3-yl)phenoxy)-N,N-dimethylacetamide;
2-(2-(5-(pyridin-3-yl)-1H-pyrazol-3-yl)phenoxy)-1-(pyrrolidin-1-yl)ethanone;
5-(5-(2-methoxyphenyl)-1H-pyrazol-3-yl)-N,N-dimethylpyridin-2-amine;
5-(3-(3-fluorophenyl)-1H-pyrazol-5-yl)-2,4-dimethoxypyrimidine;
2,4-dimethoxy-5-(3-(pyridin-3-yl)-1H-pyrazol-5-yl)pyrimidine;
3-(2-(3-phenyl-1H-pyrazol-5-yl)phenoxy)pyridine;
2-(3-(3-phenyl-1H-pyrazol-5-yl)phenoxy)pyridine;
5-(2-methoxy-4-nitrophenyl)-3-phenyl-1H-pyrazole;
4-(5-(2-methoxyphenyl)-1H-pyrazol-3-yl)aniline;
3-methoxy-4-(3-phenyl-1H-pyrazol-5-yl)aniline;
5-(2-methoxy-4-(4-methoxybenzyloxy)phenyl)-3-phenyl-1H-pyrazole;
N-(3-methoxy-4-(3-phenyl-1H-pyrazol-5-yl)phenyl)benzamide;
N-(3-methoxy-4-(3-phenyl-1H-pyrazol-5-yl)phenyl)-2-phenylacetamide;
2-(2-(3-(3-fluorophenyl)-1H-pyrazol-5-yl)phenoxy)-1-(pyrrolidin-1-yl)ethanone;
4-(5-(2-(2-oxo-2-(pyrrolidin-1-yl)ethoxy)phenyl)-1H-pyrazol-3-yl)benzonitrile;
2-(2-(3-(3-fluorophenyl)-1H-pyrazol-5-yl)phenoxy)-N,N-dimethylethanamine;
N-(2,4-dimethoxybenzyl)-5-(5-(2-methoxyphenyl)-1H-pyrazol-3-yl)pyridin-2-amine;
5-(5-(2-methoxyphenyl)-1H-pyrazol-3-yl)pyridin-2-amine;
3-(2-chlorophenyl)-5-(2-methoxyphenyl)-1H-pyrazole;
5-(2-methoxyphenyl)-3-o-tolyl-1H-pyrazole;
3-(2-chloro-6-fluorophenyl)-5-(2-methoxyphenyl)-1H-pyrazole;
N-(2-methoxyethyl)-5-(5-(2-methoxyphenyl)-1H-pyrazol-3-yl)pyridin-2-amine;
3-(5-(2-(3-chlorophenoxy)phenyl)-1H-pyrazol-3-yl)pyridine;
2-(2-(3-(pyridin-3-yl)-1H-pyrazol-5-yl)phenoxy)benzonitrile;
5-(6-methoxybenzofuran-5-yl)-3-phenyl-1H-pyrazole;
5-(2-methoxyphenyl)-1H-pyrazole-3-carboxylic acid;
5-(2-methoxyphenyl)-N-(3-methoxyphenyl)-1H-pyrazole-3-carboxamide;
1-((5-(2-methoxyphenyl)-1H-pyrazol-3-yl)methyl)-1H-benzo[d]imidazole;
3-((5-(2-methoxyphenyl)-1H-pyrazol-3-yl)methyl)quinazoline-2,4(1H,3H)-dione;
(5-(4-(benzyloxy)-2-methoxyphenyl)-1H-pyrazol-3-yl)methanamine;
6-methoxy-3-((5-(2-methoxyphenyl)-1H-pyrazol-3-yl)methyl)quinazolin-4(3H)-one;
6-((5-(4-(benzyloxy)-2-methoxyphenyl)-1H-pyrazol-3-yl)methylamino)-3-methylpyrimidine-2,4(1H,3H)-dione;
3-methoxy-N-((5-(2-methoxyphenyl)-1H-pyrazol-3-yl)methyl)aniline;
4-methoxy-3-(3-phenyl-1H-pyrazol-5-yl)aniline;
N-benzyl-4-methoxy-3-(3-phenyl-1H-pyrazol-5-yl)aniline;
N-benzyl-3-methoxy-4-(3-phenyl-1H-pyrazol-5-yl)aniline;
2-((5-(4-(benzyloxy)-2-methoxyphenyl)-1H-pyrazol-3-yl)methylamino)-6-fluoroquinazolin-4(3H)-one;
2-((5-(2-methoxyphenyl)-1H-pyrazol-3-yl)methyl)isoindoline-1,3-dione;
N-(4-methoxy-3-(3-phenyl-1H-pyrazol-5-yl)phenyl)benzamide;
N-(4-methoxy-3-(3-phenyl-1H-pyrazol-5-yl)phenyl)benzenesulfonamide;
N-(4-methoxy-3-(3-phenyl-1H-pyrazol-5-yl)phenyl)-2-phenylacetamide;
N-(4-methoxy-3-(3-phenyl-1H-pyrazol-5-yl)phenyl)isonicotinamide;
N-(4-methoxy-3-(3-phenyl-1H-pyrazol-5-yl)phenyl)thiazole-5-carboxamide;
N-(4-methoxy-3-(3-phenyl-1H-pyrazol-5-yl)phenyl)furan-3-carboxamide;
N-(4-methoxy-3-(3-phenyl-1H-pyrazol-5-yl)phenyl)tetrahydrofuran-3-carboxamide;
3-methoxy-5-(2-methoxyphenyl)-1H-pyrazole;
3-ethoxy-5-(2-methoxyphenyl)-1H-pyrazole;
5-(2-methoxyphenyl)-3-propoxy-1H-pyrazole;
3-isopropoxy-5-(2-methoxyphenyl)-1H-pyrazole;
3-sec-butoxy-5-(2-methoxyphenyl)-1H-pyrazole;
3-(cyclopentyloxy)-5-(2-methoxyphenyl)-1H-pyrazole;
5-(2-methoxyphenyl)-3-((tetrahydrofuran-2-yl)methoxy)-1H-pyrazole;
3-(2-methoxyethoxy)-5-(2-methoxyphenyl)-1H-pyrazole;
5-(2-methoxyphenyl)-3-(3-methoxypropoxy)-1H-pyrazole;
2-(5-(2-methoxyphenyl)-1H-pyrazol-3-yloxy)-N,N-dimethylethanamine;
3-(5-(2-methoxyphenyl)-1H-pyrazol-3-yloxy)-N,N-dimethylpropan-1-amine;
3-(benzyloxy)-5-(2-methoxyphenyl)-1H-pyrazole;
5-(2-methoxyphenyl)-3-phenethoxy-1H-pyrazole;
3-(3-fluorobenzyloxy)-5-(2-methoxyphenyl)-1H-pyrazole;
3-(4-fluorobenzyloxy)-5-(2-methoxyphenyl)-1H-pyrazole;
4-((5-(2-methoxyphenyl)-1H-pyrazol-3-yloxy)methyl)benzonitrile;
3-(2,4-difluorobenzyloxy)-5-(2-methoxyphenyl)-1H-pyrazole;
2-((5-(2-methoxyphenyl)-1H-pyrazol-3-yloxy)methyl)pyridine;
4-((5-(2-methoxyphenyl)-1H-pyrazol-3-yloxy)methyl)pyridine;
5-(2-methoxyphenyl)-3-(thiophen-2-ylmethoxy)-1H-pyrazole;
(S)-5-(2-methoxyphenyl)-3-(1-phenylethoxy)-1H-pyrazole;
N,N-diethyl-2-(5-(2-methoxyphenyl)-1H-pyrazol-3-yloxy)ethanamine;
5-(2-methoxyphenyl)-3-(2-(thiophen-3-yl)ethoxy)-1H-pyrazole;
1-(3-(5-(2-methoxyphenyl)-1H-pyrazol-3-yloxy)propyl)pyrrolidin-2-one;
1-(2-(5-(2-methoxyphenyl)-1H-pyrazol-3-yloxy)ethyl)imidazolidin-2-one;
2-(5-(2-methoxyphenyl)-1H-pyrazol-3-yloxy)-1-phenylethanone;
5-(2-(5-(2-methoxyphenyl)-1H-pyrazol-3-yloxy)ethyl)-4-methylthiazole;
2-((5-(2-methoxyphenyl)-1H-pyrazol-3-yloxy)methyl)thiazole;
5-(2-methoxyphenyl)-3-((1-methyl-1H-imidazol-5-yl)methoxy)-1H-pyrazole; and
5-(2-methoxyphenyl)-3-((1-methyl-1H-imidazol-2-yl)methoxy)-1H-pyrazole.

It is noted that the compounds of the present invention may be in the form of a pharmaceutically acceptable salt, biohydrolyzable ester, biohydrolyzable amide, biohydrolyzable carbamate, solvate, hydrate or prodrug thereof.

It is further noted that the compound may be present in a mixture of stereoisomers, or the compound may comprise a single stereoisomer. In addition, the compound may be present as a tautomer.

The present invention also provides a pharmaceutical composition comprising as an active ingredient a compound according to any one of the above embodiments and variations. In one particular variation, the composition is a solid formulation adapted for oral administration. In another particular variation, the composition is a liquid formulation adapted for oral administration. In yet another particular variation, the composition is a tablet. In still another particular variation, the composition is a liquid formulation adapted for parenteral administration.

In another of its aspects, there is provided a pharmaceutical composition comprising a compound according to any one of the above embodiments and variations, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In yet another of its aspects, there is provided a kit comprising a compound of any one of the above embodiments and variations; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another of its aspects, there is provided an article of manufacture comprising a compound of any one of the above embodiments and variations; and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the compound. In another variation, the article of manufacture comprises the compound in a multiple dose form.

In a further of its aspects, there is provided a therapeutic method comprising administering a compound of any one of the above embodiments and variations to a subject.

In another of its aspects, there is provided a method of activating glucokinase comprising contacting glucokinase with a compound of any one of the above embodiments and variations.

In yet another of its aspects, there is provided a method of activating glucokinase comprising causing a compound of any one of the above embodiments and variations to be present in a subject in order to activate glucokinase in vivo.

In a further of its aspects, there is provided a method of activating glucokinase comprising administering a first compound to a subject that is converted in vivo to a second compound wherein the second compound activates glucokinase in vivo, the second compound being a compound according to any one of the above embodiments and variations.

In another of its aspects, there is provided a method of treating a disease state for which increasing glucokinase activity ameliorates the pathology and/or symptomology of the disease state, the method comprising causing a compound of any one of the above embodiments and variations to be present in a subject in a therapeutically effective amount for the disease state.

In yet another of its aspects, there is provided a method of treating a disease state for which increasing glucokinase activity ameliorates the pathology and/or symptomology of the disease state, the method comprising administering a compound of any one of the above embodiments and variations to a subject, wherein the compound is present in the subject in a therapeutically effective amount for the disease state.

In a further of its aspects, there is provided a method of treating a disease state for which increasing glucokinase activity ameliorates the pathology and/or symptomology of the disease state, the method comprising administering a first compound to a subject that is converted in vivo to a second compound wherein the second compound activates glucokinase in vivo, the second compound being a compound according to any one of the above embodiments and variations.

In one variation of each of the above methods the disease state is selected from the group consisting of hyperglycemia, diabetes, dyslipidaemia, obesity, insulin resistance, metabolic syndrome X, impaired glucose tolerance, polycystic ovary syndrome and cardiovascular disease.

Salts, Hydrates, and Prodrugs of Glucokinase Activators

It should be recognized that the compounds of the present invention may be present and optionally administered in the form of salts, hydrates and prodrugs that are converted in vivo into the compounds of the present invention. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine(benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention that comprise basic nitrogen-containing groups may be quaternized with such agents as (C₁₋₄) alkyl halides, e.g., methyl, ethyl, iso-propyl and tert-butyl chlorides, bromides and iodides; di(C₁₋₄) alkyl sulfates, e.g., dimethyl, diethyl and diamyl sulfates; (C₁₀₋₁₈) alkyl halides, e.g., decyl, dodecyl, lauryl, myristyl and stearyl chlorides, bromides and iodides; and aryl(C₁₋₄) alkyl halides, e.g., benzyl chloride and phenethyl bromide. Such salts permit the preparation of both water-soluble and oil-soluble compounds of the present invention.

N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

Compounds of the present invention may also be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Preparation of Glucokinase Activators

Various methods may be developed for synthesizing compounds according to the present invention. Representative methods for synthesizing these compounds are provided in the Examples. It is noted, however, that compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) that they can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compositions Comprising Glucokinase Activators

A wide variety of compositions and administration methods may be used in conjunction with the compounds of the present invention. Such compositions may include, in addition to the compounds of the present invention, conventional pharmaceutical excipients, and other conventional, pharmaceutically inactive agents. Additionally, the compositions may include active agents in addition to the compounds of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The glucokinase activators and compositions comprising them may be administered or coadministered in any conventional dosage form. Co-administration in the context of this invention is intended to mean the administration of more than one therapeutic agent, one of which includes a glucokinase activator, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When compounds according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding compounds according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more compounds according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of an activator of the present invention to increase glucokinase activity in vivo, thereby treating the disease state of the subject.

Dosage forms or compositions may optionally comprise one or more compounds according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more glucokinase activators, optionally 0.1-95%, and optionally 1-95%.

Salts, preferably sodium salts, of the activators may be prepared with carriers that protect the compound against rapid elimination from the body, such as time release formulations or coatings. The formulations may further include other active compounds to obtain desired combinations of properties.

Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, compounds according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water-soluble FD and C dyes, mixtures thereof, and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The compounds of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising compounds of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

Injectables, Solutions, and Emulsions

The present invention is also directed to compositions designed to administer the compounds of the present invention by parenteral administration, generally characterized by subcutaneous, intramuscular or intravenous injection. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of an activator in the parenteral formulation may be adjusted so that an injection administers a pharmaceutically effective amount sufficient to produce the desired pharmacological effect. The exact concentration of an activator and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is know and practiced in the art.

Injectables may be designed for local and systemic administration. Typically a therapeutically effective dosage is formulated to contain a concentration of at least about 0.1% w/w up to about 90% w/w or more, preferably more than 1% w/w of the glucokinase activator to the treated tissue(s). The activator may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The glucokinase activator may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

Lyophilized Powders

The compounds of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a glucokinase activator is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the activator.

Topical Administration

The compounds of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The glucokinase activators may be formulated as aerosols for topical application, such as by inhalation (see, U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The activators may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the glucokinase activator alone or in combination with other pharmaceutically acceptable excipients can also be administered.

Formulations for Other Routes of Administrations

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

Examples of Formulations

The following are particular examples of oral, intravenous and tablet formulations that may optionally be used with compounds of the present invention. It is noted that these formulations may be varied depending on the particular compound being used and the indication for which the formulation is going to be used.

### Oral Formulation

| | | |
|---|---|---|
| Compound of the Present Invention | 10-100 | mg |
| Citric Acid Monohydrate | 105 | mg |
| Sodium Hydroxide | 18 | mg |
| Flavoring | | |
| Water | q.s. to 100 | mL |

### Intravenous Formulation

| Compound of the Present Invention | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

### Tablet Formulation

| | |
|---|---|
| Compound of the Present Invention | 1% |
| Microcrystalline Cellulose | 73% |
| Stearic Acid | 25% |
| Colloidal Silica | 1% |

Kits Comprising Glucokinase Activators

The invention is also directed to kits and other articles of manufacture for treating diseases associated with glucokinase. It is noted that diseases are intended to cover all conditions for which increasing glucokinase activity (e.g., upregulation of glucokinase) ameliorates the pathology and/or symptomology of the condition.

In one embodiment, a kit is provided that comprises a composition comprising at least one activator of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one activator of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Combination Therapy

A wide variety of therapeutic agents may have a therapeutic additive or synergistic effect with GK activators according to the present invention. In particular, the present invention also relates to the use of the GK activators of the present invention in combination with one or more other antidiabetic compounds. Examples of such other antidiabetic compounds include, but are not limited to S9 proteases, like dipeptidyl peptidase IV (DPP-IV) inhibitors; insulin signaling pathway modulators, like protein tyrosine phosphatase (PTPase) inhibitors, and glutamine-fructose-6-phosphate amidotransferase (GFAT) inhibitors; compounds influencing a dysregulated hepatic glucose production, like glucose-6-phosphatase (G6Pase) inhibitors, fructose-1,6-bisphosphatase (F-1,6-BPase) inhibitors, glycogen phosphorylase (GP) inhibitors, glucagon receptor antagonists and phosphoenolpyruvate carboxykinase (PEPCK) inhibitors; pyruvate dehydrogenase kinase (PDHK) inhibitors; insulin sensitivity enhancers (insulin sensitizers); insulin secretion enhancers (insulin secretagogues); alpha-glucosidase inhibitors; inhibitors of gastric emptying; other glucokinase (GK) activators; GLP-1 receptor agonists; UCP modulators; RXR modulators; GSK-3 inhibitors; PPAR modulators; metformin; insulin; and α₂-adrenergic antagonists. The compound of the present invention may be administered with such at least one other antidiabetic compound either simultaneously as a single dose, at the same time as separate doses, or sequentially (i.e., where one is administered before or after the other is administered).

In the case of combination therapy with compounds of the present invention, the other antidiabetic compound may be administered (e.g., route and dosage form) in a manner known per se for such compound. Compounds of the present invention and the other antidiabetic compound may be administered sequentially (i.e., at separate times) or at the same time, either one after the other separately in two separate dose forms or in one combined, single dose form. In one particular embodiment, the other antidiabetic compound is administered with compounds of the present invention as a single, combined dosage form. The dose of the antidiabetic compound may be selected from the range known to be clinically employed for such compound. Any of the therapeutic compounds of diabetic complications, antihyperlipemic compounds or antiobestic compounds can be used in combination with compounds of the present invention in the same manner as the above antidiabetic compounds.

### EXAMPLES

Preparation of Glucokinase Activators

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) and can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds according to the present invention may be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or thee-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| μL (microliters) | Ac (acetyl) |
| atm (atmosphere) | ATP (Adenosine Triphophatase) |
| BOC (tert-butyloxycarbonyl) | BOP (bis(2-oxo-3-oxa- |
| BSA (Bovine Serum Albumin) | zolidinyl)phosphinic chloride) |
| CDI (1,1-carbonyldiimidazole) | CBZ (benzyloxycarbonyl) |
| DCE (dichloroethane) | DCC (dicyclohexylcarbodiimide) |
| DMAP (4-dimethylaminopyridine) | DCM (dichloromethane) |
| DMF (N,N-dimethylformamide) | DME (1,2-dimethoxyethane) |
| DMSO (dimethylsulfoxide) | DMPU (N,N′-dimethyl- |
| EDTA (Ethylenediamine- | propyleneurea) |
| tetraacetic acid) | EDCI (ethylcarbodiimide |
| Et₂O (diethyl ether) | hydrochloride) |
| FMOC (9-fluorenyl- | Et (ethyl) |
| methoxycarbonyl) | EtOAc (ethyl acetate) |
| h (hours) | g (grams) |
| HOBT (1-hydroxybenzotriazole) | HOAc or AcOH (acetic acid) |
| HPLC (high pressure liquid | HOSu (N-hydroxysuccinimide) |
| chromatography) | Hz (Hertz) |
| i.v. (intravenous) | IBCF (isobutyl chloroformate) |
| i-PrOH (isopropanol) | L (liters) |
| M (molar) | mCPBA (meta-chloroperbenzoic |
| Me (methyl) | acid) |
| mg (milligrams) | MeOH (methanol) |
| min (minutes) | MHz (megahertz) |
| mM (millimolar) | mL (milliliters) |
| mol (moles) | mmol (millimoles) |
| mp (melting point) | MOPS (Morpholinepropanesulfonic |
| OMe (methoxy) | acid) |
| RP (reverse phase) | NaOAc (sodium acetate) |
| SPA (Scintillation Proximity | psi (pounds per square inch) |
| Assay) | RT (ambient temperature) |
| TBS (t-butyldimethylsilyl) | TBAF (tetra-n-butylammonium |
| TEA (triethylamine) | fluoride) |
| TFAA (trifluoroacetic anhydride) | tBu (tert-butyl) |
| TIPS (triisopropylsilyl) | TFA (trifluoroacetic acid) |
| TMS (trimethylsilyl) | THF (tetrahydrofuran) |
| Tr (retention time) | TLC (thin layer chromatography) |
| | TMSE (2-(trimethylsilyl)ethyl) |

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at RT unless otherwise noted.

¹H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad).

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60F-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosures of all documents cited throughout this application are incorporated herein by reference.

Synthetic Schemes for Compounds of the Present Invention

Compounds according to the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

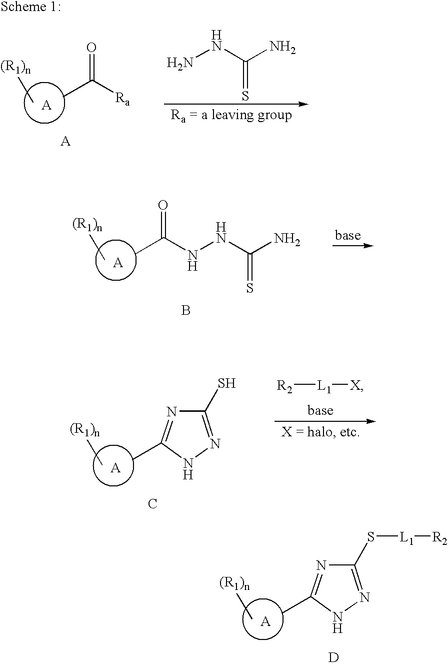

Referring to Scheme 1, Compound A (i.e., R′ is OH or halogen) is condensed with thiosemicarbazide to give Compound B. Compound B is treated with base (i.e., 1N NaOH) to give Compound C. S-alkylation of Compound C with R₂-L₁-X (i.e., X is halogen) yields Compound D.

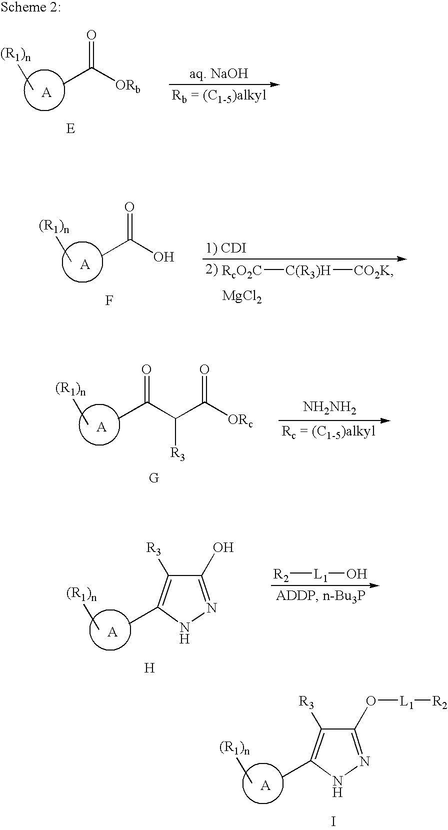

Referring to Scheme 2, Compound E is hydrolyzed with aqueous alkaline solution to give Compound F. Compound F is treated with CDI, and then treated with an alkyl malonate potassium salt and magnesium chloride to give Compound G. Compound G is treated with hydrazine to give Compound H. Mitsunobu reaction (i.e., azodicarbonyldipiperidine [ADDP] and n-Bu₃P) between Compound H and alcohol R₂-L₁-OH provides Compound I.

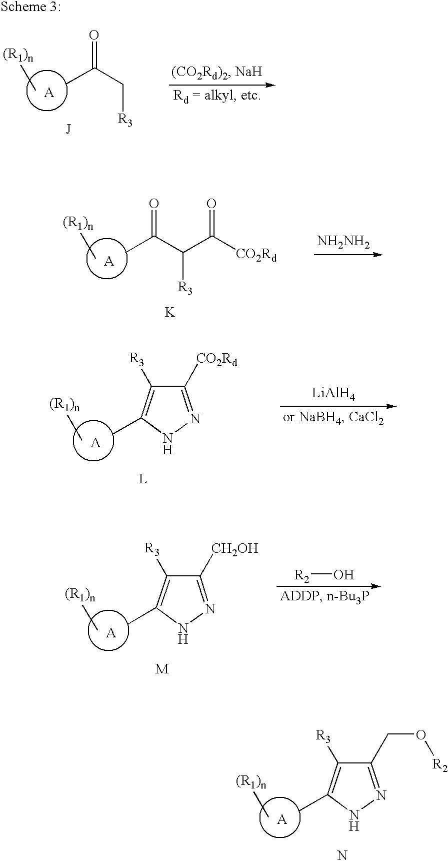

Referring to Scheme 3, Compound J is converted to Compound K with a dialkyl oxalate and NaH. Compound K is treated with hydrazine to give Compound L. Reduction of the ester group of Compound L provides Compound M. Mitsunobu reaction (i.e., ADDP and n-Bu₃P) between Compound M and alcohol R₂—OH provides Compound N.

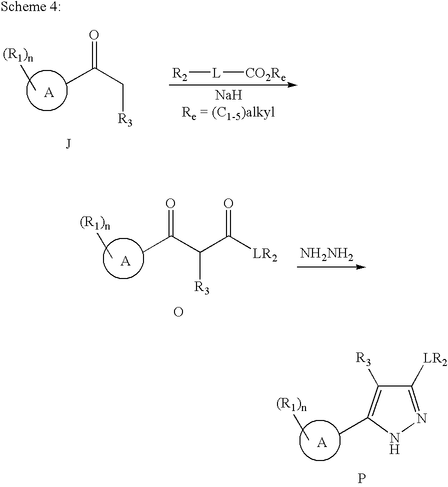

Referring to Scheme 4, Compound J is converted to Compound O with various esters (i.e., methyl benzoate) and NaH. Compound O is treated with hydrazine to give Compound P.

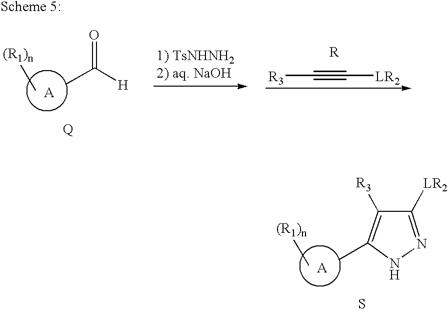

Referring to Scheme 5, Compound Q is condensed with tosylhydrazine, treated with aqueous NaOH, and then condensed with alkyne R to give Compound S.

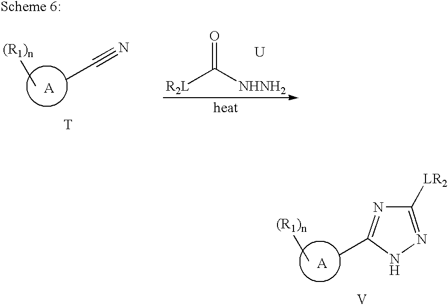

Referring to Scheme 6, Compound T is heated with Compound U to give Compound V.

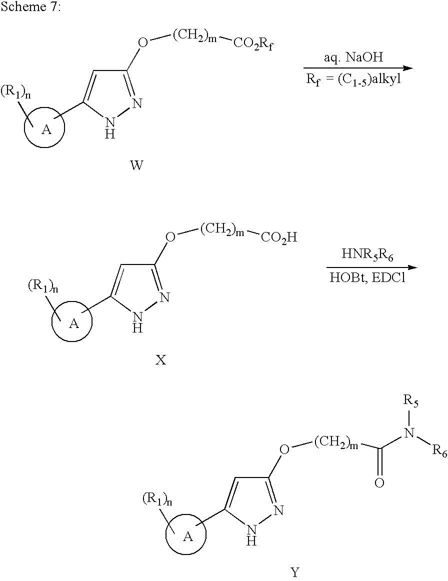

Referring to Scheme 7, Compound W is hydrolyzed with an aqueous alkaline solution to give Compound X. Compound X is condensed with an amine in the presence of condensation reagents (i.e., HOBt and EDCI) to give Compound Y.

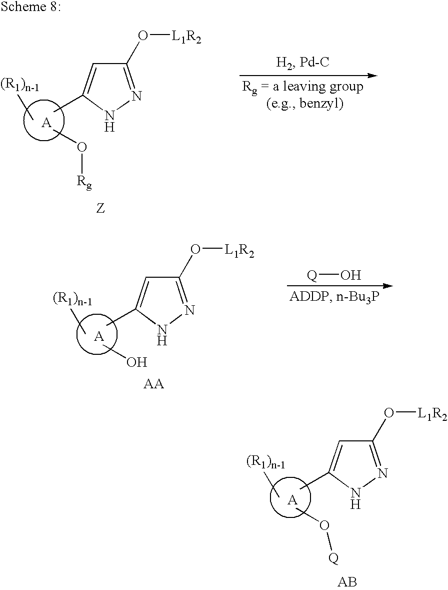

Referring to Scheme 8, Compound Z undergoes a catalytic hydrogenation reaction to give Compound AA. Mitsunobu reaction (i.e., ADDP and n-Bu₃P) between Compound AA and alcohol Q-OH provides Compound AB.

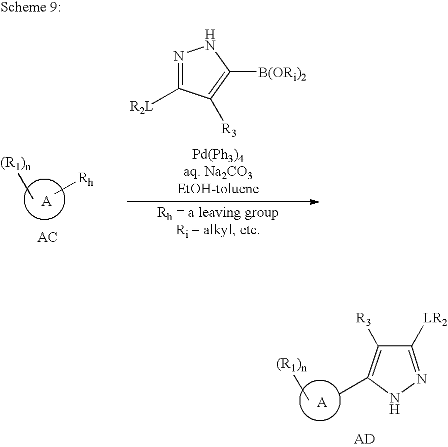

Referring to Scheme 9, Suzuki reaction (i.e., Pd(PPh₃)₄ and aq. Na₂CO₃) between Compound AC and a pyrazoleboronic acid (or borate) provides Compound AD.

Chiral components can be separated and purified using any of a variety of techniques known to those skilled in the art. For example, chiral components can be purified using supercritical fluid chromatography (SFC). In one particular variation, chiral analytical SFC/MS analyses are conducted using a Berger analytical SFC system (AutoChem, Newark, Del.) which consists of a Berger SFC dual pump fluid control module with a Berger FCM 1100/1200 supercritical fluid pump and FCM 1200 modifier fluid pump, a Berger TCM 2000 oven, and an Alcott 718 autosampler. The integrated system can be controlled by BI-SFC Chemstation software version 3.4. Detection can be accomplished with a Waters ZQ 2000 detector operated in positive mode with an ESI interface and a scan range from 200-800 Da with 0.5 second per scan. Chromatographic separations can be performed on a ChiralPak AD-H, ChiralPak AS-H, ChiralCel OD-H, or ChiralCel OJ-H column (5μ, 4.6×250 mm; Chiral Technologies, Inc. West Chester, Pa.) with 10 to 40% methanol as the modifier and with or without ammonium acetate (10 mM). Any of a variety of flow rates can be utilized including, for example, 1.5 or 3.5 mL/min with an inlet pressure set at 100 bar. Additionally, a variety of sample injection conditions can be used including, for example, sample injections of either 5 or 10 μL in methanol at 0.1 mg/mL in concentration.

In another variation, preparative chiral separations are performed using a Berger MultiGram II SFC purification system. For example, samples can be loaded onto a ChiralPak AD column (21×250 mm, 10μ). In particular variations, the flow rate for separation can be 70 mL/min, the injection volume up to 2 mL, and the inlet pressure set at 130 bar. Stacked injections can be applied to increase the efficiency.

In each of the above reaction procedures or schemes, the various substituents may be selected from among the various substituents otherwise taught herein.

Descriptions of the syntheses of particular compounds according to the present invention based on the above reaction scheme are set forth herein.

Examples of Glucokinase Activators

The present invention is further exemplified, but not limited by, the following examples that describe the synthesis of particular compounds according to the invention.

### Compound 3: 5-(2-methoxyphenyl)-3-(methylthio)-1H-1,2,4-triazole

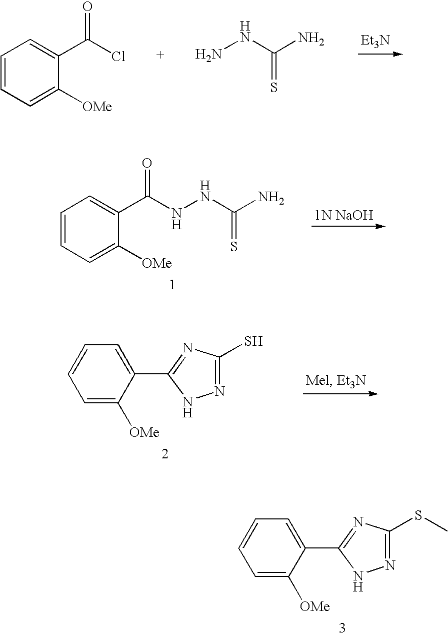

Referring to Scheme 1, o-anisoyl chloride (1.7 g) was reacted with thiosemicarbazide (0.9 g) in dichloromethane (20 ml) and pyridine (1.2 ml) for 15 hours at room temperature. The reaction mixture was diluted with water and the precipitate was collected by filtration to give Compound 1. Compound 1 was suspended in 1N NaOH (20 ml) and heated at 70° C. for 3 hours. The reaction mixture was cooled to room temperature and acidified with 1N HCl. The precipitate was collected by filtration to give Compound 2. Compound 2 (100 mg) and triethylamine (49 mg) were dissolved in DMF and iodomethane (0.03 ml) was added. After stirring at room temperature for 2 h, the reaction mixture was concentrated in vacuo. Purification by HPLC afforded the title Compound 3 as a pale yellow oily solid (12 mg). ¹H NMR (400 MHz, CHLOROFORM-d) δ 2.57-2.69 (m, 2H) 4.02 (s, 3H) 7.03-7.14 (m, 1H) 7.21 (d, J=8.08 Hz, 1H) 7.44-7.57 (m, 1H) 8.02 (dd, J=7.83, 1.52 Hz, 1H). [M+H] calc'd for C₁₀H₁₁N₃OS, 222; found, 222.

### Compound 4: 5-(2-ethoxyphenyl)-3-(2-fluorobenzylthio)-1H-1,2,4-triazole

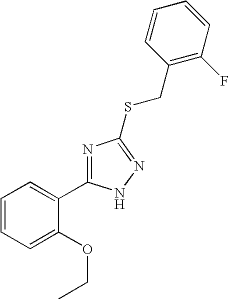

The title compound was synthesized using an analogous procedure to that described in connection with Compound 3 except that 2-ethoxybenzoic acid and 2-fluorobenzyl chloride were used. ¹H NMR (400 MHz, METHANOL-d₄) δ 1.46 (t, J=6.95 Hz, 3H) 4.30 (q, J=6.99 Hz, 2H) 4.41 (s, 2H) 7.01-7.11 (m, 3H) 7.17 (d, J=8.34 Hz, 1H) 7.20-7.32 (m, 1H) 7.37-7.52 (m, 2H) 7.96 (dd, J=7.71, 1.64 Hz, 2H). [M+H] calc'd for C₁₇H₁₆FN₃OS 330; found, 330.

### Compound 5: 5-(2-(benzyloxy)phenyl)-3-(2-fluorobenzylthio)-1H-1,2,4-triazole

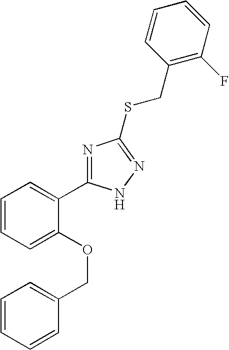

The title compound was synthesized using an analogous procedure to that described in connection with Compound 3 except that 2-benzyloxybenzoic acid and 2-fluorobenzyl chloride were used. ¹H NMR (400 MHz, METHANOL-d₄) δ 4.41 (s, 2H) 5.27-5.44 (m, 2H) 6.96-7.61 (m, 12H) 7.91-8.06 (m, 1H). [M+H] calc'd for C₂₂H₁₈FN₃OS 392; found, 392.

### Compound 6: 5-(2,4-dimethoxyphenyl)-3-(2-fluorobenzylthio)-1H-1,2,4-triazole

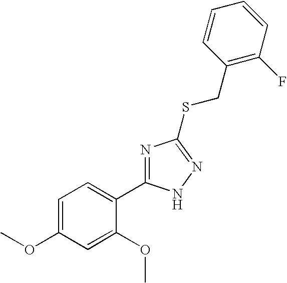

The title compound was synthesized using an analogous procedure to that described in connection with Compound 3 except that 2,4-dimethoxybenzoic acid and 2-fluorobenzyl chloride were used. [M+H] calc'd for C₁₇H₁₆FN₃O₂S 346; found, 346.

### Compound 7: 5-(2,5-dimethoxyphenyl)-3-(2-fluorobenzylthio)-1H-1,2,4-triazole

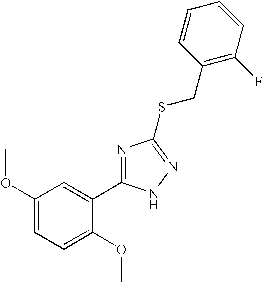

The title compound was synthesized using an analogous procedure to that described in connection with Compound 3 except that 2,5-dimethoxybenzoic acid and 2-fluorobenzyl chloride were used. [M+H] calc'd for C₁₇H₁₆FN₃O₂S 346; found, 346.

### Compound 8: 2-((5-(2-methoxyphenyl)-1H-1,2,4-triazol-3-ylthio)methyl)pyridine

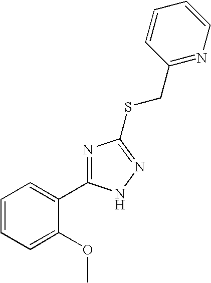

The title compound was synthesized using an analogous procedure to that described in connection with Compound 3 except that 2-pyridylmethyl chloride hydrochloride was used. ¹H NMR (400 MHz, DMSO-d₆) δ 3.92 (s, 3H) 4.51 (s, 2H) 7.07 (t, J=7.58 Hz, 1H) 7.18 (d, J=8.34 Hz, 1H) 7.30-7.39 (m, 1H) 7.42-7.52 (m, 1H) 7.58 (d, J=7.83 Hz, 1H) 7.79-7.89 (m, 1H) 7.96-8.06 (m, 1H) 8.55 (d, J=4.29 Hz, 1H). [M+H] calc'd for C₁₅H₁₄N₄OS 299; found, 299.

### Compound 9: 3-((5-(2-methoxyphenyl)-1H-1,2,4-triazol-3-ylthio)methyl)pyridine

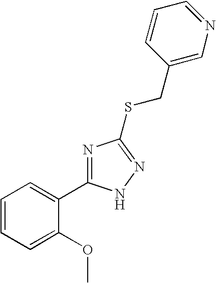

The title compound was synthesized using an analogous procedure to that described in connection with Compound 3 except that 3-pyridylmethyl chloride hydrochloride was used. ¹H NMR (400 MHz, CHLOROFORM-d) δ 3.92 (s, 3H) 4.44 (s, 2H) 7.08 (t, J=7.45 Hz, 1H) 7.18 (d, J=8.34 Hz, 1H) 7.41-7.57 (m, 2H) 7.98-8.12 (m, 1H) 8.43-8.55 (m, 1H) 8.72 (d, J=1.01 Hz, 1H). [M+H] calc'd for C₁₅H₁₄N₄OS 299; found, 299.

### Compound 10: 2-(5-(2-methoxyphenyl)-1H-1,2,4-triazol-3-ylthio)-N,N-dimethylacetamide

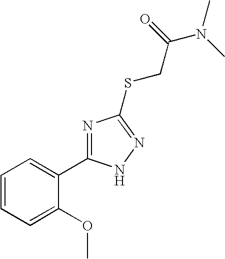

The title compound was synthesized using an analogous procedure to that described in connection with Compound 3 except that 2-chloro-N,N-dimethylacetamide was used. ¹H NMR (400 MHz, DMSO-d₆) δ 2.86 (s, 3H) 3.08 (s, 3H) 3.95 (s, 3H) 4.20 (s, 2H) 7.09 (t, J=7.58 Hz, 1H) 7.20 (d, J=8.59 Hz, 1H) 7.41-7.54 (m, 1H) 8.02 (d, J=7.58 Hz, 1H) 13.71 (s, 1H). [M+H] calc'd for C₁₃H₁₆N₄O₂S 293; found, 293.

### Compound 12: 2-(5-(2-isopropoxyphenyl)-1H-1,2,4-triazol-3-ylthio)-1-morpholinoethanone

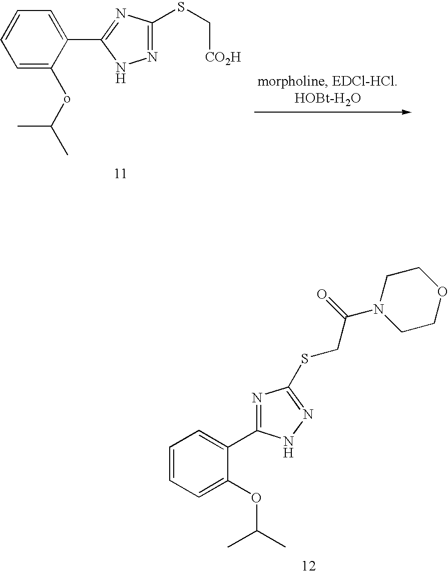

Compound 11 (147 mg) was reacted with morpholine (0.04 ml), EDCI-HCl (144 mg), and HOBt-hydrate (115 mg) in dichloromethane (5 ml) for 15 hours at room temperature. The reaction mixture was diluted with water and extracted with dichloromethane. The extract was washed with brine, dried over Na₂SO₄, and concentrated in vacuo. Purification by HPLC afforded the title Compound 12 as a colorless solid (30 mg). ¹H NMR (400 MHz, DMSO-d₆) δ 0.90 (m, 4H) 1.71-1.84 (m, 2H) 3.37-3.64 (m, 8H) 4.14 (t, J=6.69 Hz, 1H) 4.19 (s, 2H) 6.99-7.08 (m, 1H) 7.19 (d, J=8.34 Hz, 1H) 7.36-7.48 (m, 1H) 7.90 (dd, J=7.83, 1.52 Hz, 1H). [M+H] calc'd for C₁₇H₂₂N₄O₃S, 363; found, 363.

### Compound 13: 2-(5-(2-isopropoxyphenyl)-1H-1,2,4-triazol-3-ylthio)-N,N-dimethylacetamide

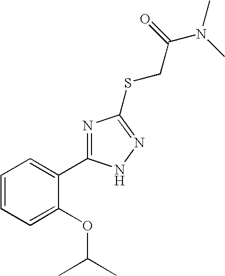 The title compound was synthesized using an analogous procedure to that described in connection with Compound 12 except that N,N-dimethylamine was used. [M+H] calc'd for C₁₅H₂₀N₄O₂S, 320; found, 320.

### Compound 16: 3-(2-fluorobenzyloxy)-5-(2-methoxyphenyl)-1H-pyrazole

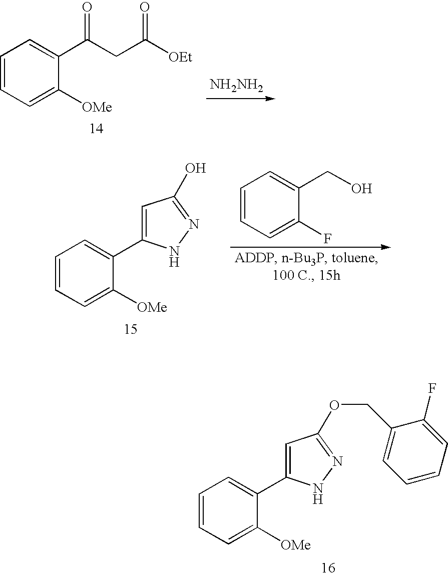

Referring to Scheme 2, Compound 14 (4.4 g) was reacted with hydrazine monohydrate (1.0 ml) in ethanol (20 ml) for 15 hours at room temperature. The reaction mixture was diluted with water and the precipitate was collected by filtration to give Compound 15. A solution of Compound 15 (285 mg), 2-fluorobenzyl alcohol (189 mg), ADDP (378 mg) and tri-n-butylphosphine (303 mg) was heated in toluene (5 ml) at 100° C. for 15 hours. The reaction mixture was diluted with ethyl acetate and the precipitate was removed by filtration. The filtrate was washed with aqueous NaHCO₃ solution and brine, dried over Na₂SO₄, and concentrated in vacuo. Purification by HPLC afforded the title Compound 16 as a pale pink solid (80 mg). ¹H NMR (400 MHz, DMSO-d₆) δ 3.87 (s, 3H) 5.24 (s, 2H) 6.19 (s, 1H) 7.01 (t, J=7.45 Hz, 1H) 7.12 (d, J=8.08 Hz, 1H) 7.19-7.28 (m, 2H) 7.29-7.37 (m, 1H) 7.37-7.45 (m, 1H) 7.53-7.60 (m, 1H) 7.64 (dd, J=7.58, 1.52 Hz, 1H) 12.07 (s, 1H). [M+H] calc'd for C₁₇H₁₅FN₂O₂, 299; found, 299.

### Compound 17: 3-(2-fluorobenzyloxy)-5-phenyl-1H-pyrazole

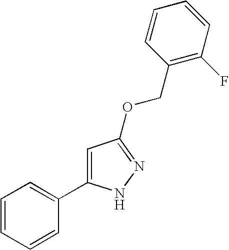

The title compound was synthesized using an analogous procedure to that described in connection with Compound 16 except that ethyl benzoylacetate was used. ¹H NMR (400 MHz, CHLOROFORM-d) δ 5.34 (s, 2H) 6.04 (s, 1H) 7.09 (t, J=9.22 Hz, 1H) 7.17 (t, J=7.45 Hz, 1H) 7.28-7.47 (m, 4H) 7.49-7.59 (m, 3H). [M+H] calc'd for C₁₆H₁₃FN₂O 269; found, 269.

### Compound 18: 3-(2-fluorobenzyloxy)-5-(2-fluorophenyl)-1H-pyrazole

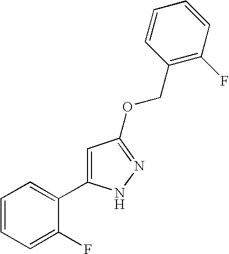

The title compound was synthesized using an analogous procedure to that described in connection with Compound 16 except that ethyl 2-fluorobenzoylacetate was used. ¹H NMR (400 MHz, CHLOROFORM-d) δ 5.35 (s, 2H) 6.14 (s, 1H) 7.05-7.13 (m, 1H) 7.13-7.25 (m, 3H) 7.28-7.38 (m, 2H) 7.51-7.63 (m, 2H). [M+H] calc'd for C₁₆H₁₂F₂N₂O 287; found, 287.

### Compound 19: 5-(3,5-dimethoxyphenyl)-3-(2-fluorobenzyloxy)-1H-pyrazole

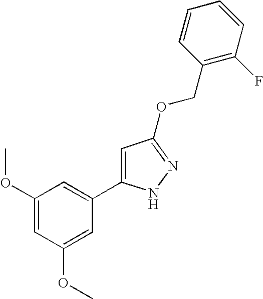

The title compound was synthesized using an analogous procedure to that described in connection with Compound 16 except that ethyl 3,5-dimethoxybenzoylacetate was used. ¹H NMR (400 MHz, DMSO-d₆) δ 3.73-3.88 (m, 6H) 5.23 (s, 2H) 6.27 (s, 1H) 6.48 (t, J=2.15 Hz, 1H) 6.88 (d, J=2.27 Hz, 2H) 7.15-7.32 (m, 2H) 7.35-7.47 (m, 1H) 7.47-7.66 (m, 1H) 12.11-12.75 (m, 1H). [M+H] calc'd for C₁₈H₁₇FN₂O₃ 329; found, 329.

### Compound 20: 3-(2-fluorobenzyloxy)-5-o-tolyl-1H-pyrazole

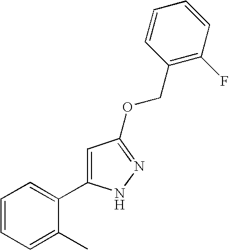

The title compound was synthesized using an analogous procedure to that described in connection with Compound 16 except that ethyl 2-methylbenzoylacetate was used. ¹H NMR (400 MHz, DMSO-d₆) δ 2.36 (s, 3H) 5.24 (s, 2H) 5.95 (s, 1H) 7.17-7.34 (m, 5H) 7.36-7.47 (m, 2H) 7.52-7.66 (m, 1H) 11.77-12.53 (m, 1H). [M+H] calc'd for C₁₇H₁₅FN₂O 283; found, 283.

### Compound 21: 5-(2,4-dimethoxyphenyl)-3-(2-fluorobenzyloxy)-1H-pyrazole

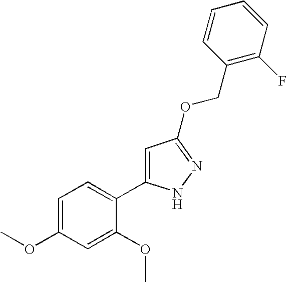

The title compound was synthesized using an analogous procedure to that described in connection with Compound 16 except that ethyl 2,4-dimethoxybenzoylacetate was used. [M+H] calc'd for C₁₈H₁₇FN₂O₃ 329; found, 329.

### Compound 25: 3-((2-fluorophenoxy)methyl)-5-(2-methoxyphenyl)-1H-pyrazole

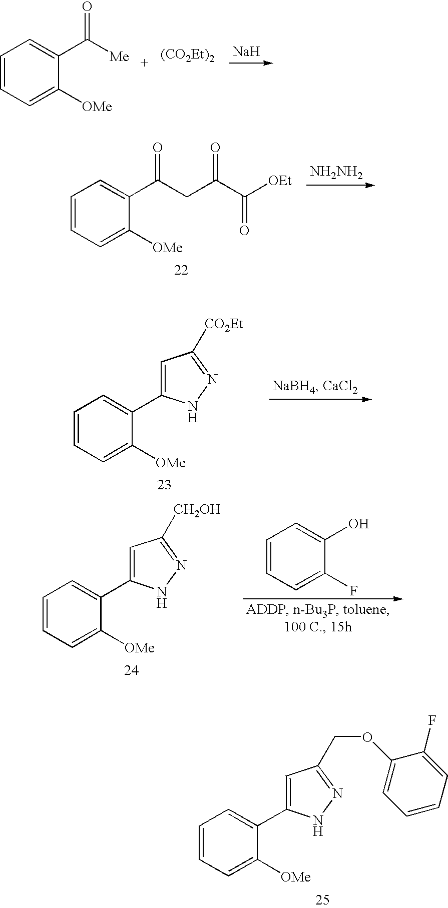

Referring to Scheme 3,2-methoxyacetophenone (4.5 g) was reacted with diethyl oxalate (8.2 ml) and sodium hydride (60% in oil; 3.6 g) in toluene (40 ml) at 80° C. for 15 hours. The reaction mixture was cooled to room temperature and then quenched with water. The mixture was acidified with 1N HCl and extracted with ethyl acetate. The extract was washed with brine, dried over Na₂SO₄, and concentrated in vacuo to give Compound 22. A solution of Compound 22 and hydrazine monohydrate (0.5 ml) in ethanol (10 ml) was stirred for 15 hours at room temperature. The reaction mixture was diluted with water extracted with ethyl acetate. The extract was washed with brine, dried over Na₂SO₄, concentrated in vacuo, and purified by silica gel chromatography to give Compound 23. To a suspension of calcium chloride (444 mg) and sodium borohydride (303 mg) in ethanol (5 ml) and THF (2 ml) was added a solution of Compound 23 (246 mg) in THF (3 ml) at 0° C. and the resulting mixture was stirred at 0° C. for 15 hours. The reaction mixture was acidified with 1N HCl and concentrated in vacuo. The residue was diluted with water and extracted with ethyl acetate. The extract was washed with brine, dried over Na₂SO₄, and concentrated in vacuo to give Compound 24. A solution of Compound 24 (204 mg), 2-fluorophenol (112 mg), ADDP (252 mg) and tri-n-butylphosphine (202 mg) was heated in toluene (5 ml) at 100° C. for 15 hours. The reaction mixture was diluted with ethyl acetate and the precipitate was removed by filtration. The filtrate was washed with aqueous NaHCO₃ solution and brine, dried over Na₂SO₄, and concentrated in vacuo. Purification by HPLC afforded the title Compound 25 as a pale pink solid (15 mg). ¹H NMR (400 MHz, DMSO-d₆) δ 3.79-3.92 (m, 3H) 5.09-5.17 (m, 2H) 6.72-7.41 (m, 7H) 7.62-7.94 (m, 1H) 12.83-13.05 (m, 1H). [M+H] calc'd for C₁₇H₁₅FN₂O₂, 299; found, 299.

### Compound 27: 5-(2-methoxyphenyl)-3-phenyl-1H-pyrazole

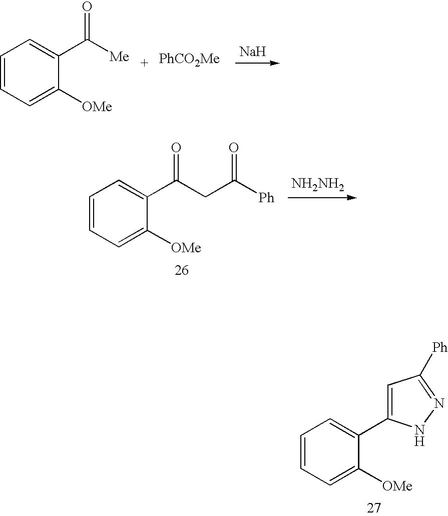

Referring to Scheme 3,2-methoxyacetophenone (1.5 g) was reacted with methyl benzoate (2.5 ml) and sodium hydride (60% in oil; 1.6 g) in toluene (30 ml) at 80° C. for 15 hours. The reaction mixture was cooled to room temperature and then quenched with water. The mixture was acidified with 1N HCl and extracted with ethyl acetate. The extract was washed with brine, dried over Na₂SO₄, and concentrated in vacuo to give Compound 26. A solution of Compound 26 and hydrazine monohydrate (0.7 ml) in ethanol (20 ml) was stirred for 15 hours at room temperature. The reaction mixture was diluted with water and extracted with ethyl acetate. The extract was washed with brine, dried over Na₂SO₄, concentrated in vacuo, and purified by silica gel chromatography to give the title Compound 27 as a pale yellow oil (1.2 g). ¹H NMR (400 MHz, DMSO-d₆) δ 3.93 (s, 3H) 6.75-8.05 (m, 10H) 12.66-13.44 (m, 1H). [M+H] calc'd for C₁₆H₁₄N₂O, 251; found, 251.

### Compound 28: 3-cyclopropyl-5-(2-methoxyphenyl)-1H-pyrazole

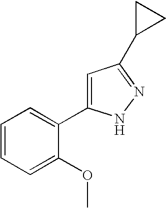

The title compound was synthesized using an analogous procedure to that described in connection with Compound 27 except that methyl cyclopropylcarboxylate was used. ¹H NMR (400 MHz, DMSO-d₆) δ 0.67-0.79 (m, 2H) 0.85-0.97 (m, 2H) 1.86-2.02 (m, 1H) 3.87 (s, 3H) 6.47 (s, 1H) 6.93-7.04 (m, 1H) 7.11 (d, J=8.34 Hz, 1H) 7.24-7.36 (m, 1H) 7.72 (dd, J=7.71, 1.64 Hz, 1H). [M+H] calc'd for C₁₃H₁₄N₂O 215; found, 215.

### Compound 29: 3-(4-chlorophenyl)-5-(2-methoxyphenyl)-1H-pyrazole

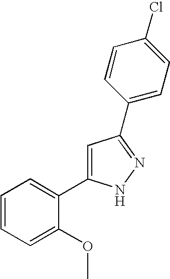

The title compound was synthesized using an analogous procedure to that described in connection with Compound 27 except that methyl 2-methoxybenzoate and 4-chloroacetophenone was used. ¹H NMR (400 MHz, CHLOROFORM-d) δ 4.02 (s, 3H) 6.91 (s, 1H) 7.00-7.14 (m, 2H) 7.31-7.45 (m, 3H) 7.73 (d, J=7.33 Hz, 1H) 7.81 (d, J=8.34 Hz, 2H) 11.33 (s, 1H). [M+H] calc'd for C₁₆H₁₃ClN₂O 285; found, 285.

### Compound 31: 2-(3-phenyl-1H-pyrazol-5-yl)phenol

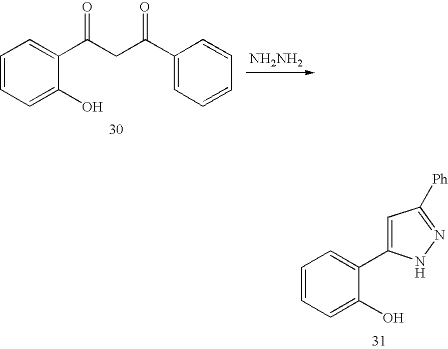

Referring to Scheme 4, Compound 30 (1.2 g) was reacted with hydrazine monohydrate (1.0 ml) in ethanol (20 ml) for 15 hours at room temperature. The reaction mixture was diluted with water and the precipitate was collected by filtration to give Compound 31 as a beige solid (1.1 g). ¹H NMR (400 MHz, CHLOROFORM-d) δ 6.91-6.99 (m, 2H) 7.02-7.09 (m, 1H) 7.20-7.29 (m, 2H) 7.40-7.55 (m, 3H) 7.57-7.68 (m, 2H) 10.11 (s, 1H) 10.74 (s, 1H). [M+H] calc'd for C₁₅H₁₂N₂O, 237; found, 237.

### Compound 32: 4-bromo-2-(3-phenyl-1H-pyrazol-5-yl)phenol

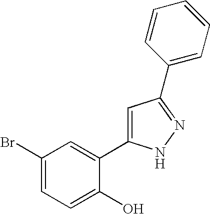

The title compound was synthesized using an analogous procedure to that described in connection with Compound 31 except that 1-(5-bromo-2-hydroxyphenyl)-3-phenylpropane-1,3-dione was used. ¹H NMR (400 MHz, CHLOROFORM-d) δ 6.90 (s, 1H) 6.93 (d, J=8.59 Hz, 1H) 7.32 (dd, J=8.72, 2.40 Hz, 1H) 7.40-7.55 (m, 3H) 7.58-7.65 (m, 2H) 7.74 (d, J=2.53 Hz, 1H) 10.16 (s, 1H) 10.80 (s, 1H). [M+H] calc'd for C₁₅H₁₁BrN₂O 316; found, 316.

### Compound 35: 5-(2,3-dimethoxyphenyl)-3-(2-fluorobenzyloxy)-1H-pyrazole

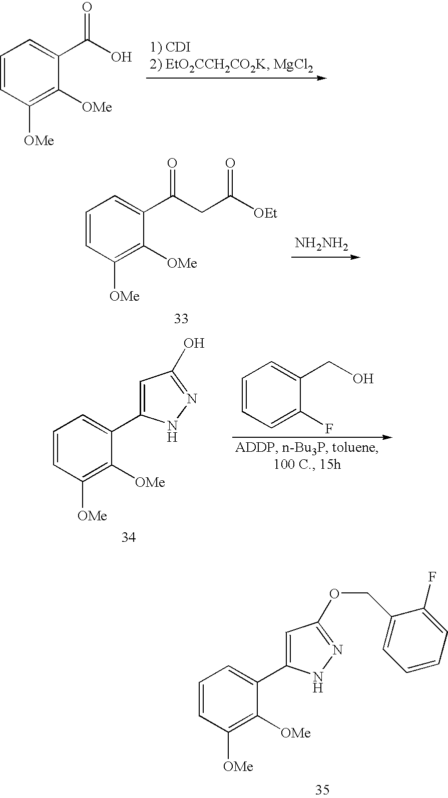

Referring to Scheme 2,2,3-dimethoxybenzoic acid (1.8 g) was reacted with N,N-carbonyldiimidazole (2.0 g) in THF (5 ml) at room temperature for 5 hours. A suspension of magnesium dichloride (1.3 g) and ethyl malonate potassium salt (1.9 g) in THF (20 ml) was heated at 50° C. for 5 hours. After cooling to room temperature, the first solution was added to the suspension. The resulting mixture was stirred at room temperature for 15 hours, diluted with water, and extracted with ethyl acetate. The extract was washed with aqueous NaHSO₄ solution and brine, dried over Na₂SO₄, and concentrated in vacuo. Purification by silica gel chromatography afforded Compound 33. Compound 33 was reacted with hydrazine monohydrate (0.2 ml) in ethanol (2 ml) for 15 hours at room temperature. The reaction mixture was diluted with water and the precipitate was collected by filtration to give Compound 34. A solution of Compound 34 (220 mg), 2-fluorobenzyl alcohol (126 mg), ADDP (252 mg) and tri-n-butylphosphine (202 mg) was heated in toluene (5 ml) at 100° C. for 15 hours. The reaction mixture was diluted with ethyl acetate and the precipitate was removed by filtration. The filtrate was washed with aqueous NaHCO₃ solution and brine, dried over Na₂SO₄, and concentrated in vacuo. Purification by silica gel chromatography afforded the title Compound 35 as a pale beige solid (50 mg). ¹H NMR (400 MHz, DMSO-d₆) δ 3.71 (s, 3H) 3.84 (s, 3H) 5.24 (s, 2H) 6.17 (s, 1H) 7.02-7.07 (m, 1H) 7.12 (t, J=7.96 Hz, 1H) 7.20-7.29 (m, 3H) 7.37-7.46 (m, 1H) 7.52-7.62 (m, 1H) 12.16 (s, 1H). [M+H] calc'd for C₁₈H₁₇FN₂O₃ 329; found, 329.

### Compound 36: 5-(2,3-dihydrobenzofuran-7-yl)-3-(2-fluorobenzyloxy)-1H-pyrazole

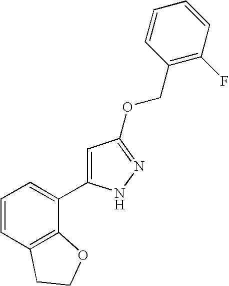

The title compound was synthesized using an analogous procedure to that described in connection with Compound 35 except that 2,3-dihydrobenzofuran-7-carboxylic acid was used. ¹H NMR (400 MHz, DMSO-d₆) δ 3.23 (t, J=8.72 Hz, 2H) 4.64 (t, J=8.84 Hz, 2H) 5.23 (s, 2H) 6.16 (s, 1H) 6.90 (t, J=7.58 Hz, 1H) 7.18-7.28 (m, 3H) 7.36-7.44 (m, 1H) 7.47 (d, J=7.33 Hz, 1H) 7.52-7.62 (m, 1H) 11.99-12.37 (m, 1H). [M+H] calc'd for C₁₈H₁₅FN₂O₂ 311; found, 311.

### Compound 37: 5-(5-(benzyloxy)-2-isopropoxyphenyl)-3-(2-fluorobenzyloxy)-1H-pyrazole

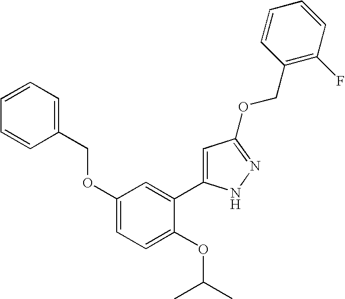

The title compound was synthesized using an analogous procedure to that described in connection with Compound 35 except that 5-benzyloxy-2-isopropoxybenzoic acid was used. ¹H NMR (400 MHz, METHANOL-d₄) δ 1.29-1.35 (m, 6H) 4.51-4.62 (m, 1H) 5.08 (s, 2H) 5.26 (s, 2H) 6.11-6.36 (m, 1H) 6.89-7.61 (m, 13H). [M+H] calc'd for C₂₆H₂₅FN₂O₃ 433; found, 433.

### Compound 38: 5-(2,5-dimethoxyphenyl)-3-(2-fluorobenzyloxy)-1H-pyrazole

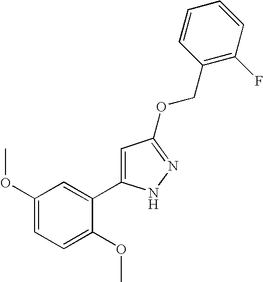

The title compound was synthesized using an analogous procedure to that described in connection with Compound 35 except that 2,5-dimethoxybenzoic acid was used. ¹H NMR (400 MHz, DMSO-d₆) δ 3.75 (s, 3H) 3.82 (s, 3H) 5.24 (s, 2H) 6.25 (s, 1H) 6.91 (dd, J=8.84, 2.02 Hz, 1H) 7.04 (d, J=9.09 Hz, 1H) 7.16-7.30 (m, 3H) 7.41 (q, J=6.65 Hz, 1H) 7.57 (t, J=7.45 Hz, 1H) 12.09 (s, 1H). [M+H] calc'd for C₁₈H₁₇FN₂O₃ 329; found, 329.

### Compound 39: 5-(5-fluoro-2-methoxyphenyl)-3-(2-fluorobenzyloxy)-1H-pyrazole

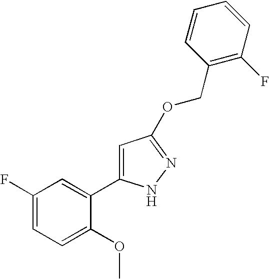

The title compound was synthesized using an analogous procedure to that described in connection with Compound 35 except that 2-methoxy-5-fluorobenzoic acid was used. ¹H NMR (400 MHz, DMSO-d₆) δ 3.85 (s, 3H) 5.23 (s, 2H) 6.28 (s, 1H) 7.05-7.26 (m, 4H) 7.39 (q, J=6.91 Hz, 1H) 7.48-7.61 (m, 2H) 12.16 (s, 1H). [M+H] calc'd for C₁₇H₁₄F₂N₂O₂ 317; found, 317.

### Compound 40: 5-(2-(difluoromethoxy)phenyl)-3-(2-fluorobenzyloxy)-1H-pyrazole

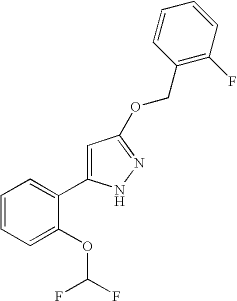

The title compound was synthesized using an analogous procedure to that described in connection with Compound 35 except that 2-difluoromethoxybenzoic acid was used. ¹H NMR (400 MHz, DMSO-d₆) δ 5.24 (s, 2H) 6.12 (d, J=2.02 Hz, 1H) 7.19-7.48 (m, 7H) 7.54-7.63 (m, 1H) 7.74 (dd, J=7.58, 1.26 Hz, 1H) 12.37 (s, 1H). [M+H] calc'd for C₁₇H₁₃F₃N₂O₂ 335; found, 335.

### Compound 41: 2-((2-(3-phenyl-1H-pyrazol-5-yl)phenoxy)methyl)pyridine

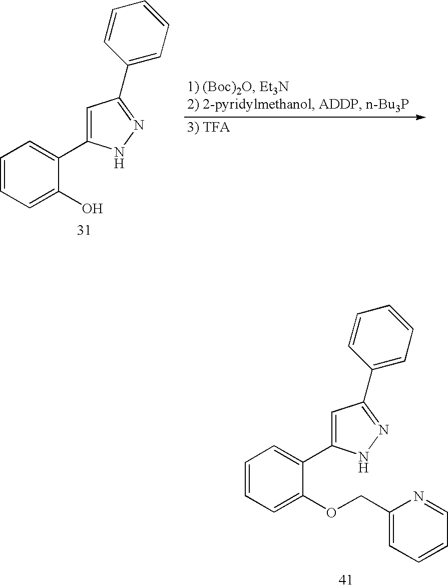

Compound 31 (610 mg) was reacted with di-tert-butyl dicarbonate (1.2 g) and triethylamine (0.8 ml) in ethanol (10 ml) at 50° C. for 15 hours. The reaction mixture was concentrated in vacuo and purified by silica gel chromatography. A solution of the obtained compound (220 mg), 2-pyridylmethanol (185 mg), ADDP (551 mg) and tri-n-butylphosphine (445 mg) was heated in toluene (5 ml) at 100° C. for 15 hours. The reaction mixture was diluted with ethyl acetate and the precipitate was removed by filtration. The filtrate was washed with aqueous NaHCO₃ solution and brine, dried over Na₂SO₄, concentrated in vacuo, and purified by silica gel chromatography. The obtained compound was dissolved in trifluoroacetic acid (1 ml) and stirred at room temperature for 1 hour. The reaction mixture was basified with aqueous NaHCO₃ solution and extracted with dichloromethane. The extract was concentrated in vacuo and purified by HPLC to give the title Compound 41 as a beige solid (190 mg). ¹H NMR (400 MHz, DMSO-d₆) δ 5.46 (s, 2H) 7.05-7.15 (m, 1H) 7.22 (d, J=8.08 Hz, 1H) 7.29-7.38 (m, 2H) 7.45 (t, J=7.33 Hz, 2H) 7.48-7.59 (m, 1H) 7.67 (d, J=7.83 Hz, 1H) 7.78-7.90 (m, 3H) 8.00 (t, J=7.83 Hz, 1H) 8.74 (d, J=4.55 Hz, 1H). [M+H] calc'd for C₂₁H₁₇N₃O 328; found, 328.

### Compound 43: (R)-6-((5-(2-methoxyphenyl)-1H-pyrazol-3-yloxy)methyl)piperazin-2-one

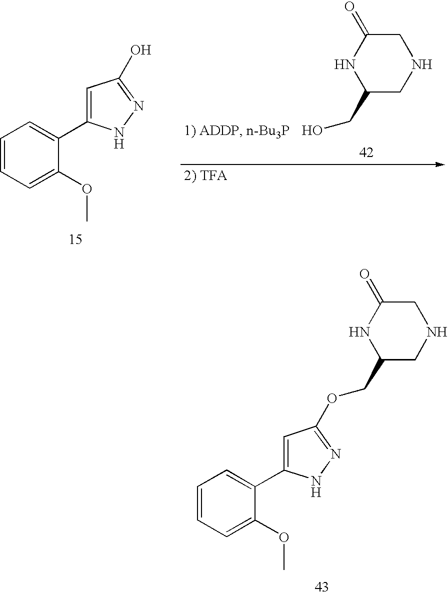

Compound 15 (190 mg) was reacted with Compound 42 (230 mg), ADDP (252 mg) and tri-n-butylphosphine (202 mg) in toluene (5 ml) at 100° C. for 15 hours. The reaction mixture was diluted with ethyl acetate and the precipitate was removed by filtration. The filtrate was washed with aqueous NaHCO₃ solution and brine, dried over Na₂SO₄, concentrated in vacuo, and purified by HPLC. The obtained compound was dissolved in trifluoroacetic acid (1 ml) and stirred at room temperature for 1 hour. The reaction mixture was basified with aqueous NaHCO₃ solution and extracted with dichloromethane. The extract was concentrated in vacuo and purified by HPLC to give the title Compound 43 as a pale beige solid (140 mg). ¹H NMR (400 MHz, DMSO-d₆) δ 3.26 (s, 1H) 3.54-3.78 (m, 3H) 3.88 (s, 3H) 4.00 (s, 1H) 4.14-4.29 (m, 2H) 6.16 (s, 1H) 7.02 (t, J=7.45 Hz, 1H) 7.13 (d, J=8.34 Hz, 1H) 7.28-7.43 (m, 1H) 7.56-7.71 (m, 1H) 8.58 (s, 1H) 9.28 (s, 1H) 12.12 (s, 1H). [M+H] calc'd for C₁₅H₁₈N₄O₃ 303; found, 303.

### Compound 44: 2-(5-(2-methoxyphenyl)-1H-pyrazol-3-yloxy) acetic acid

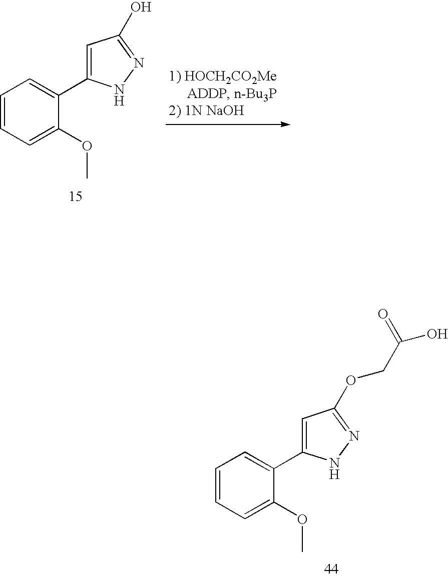

A solution of Compound 15 (190 mg), methyl glycolate (90 mg), ADDP (252 mg) and tri-n-butylphosphine (202 mg) was heated in toluene (5 ml) at 100° C. for 15 hours. The reaction mixture was diluted with ethyl acetate and the precipitate was removed by filtration. The filtrate was washed with aqueous NaHCO₃ solution and brine, dried over Na₂SO₄, and concentrated in vacuo and purified by silica gel chromatography. The obtained product was dissolved in 1N NaOH (1 ml) and ethanol (1 ml) and stirred at room temperature for 15 hours. The mixture was concentrated in vacuo and acidified with aqueous NaHSO₄, and extracted with ethyl acetate. The extract was dried over Na₂SO₄, and concentrated in vacuo. Purification by HPLC afforded the title Compound 44 as a pale colorless solid (17 mg). ¹H NMR (400 MHz, DMSO-d₆) δ 3.88 (s, 3 H) 4.67 (s, 2H) 7.12 (d, J=8.08 Hz, 1H) 7.33 (t, J=7.71 Hz, 1H) 7.63 (d, J=7.58 Hz, 1H) 12.03 (s, 1H) 12.59-13.20 (m, 1H). [M+H] calc'd for C₁₂H₁₂N₂O₄ 249; found, 249.

### Compound 45: 2-(5-(2-methoxyphenyl)-1H-pyrazol-3-yloxy)-1-morpholinoethanone

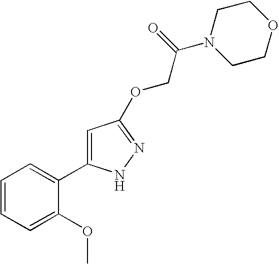

The title compound was synthesized using an analogous procedure to that described in connection with Compound 12 except that Compound 44 was used. ¹H NMR (400 MHz, METHANOL-d₄) δ 3.45 (s, 4H) 3.53-3.66 (m, 4H) 3.88 (s, 3H) 4.86 (s, 2H) 6.14 (s, 1H) 7.01 (t, J=7.45 Hz, 1H) 7.12 (d, J=8.34 Hz, 1H) 7.33 (t, J=7.71 Hz, 1H) 7.63 (d, J=7.33 Hz, 1H) 12.00 (s, 1H). [M+H] calc'd for C₁₆H₁₉N₃O₄, 318; found, 318.

### Compound 46: (5-(2-methoxyphenyl)-1H-pyrazol-3-yl)(morpholino)methanone

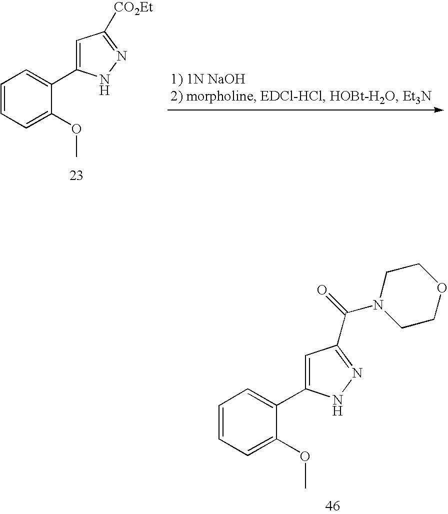

A solution of Compound 23 (246 mg) in 1N NaOH (2 ml) and ethanol (2 ml) was stirred at room temperature for 15 hours. The mixture was acidified with aqueous NaHSO₄, and extracted with ethyl acetate. The extract was dried over Na₂SO₄, and concentrated in vacuo. The obtained product was reacted with morpholine (0.02 ml), EDCI-HCl (79 mg), triethylamine (0.1 ml), and HOBt-hydrate (63 mg) in dichloromethane (3 ml) for 15 hours at room temperature. The reaction mixture was diluted with water and extracted with dichloromethane. The extract was washed with brine, dried over Na₂SO₄, and concentrated in vacuo. Purification by HPLC afforded the title Compound 46 as a colorless solid (44 mg). ¹H NMR (400 MHz, DMSO-d₆) δ 3.64 (s, 6H) 3.90 (s, 3H) 3.94-4.12 (m, 2H) 6.99 (s, 1H) 7.04 (t, J=7.45 Hz, 1H) 7.15 (d, J=8.08 Hz, 1H) 7.31-7.42 (m, 1H) 7.74 (d, J=7.83 Hz, 1H) 13.28 (s, 1H). [M+H] calc'd for C₁₅H₁₇N₃O₃ 288; found, 288.

### Compound 47: (5-(2,5-dimethoxyphenyl)-1H-pyrazol-3-yl)(morpholino)methanone

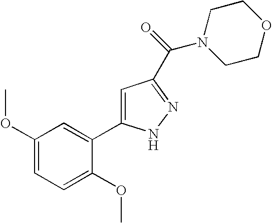

The title compound was synthesized using an analogous procedure to that described in connection with Compound 46 except that 5-(2,5-dimethoxyphenyl)-1H-pyrazole-3-carboxylic acid was used. ¹H NMR (400 MHz, DMSO-d₆) δ 3.64 (s, 6H) 3.77 (s, 3H) 3.84 (s, 3H) 3.99 (s, 2H) 6.88-6.98 (m, 1H) 6.99-7.16 (m, 2H) 7.33 (s, 1H) 12.74-13.75 (m, 1H). [M+H] calc'd for C₁₆H₁₉N₃O₄ 318; found, 318.

### Compound 48: 5-(2-methoxyphenyl)-N-phenyl-1H-pyrazole-3-carboxamide

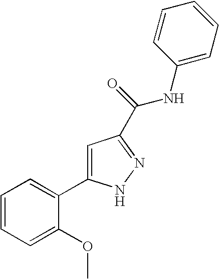

The title compound was synthesized using an analogous procedure to that described in connection with Compound 46 except that aniline was used. ¹H NMR (400 MHz, DMSO-d₆) 6 DMSO-d₆) δ 3.93 (s, 3H) 6.93-7.50 (m, 7H) 7.69-7.91 (m, 3H) 10.04 (s, 1H) 13.15-13.77 (m, 1H). [M+H] calc'd for C₁₇H₁₅N₃O₂ 294; found, 294.

### Compound 50: 3-(4-fluorophenyl)-5-(2-((1-methyl-1H-imidazol-2-yl)methoxy)phenyl)-1H-pyrazole

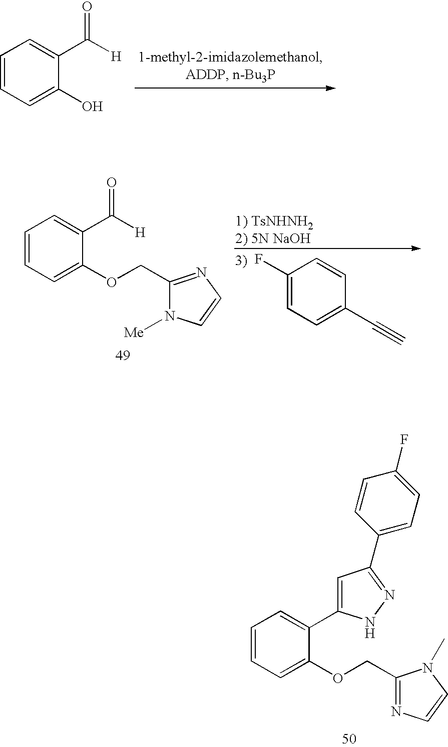

Referring to Scheme 5,2-anisaldehyde (244 mg), 1-methyl-2-imidazolemethanol (224 mg), ADDP (807 mg) and tri-n-butylphosphine (0.8 ml) were heated in toluene (10 ml) at 100° C. for 15 hours. The reaction mixture was diluted with ethyl acetate and the precipitate was removed by filtration. The filtrate was washed with aqueous NaHCO₃ solution and brine, dried over Na₂SO₄, and concentrated in vacuo. Purification by HPLC afforded the Compound 49 as beige solid. Compound 49 was reacted with tosylhydrazine (78 mg) in acetonitrile (3 ml) at room temperature for 3 hours. 5N NaOH (0.08 ml) was added and the mixture was stirred at room temperature for 15 min. Then, 4-fluorophenylacetylene (151 mg) was added and the resulting mixture was stirred at 50° C. for 48 hours. The reaction mixture was cooled to room temperature and concentrated in vacuo. The residue was diluted with water and extracted with ethyl acetate. The extract was washed with brine, dried over Na₂SO₄, and concentrated in vacuo. Purification by HPLC afforded the title Compound 50 as a beige solid (30 mg). ¹H NMR (400 MHz, DMSO-d₆) δ 3.85 (s, 3H) 5.59 (s, 2H) 7.08 (s, 1H) 7.18 (t, J=7.45 Hz, 1H) 7.23-7.34 (m, 3H) 7.36-7.45 (m, 1H) 7.68-7.73 (m, 1H) 7.74-7.79 (m, 1H) 7.80-7.89 (m, 3H). [M+H] calc'd for C₂₀H₁₇FN₄O, 349; found, 349.

### Compound 52: (3-(5-(2-methoxyphenyl)-1H-pyrazol-3-yl)phenyl)(morpholino)methanone

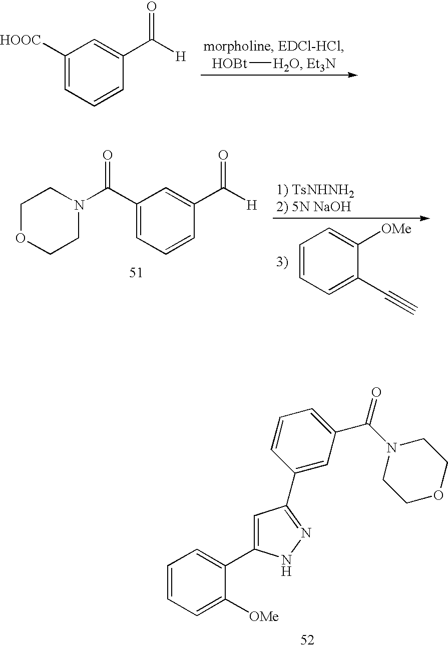

3-Formylbenzoic acid (450 mg) was reacted with morpholine (313 mg), EDCI-HCl (864 mg), triethylamine (1.3 ml), and HOBt-hydrate (689 mg) in dichloromethane (10 ml) for 15 hours at room temperature. The reaction mixture was diluted with water and extracted with dichloromethane. The extract was washed with brine, dried over Na₂SO₄, and concentrated in vacuo to give Compound 51. Compound 51 was reacted with tosylhydrazine (559 mg) in acetonitrile (5 ml) at room temperature for 3 hours. 5N NaOH (0.6 ml) was added and the mixture was stirred at room temperature for 15 min. Then, 2-methoxyphenylacetylene (0.79 g) was added and the resulting mixture was stirred at 50° C. for 48 hours. The reaction mixture was cooled to room temperature and concentrated in vacuo. The residue was diluted with water and extracted with ethyl acetate. The extract was washed with brine, dried over Na₂SO₄, and concentrated in vacuo. Purification by HPLC afforded the title Compound 52 as a pale yellow solid (87 mg). ¹H NMR (400 MHz, DMSO-d₆) δ 3.32-3.78 (m, 8H) 3.93 (s, 3H) 7.01-7.09 (m, 1H) 7.16 (d, J=7.83 Hz, 1H) 7.23 (s, 1H) 7.31-7.40 (m, 2H) 7.51 (t, J=7.71 Hz, 1H) 7.80 (dd, J=7.71, 1.64 Hz, 1H) 7.87 (s, 1H) 7.94 (d, J=8.08 Hz, 1H). [M+H] calc'd for C₂₁H₂₁N₃O₃, 364; found, 364.

### Compound 53: 3-(5-(2-methoxyphenyl)-1H-pyrazol-3-yl)benzonitrile

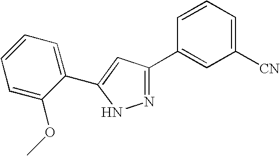

3-Formylbenzonitrile (65 mg, 0.5 mmole) was reacted with tosylhydrazine (93 mg, 0.5 mmole) in acetonitrile (3 ml) at room temperature for 3 hours. 5N NaOH (0.1 ml) was added and the mixture was stirred at room temperature for 15 min. Then, 4-fluorophenylacetylene (151 mg) was added and the resulting mixture was stirred at 50° C. for 48 hours. The reaction mixture was cooled to room temperature and concentrated in vacuo. The residue was diluted with water and extracted with ethyl acetate. The extract was washed with brine, dried over Na₂SO₄, and concentrated in vacuo. Purification by HPLC afforded the title Compound 53 as beige solid. ¹H NMR (400 MHz, DMSO-D6) δ ppm 3.92 (s, 3H) 7.01-7.09 (m, 1H) 7.16 (d, J=8.34 Hz, 1H) 7.31 (s, 1H) 7.38 (s, 1H) 7.63 (t, J=7.96 Hz, 1H) 7.71-7.79 (m, 2H) 8.17 (m, 1H) 8.20 (s, 1H) 8.27 (s, 1H). [M+H] calc'd for C₂₀H₁₇FN₄O, 275.11; found, 275.

### Compound 54: 4-(5-(2-methoxyphenyl)-1H-pyrazol-3-yl)benzonitrile

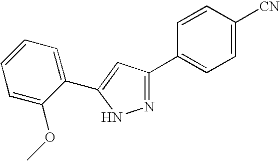

The title compound was synthesized using an analogous procedure to that described in connection with Compound 53. ¹H NMR (400 MHz, DMSO-D6) δ ppm 3.92 (s, 3H) 7.01-7.09 (t, 1H) 7.16 (d, 1H) 7.31 (s, 1H) 7.35 (s, 1H) 7.63 (t, 1H) 7.51 (d, 1H) 7.65 (d, 1H) 7.74 (d, 1H) 7.82 (d, 1H) 7.88 (d, 2H) 7.97 (m, 1H) 8.05 (d, 2H) 13.27 (s, 1H). [M+H] calc'd for C₂₀H₁₇FN₄O, 275.11; found, 275.

### Compound 55: 3-(4-fluorophenyl)-5-(2-phenoxyphenyl)-1H-pyrazole

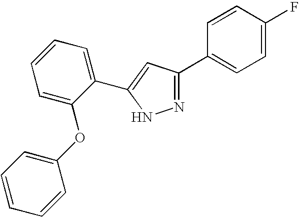

The title compound was synthesized using an analogous procedure to that described in connection with Compound 53. ¹H NMR (400 MHz, DMSO-D6) δ ppm 6.97-7.03 (m, 4H) 7.08 (t, J=7.33 Hz, 1H) 7.20-7.30 (m, 3H) 7.32-7.39 (m, 3H) 7.77 (dd, J=7.96, 5.68 Hz, 2H) 7.92 (s, 1H) 13.5-14 (br., 1H). [M+H] calc'd for C₂₁H₁₅FN₂O, 330.11; found, 330.

### Compound 56: 3-(4-fluorophenyl)-5-(3-phenoxyphenyl)-1H-pyrazole

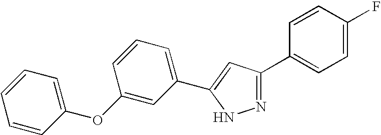

The title compound was synthesized using an analogous procedure to that described in connection with Compound 53. ¹H NMR (400 MHz, DMSO-D6) δ ppm 6.93-6.97 (m, 1H) 7.04-7.07 (m, 2H) 7.13-7.18 (m, 1H) 7.20 (s, 1H) 7.28 (t, J=8.59 Hz, 2H) 7.41 (ddd, J=8.78, 5.12, 2.02 Hz, 2H) 7.46 (t, J=7.96 Hz, 1H) 7.51 (s, 1H) 7.60 (d, J=7.83 Hz, 1H) 7.85 (dd, J=8.34, 5.56 Hz, 2H) 13.5-14 (br., 1H). [M+H] calc'd for C₂₁H₁₅FN₂O, 330.11; found, 330.

### Compound 57: 2-(3-(3-(4-fluorophenyl)-1H-pyrazol-5-yl)phenoxy)pyrimidine

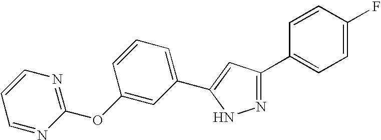

The title compound was synthesized using an analogous procedure to that described in connection with Compound 53. ¹H NMR (400 MHz, DMSO-D6) δ ppm 7.18 (s, 1H) 7.24 (s, 1H) 7.25-7.32 (m, 3H) 7.51 (t, J=7.83 Hz, 1H) 7.63-7.66 (m, 1H) 7.72 (d, J=7.33 Hz, 1H) 7.85 (dd, J=7.83, 5.81 Hz, 2H) 8.66 (s, 1H) 8.67 (s, 1H) 13.39 (s, 1H). [M+H] calc'd for C₂₁H₁₃FN₄O, 332.11; found, 332.

### Compound 58: 5-(4-(benzyloxy)-2-methoxyphenyl)-3-(4-fluorophenyl)-1H-pyrazole

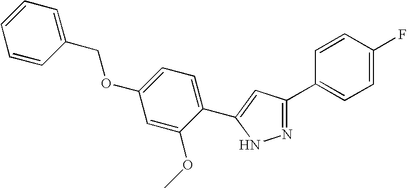

The title compound was synthesized using an analogous procedure to that described in connection with Compound 53. ¹H NMR (400 MHz, DMSO-D6) δ ppm 3.88 (s, 3H) 5.16 (s, 2H) 6.71 (s, 1H) 6.77 (s, 1H) 7.01 (s, 1H) 7.24 (s, 2H) 7.34 (s, 1H) 7.40 (s, 2H) 7.47 (s, 2H) 7.66 (s, 1H) 7.85 (s, 2H) 13.0-14.0 (br, 1H). [M+H] calc'd for C₂₃H₁₉FN₂O₂, 374.14; found, 374.

### Compound 59: 2-(3-(5-fluoro-2-methoxyphenyl)-1H-pyrazol-5-yl)pyridine

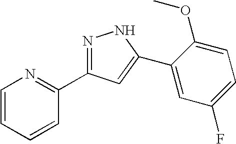

The title compound was synthesized using an analogous procedure to that described in connection with Compound 53. ¹H NMR (400 MHz, DMSO-d6) δ: 8.69 (d, J=4.0 Hz, 1H), 8.10 (m, 2H), 7.56 (d, J=8.0 Hz, 1H), 7.50 (t, J=8.0, 8.0 Hz, 1H), 7.47 (s, 1H), 7.38 (t, J=8.0, 8.0 Hz, 1H), 2.94 (s, 3H). [M+H] calc'd for C₁₅H₁₂FN₃O, 269; found, 269.

### Compound 60: 3-(3,5-difluoro-2-methoxyphenyl)-5-(4-ethynylphenyl)-1H-pyrazole

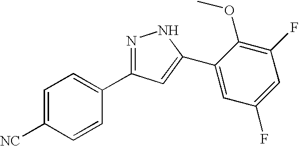

The title compound was synthesized using an analogous procedure to that described in connection with Compound 53. ¹H NMR (400 MHz, DMSO-d6) δ: 8.08 (d, J=8.0 Hz, 1H), 8.04 (d, J=12.0 Hz, 1H), 7.97 (d, J=8.0 Hz, 1H), 7.80 (s, 1H), 7.54 (m, 1H), 7.42 (m, 1H), 7.34 (m, 1H), 3.88 (d, J=4.0 Hz, 3H). [M+H] calc'd for C₁₈H₁₂F₂N₂O, 310; found, 310.

### Compound 61: 2-(3-(3-fluoro-2-methoxyphenyl)-1H-pyrazol-5-yl)pyridine

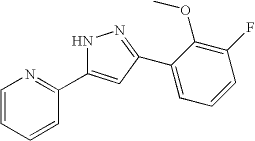

The title compound was synthesized using an analogous procedure to that described in connection with Compound 53. ¹H NMR (400 MHz, DMSO-d6) δ: 8.73 (d, J=4.0 Hz, 1H), 8.24 (m, 2H), 7.65 (m, 2H) 7.49 (s, 1H), 7.32 (m, 1H), 7.23 (m, 1H), 3.91 (s, 3H). [M+H] calc'd for C₁₅H₁₂FN₃O, 269; found, 269.

### Compound 62: 3-(5-fluoro-2-methoxyphenyl)-5-(3-fluorophenyl)-1H-pyrazole

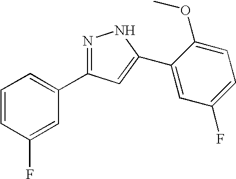

The title compound was synthesized using an analogous procedure to that described in connection with Compound 53. ¹H NMR (400 MHz, DMSO-d6) δ: 7.65 (m, 3H), 7.48 (m, 1H), 7.31 (s, 1H), 7.16 (m, 3H), 3.91 (s, 3H). [M+H] calc'd for C₁₆H₁₂F₂N₂O, 286; found, 286.

### Compound 63: 3-(3-fluoro-2-methoxyphenyl)-5-(3-fluorophenyl)-1H-pyrazole

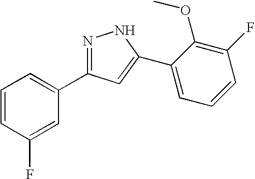

The title compound was synthesized using an analogous procedure to that described in connection with Compound 53. ¹H NMR (400 MHz, MeOH-d4) δ: 7.64 (d, J=8.0 Hz, 1H), 7.56 (m, 2H), 7.44 (m, 1H), 7.15 (m, 2H), 7.07 (m, 2H), 3.93 (s, 3H). [M+H] calc'd for C₁₆H₁₂F₂N₂O, 286; found, 286.

### Compound 64: 5-(3-fluorophenyl)-3-(2-methoxyphenyl)-1H-pyrazole

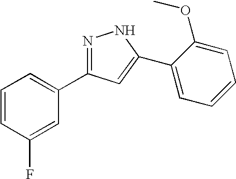

The title compound was synthesized using an analogous procedure to that described in connection with Compound 53. ¹H NMR (400 MHz, MeOH-d4) δ: 7.75 (dd, J=4.0, 8.0 Hz, 1H), 7.65 (m, 1H), 7.58 (m, 1H), 7.46-7.33 (m, 2H), 7.14 (d, J=8.0 Hz, 1H), 7.08-7.03 (m, 3H), 3.97 (s, 3H). [M+H] calc'd for C₁₆H₁₃FN₂O, 268; found, 268.

### Compound 65: 5-(4-fluorophenyl)-3-(1-methyl-1H-imidazol-2-yl)-1H-pyrazole

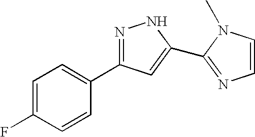

The title compound was synthesized using an analogous procedure to that described in connection with Compound 53. ¹H NMR (400 MHz, MeOH-d4) δ: 7.65-7.59 (M, 3H), 7.57-7.75 (m, 2H), 7.31 (s, 1H), 7.20 (m, 1H), 4.19 (s, 3H). [M+H] calc'd for C₁₃H₁₁FN₄, 242; found 242.

### Compound 66: 3-benzyl-5-(2-methoxyphenyl)-4H-1,2,4-triazole

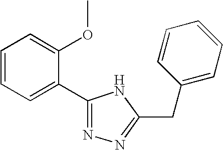

Referring to Scheme 6,2-methoxybenzonitrile (0.65 mL, 5.33 mmol) and benzylhydrazide (0.2 g, 1.33 mmol) were heated in a microwave reactor at 250° C. for one hour. Chromatography (SiO₂, 0 to 40% ethyl acetate:pentane) gave the title compound. ¹H NMR (400 MHz, DMSO-d6) δ: 11.05 (s, 1H), 7.9 (m, 1H), 7.4-7.2 (m, 6H), 6.9 (m, 2H), 4.3 (s, 2H). 3.9 (s, 3H). [M+H] calc'd for C₁₆H₁₆N₃O, 266; found 266.

### Compound 67: 3-benzyl-5-(4-tert-butylphenyl)-4H-1,2,4-triazole

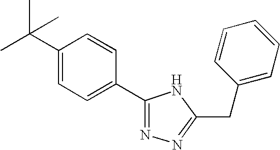

The title compound was prepared using an analogous procedure to that described in connection with Compound 66, except that 4-t-butylbenzonitrile was used instead of 2-methoxybenzonitrile. ¹H NMR (400 MHz, CDCl₃) δ: 7.89 (m, 2H), 7.48 (d, 2H), 7.31 (m, 4H), 7.23 (m, 1H), 4.09 (s, 2H), 1.30 (s, 9H). [M+H] calc'd for C₁₉H₂₂N₃, 292; found 292.

### Compound 69: N-cyclopentyl-2-(5-methyl-4H-1,2,4-triazol-3-yl)aniline

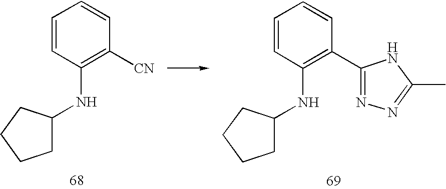

2-Fluorobenzonitrile(0.5 mL, 4.63 mmol) and cyclopentylamine (1 mL, 10.2 mmol) were heated in dimethylacetamide (5 mL) at 60° C. for four days. After cooling to RT, the reaction mixture was partitioned between ethyl acetate and brine. The organics were further washed once with brine, dried and concentrated. Chromatography (SiO₂, 5% ethyl acetate:hexanes) gave Compound 69. [M+H] calc'd for C₁₂H₁₅N₂, 161; found 161.

Compound 69 was prepared from Compound 68 using a procedure analogous to that described in connection with Compound 66, except that Compound 68 was condensed with acetic hydrazide. ¹H NMR (400 MHz, DMSO) δ: 13.7 (m, 1H), 7.95 (m, 1H), 7.19 (m, 1H), 6.73 (d, 1H), 6.59 (t, 1H), 3.89 (m, 1H), 2.40 (s, 3H), 2.01 (m, 2H), 1.70 (m, 2H), 1.61 (m, 2H), 1.46 (m, 2H). [M+H] calc'd for C₁₄H₁₉N₄, 243; found 243.

### Compound 71: 3-(2-(isopentyloxy)phenyl)-5-methyl-4H-1,2,4-triazole

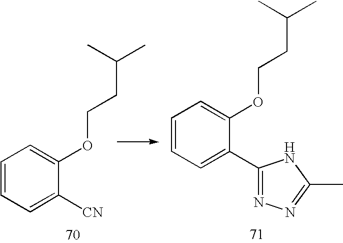

Isopentyl alcohol (6 equivalents) was treated with sodium hydride (6 eq.) in dioxane. After 10 minutes, 2-fluorobenzonitrile (1 eq.) was added and the mixture was heated to 60° C. for 30 minutes. After cooling, the reaction mixture was loaded onto a silica gel column and Compound 70 was eluted with 10% ethyl acetate:pentane.

Compound 71 was prepared using an analogous procedure to that described in connection with Compound 66, except that Compound 70 was condensed with acetic hydrazide. [M+H] calc'd for C₁₄H₂₀N₃O, 246; found 246.

### Compound 72: 3-(2-(isopentyloxy)phenyl)-5-propyl-4H-1,2,4-triazole

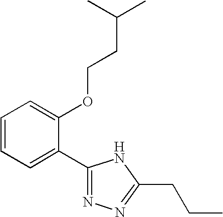

The title compound was prepared using an analogous procedure to that described in connection with Compound 66, except that Compound 70 was condensed with butyric acid hydrazide. [M+H] calc'd for C₁₆H₂₄N₃O, 274; found 274.

### Compound 73: 3-benzyl-5-(2-(isopentyloxy)phenyl)-4H-1,2,4-triazole

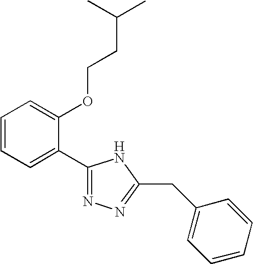

The title compound was prepared using an analogous procedure to that described in connection with Compound 66, except that Compound 70 was condensed with phenylacetic acid hydrazide. [M+H] calc'd for C₂₀H₂₄N₃O, 322; found 322.

### Compound 75: 3-benzyl-5-(2-isopropoxyphenyl)-4H-1,2,4-triazole

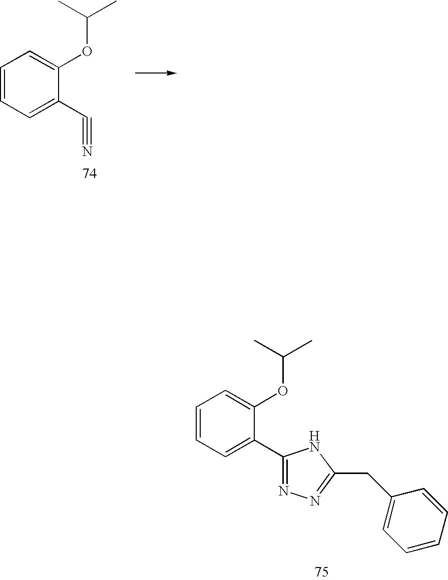

Compound 74 was prepared using an analogous procedure to that described in connection with Compound 70, except that isopropanol was reacted with 2-fluorobenzonitrile.

Compound 75 was prepared using an analogous procedure to that described in connection with Compound 66, except that Compound 74 was condensed with phenylacetic acid hydrazide. [M+H] calc'd for C₁₈H₂₀N₃O, 294; found 294.

### Compound 77: 5-(2-methoxyphenyl)-3-(trifluoromethyl)-1H-pyrazole

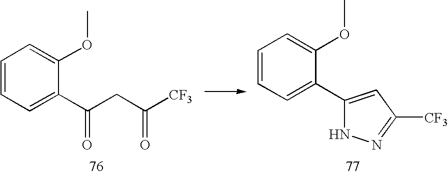

2-Methoxyacetophenone (1 mL, 7.27 mmol) was stirred in DMF (10 mL) at 0° C. Sodium hydride (240 mg, 9.45 mmol) was added. After 30 minutes, ethyl trifluoroacetate (1.13 mL, 9.45 mmol) was added and the cold bath was removed. After 4.5 hours, the mixture was poured into ice-cold 1M NaHSO₄. The organics were extracted with ether and the ether layer was subsequently washed once with brine, dried and concentrated. This crude material containing Compound 76 was used without further purification in the next reaction.

The crude material containing Compound 76 (1.69 g, 6.87 mmol) was stirred in ethanol (10 mL). Anhydrous hydrazine (5 mL) was added. The mixture was heated at 60° C. overnight. After cooling, the reaction mixture was partitioned between ethyl acetate and brine. The organics were dried and concentrated. Chromatography (SiO₂, 10 to 30% ethyl acetate:hexanes) gave Compound 77. ¹H NMR (400 MHz, CDCl₃) δ: 7.66 (dd, J=1.6, 7.7 Hz, 1H), 7.37 (m, 1H), 7.07 (m, 2H), 6.87 (s, 1H), 4.03 (s, 3H). [M+H] calc'd for C₁₁H₁₀F₃N₂O, 243; found 243.

### Compound 79: 5-(5-bromo-2-isobutoxyphenyl)-1H-pyrazole

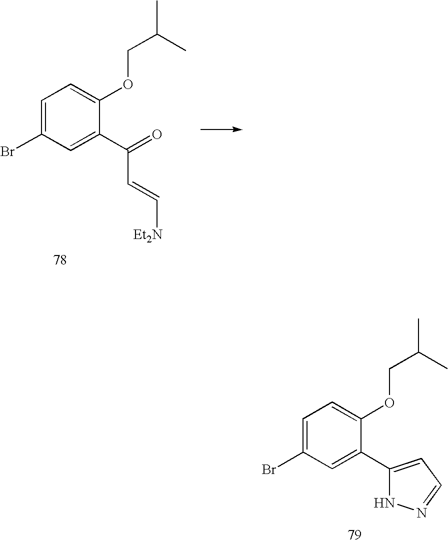

(E)-1-(5-Bromo-2-hydroxyphenyl)-3-(diethylamino)prop-2-en-1-one (524 mg, 1.76 mmol) and isobutyl bromide (0.2 mL, 1.93 mmol) were stirred in DMF (5 mL). Potassium carbonate (248 mg, 1.8 mmol) was added and the mixture stirred overnight. The reaction mixture was partitioned between ethyl acetate and 2M potassium carbonate. The organics were further washed once with brine, dried and concentrated. Chromatography (SiO₂, 20 to 40% ethyl acetate:hexanes) gave Compound 78 along with unreacted starting material. [M+H] calc'd for C₁₇H₂₅BrNO₂, 354, 356; found 354, 356.

Compound 78 (265 mg, 0.75 mmol) was stirred in ethanol (5 mL). Anhydrous hydrazine (0.12 mL, 3.7 mmol) was added. The mixture was heated at 60° C. overnight. After cooling, the reaction mixture was concentrated. Chromatography (SiO₂, 20 to 30% ethyl acetate:hexanes) gave Compound 79. ¹H NMR (400 MHz, CDCl₃) δ: 8.75 (br s, 1H), 7.83 (s, 1H), 7.69 (s, 1H), 7.38 (d, J=8.8 Hz, 1H), 6.85 (d, J=8.8 Hz, 1H), 3.86 (d, J=6.5 Hz, 2H), 2.18 (m, 1H), 1.05 (d, J=6.6 Hz, 6H). [M+H] calc'd for C₁₃H₁₆BrN2O: 295, 297; found 295, 297.

### Compound 80: (E)-5-(2-isobutoxy-5-styrylphenyl)-1H-pyrazole

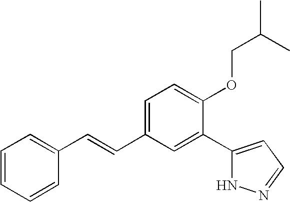

Compound 79 (507 mg, 1.72 mmol), phenylboronic acid (305 mg, 2.06 mmol), palladium tetrakistriphenylphosphine (99 mg, 0.09 mmol), cesium carbonate (2M, 5 mL) and toluene (5 mL) were stirred under nitrogen at 110° C. for 7.5 hours. After cooling, the mixture was partitioned between ethyl acetate and saturated aqueous sodium bicarbonate. The organics were dried and concentrated. Chromatography (SiO₂, 20 to 40% ethyl acetate:hexanes) gave the title compound. [M+H] calc'd for C₂₁H₂₃N₂O: 319; found 319.

### Compound 81: 5-(2-isobutoxy-5-phenethylphenyl)-1H-pyrazole

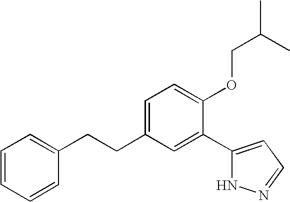

Compound 80 (170 mg, 0.54 mmol) and palladium (10% on carbon, 25 mg) were stirred in ethanol (10 mL) under hydrogen for 2.5 hours. After filtration, the organics were concentrated. Chromatography (SiO₂, 20 to 40% ethyl acetate:hexanes) gave the title compound. [M+H] calc'd for C₂₁H₂₅N₂O: 321; found 321.

### Compound 84: 5-(5-(benzyloxy)-2-(1-methoxypropan-2-yloxy)phenyl)-1H-pyrazol-3-ol

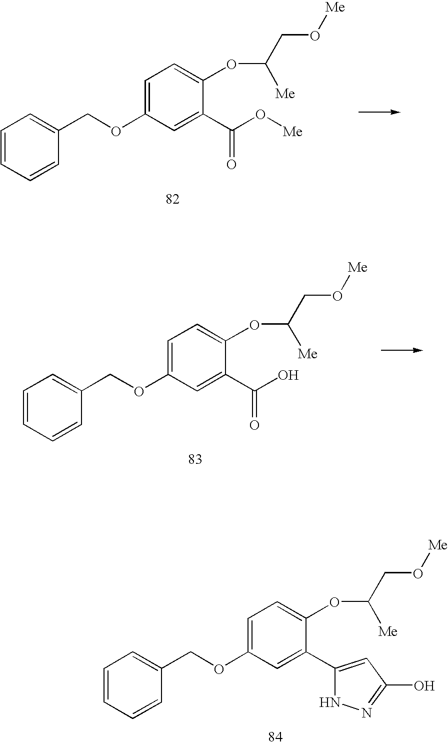

ADDP (5.85 g) was added to a mixture of methyl 5-benzyloxy-2-hydroxybenzoate (3.0 g), 1-methoxypropan-2-ol (1.05 g), tributylphosphine (4.69 g), and THF (100 mL) at room temperature. The whole was stirred at room temperature for 2 days. The precipitate was filtered off. The filtrate was concentrated in vacuo. The residue was chromatographed on SiO₂ with hexane-ethyl acetate (2/1, v/v) to give Methyl 5-(benzyloxy)-2-(1-methoxypropan-2-yloxy)benzoate (compound 82) as a colorless oil (3.20 g). ¹H NMR (400 MHz, CHLOROFORM-d) δ 1.33 (d, J=6.32 Hz, 3H), 3.42 (s, 3H), 3.46-3.68 (m, 2H), 3.90 (s, 3H), 4.38-4.49 (m, 1H), 5.06 (s, 2H), 7.00-7.12 (m, 2H), 7.31-7.50 (m, 6H).

A mixture of Compound 82 (3.0 g), 1N NaOH (20 mL), THF (20 mL), and methanol (20 mL) was stirred at 60° C. for 1 h. The mixture was washed with ether. The water layer was made acidic by 1N HCl and extracted with ethyl acetate. The organic layer was washed with brine, dried over MgSO₄, and concentrated in vacuo to give 5-(benzyloxy)-2-(1-methoxypropan-2-yloxy)benzoic acid (compound 83) as a colorless oil (2.51 g). ¹H NMR (400 MHz, CHLOROFORM-d) δ 1.46 (d, J=6.32 Hz, 3H), 3.43 (s, 3H), 3.54-3.67 (m, 2H), 4.60-4.76 (m, 1H), 5.09 (s, 2H), 7.06 (d, J=9.09 Hz, 1H), 7.18 (dd, J=9.09, 3.28 Hz, 1H), 7.31-7.50 (m, 5H), 7.77 (d, J=3.28 Hz, 1H). 4.38-4.49 (m, 1 H), 5.06 (s, 2 H), 7.00-7.12(m, 2 H), 7.31-7.50(m,6H).

A mixture of Compound 83 (0.32 g), CDI (0.19 g), and THF (5 mL) was stirred at 0° C. for 1 h and then at room temperature for 2 h. A mixture of magnesium chloride (0.13 g), ethyl malonate potassium salt (0.19 g) and THF (5 mL) was stirred at 50° C. for 2 h. The solution of CDI complex was added to the mixture of magnesium chloride and malonate potassium salt at room temperature. The whole was refluxed for 15 h. 1N HCl was added to the mixture to be acidic. The mixture was extracted with ethyl acetate. The organic layer was washed with brine, dried over MgSO₄ and concentrated in vacuo. The residue was chromatographed on SiO₂ with hexane-ethyl acetate (9/1 to 1/1, v/v) to give colorless oil. A mixture of the oil, hydrazine monohydrate (31 mg) and ethanol (5 mL) was refluxed for 5 h. Water was added to the mixture and the mixture was extracted with Ethyl acetate. The organic layer was washed with brine, dried over MgSO₄, and concentrated in vacuo. The residual crystals were washed with hexane to give compound 84 (65 mg) as colorless crystals. ¹H NMR (400 MHz, CHLOROFORM-d) δ 1.38 (d, J=6.06 Hz, 3H), 3.53-3.77 (m, 2H), 3.61 (s, 3H), 4.46-4.64 (m, 1H), 5.08 (s, 2H), 5.94 (s, 1H), 6.87-6.92 (m, 1H), 6.94-6.99 (m, 1H), 7.21 (d, J=2.78 Hz, 1H), 7.31-7.53 (m, 5H), 11.43 (br. s., 1H). [M+H] calc'd for C₂₀H₂₂N₂O₄ 355; found, 355.

### Compound 88: 5-(3-(2-fluorobenzyloxy)-5-(4-(methylsulfonyl)phenoxy)phenyl)-1H-pyrazol-3-ol

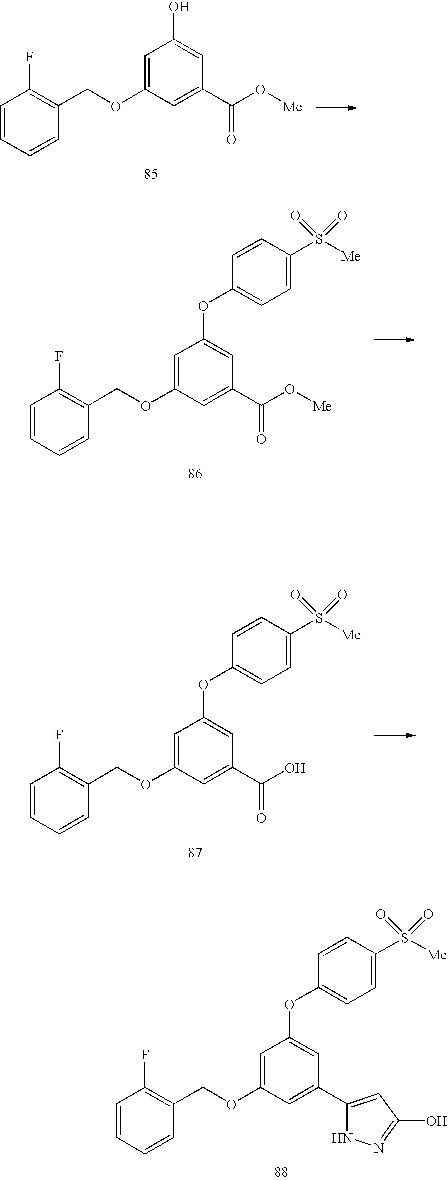

A mixture of methyl 3,5-dihydroxybenzoate (30.0 g), 2-fluorobenzylbromide (15.8 g), potassium carbonate (12.3 g) and DMF (200 mL) was stirred at room temperature for 15 h. Water was added to the mixture and the mixture was extracted with ethyl acetate. The organic layer was washed with brine, dried over MgSO₄, and concentrated in vacuo. Methylenechloride was added to the residue to give crystals. The crystals were removed by filtration. The filtrate was concentrated in vacuo. The residue was chromatographed on SiO₂ with hexane-ethyl acetate (9/1 to 1/1, v/v) to give methyl 3-(2-fluorobenzyloxy)-5-hydroxybenzoate (compound 85) as colorless crystals (17.0 g). ¹H NMR (400 MHz, CHLOROFORM-d) δ 3.93 (s, 3H), 5.15 (s, 2H), 6.69-6.76 (m, 1H), 7.04-7.39 (m, 5H), 7.51 (t, J=7.45 Hz, 1H).

A mixture of compound 85 (2.08 g), 4-bromophenyl methyl sulfone (2.12 g), (2-biphenyl)di-tert-butylphosphine (0.17 g), palladium(II) acetate (0.085 g), anhydrous potassium phosphate (3.18 g) and toluene (20 mL) was refluxed for 20 h under nitrogen atmosphere. The whole was diluted with ethyl acetate, washed successively with water and brine, dried over MgSO₄, and concentrated in vacuo. The residue was chromatographed on SiO₂ with hexane-Ethyl acetate (4/1 to 1/1, v/v) to give methyl 3-(2-fluorobenzyloxy)-5-(4-(methylsulfonyl)phenoxy)benzoate (compound 86) as colorless crystals (2.40 g). ¹H NMR (400 MHz, CHLOROFORM-d) δ 3.09 (s, 3H), 3.94 (s, 3H), 5.19 (s, 2H), 6.92 (t, J=2.27 Hz, 1H), 7.08-7.17 (m, 3H), 7.20 (t, J=7.45 Hz, 1H), 7.34-7.42 (m, 2H), 7.47-7.56 (m, 1H), 7.57 (dd, J=2.27, 1.26 Hz, 1H), 7.80-8.01 (m, 2H).

3-(2-fluorobenzyloxy)-5-(4-(methylsulfonyl)phenoxy)benzoic acid (compound 87) was synthesized using an analogous procedure to that described in connection with Compound 90 except that Compound 86 was used. Compound 87 was obtained as colorless crystals. ¹H NMR (400 MHz, DMSO-d₆) δ 3.23 (s, 3H), 5.23 (s, 2H), 7.09-7.31 (m, 6H), 7.36-7.51 (m, 2H), 7.52-7.62 (m, 1H), 7.95 (d, J=8.59 Hz, 2H).

A mixture of Compound 87 (2.18 g), CDI (1.01 g) and THF (100 mL) was refluxed for 2 h then cooled to room temperature. Magnesium chloride (0.70 g) and ethyl malonate potassium salt (0.97 g) were added to the mixture. The whole was refluxed for 15 h. 1N HCl was added to the mixture and the mixture was extracted with ethyl acetate. The organic layer was washed with brine, dried over MgSO₄, and concentrated in vacuo. The residual crystals were washed with ethyl acetate and isopropyl ether and filtered off. The filtrate was concentrated in vacuo. The residue was chromatographed on SiO₂ with hexane-ethyl acetate (2/1 to 1/4, v/v) to give colorless oil. A mixture of the oil, hydrazine monohydrate (0.24 g) and ethanol (50 mL) was refluxed for 15 h. 1N HCl was added to the mixture and the mixture was extracted with ethyl acetate. The organic layer was washed with brine, dried over MgSO₄, and concentrated in vacuo. The residue was crystallized from ethyl acetate-isopropyl ether to give the title compound 88 (1.06 g) as colorless crystals. ¹H NMR (400 MHz, DMSO-d₆) δ 3.21 (s, 3H), 5.20 (s, 2H), 6.03 (s, 1H), 6.82 (s, 1H), 7.14 (s, 1H), 7.18-7.35 (m, 5H), 7.39-7.49 (m, 1H), 7.59 (t, J=6.95 Hz, 1H), 7.91-7.96 (m, 2H). [M+H] calc'd for C₂₃H₁₉FN₂O₅S 455; found, 455.

### Compound 91: (S)-5-(3-(2-fluorobenzyloxy)-5-(1-methoxypropan-2-yloxy)phenyl)-1H-pyrazol-3-ol

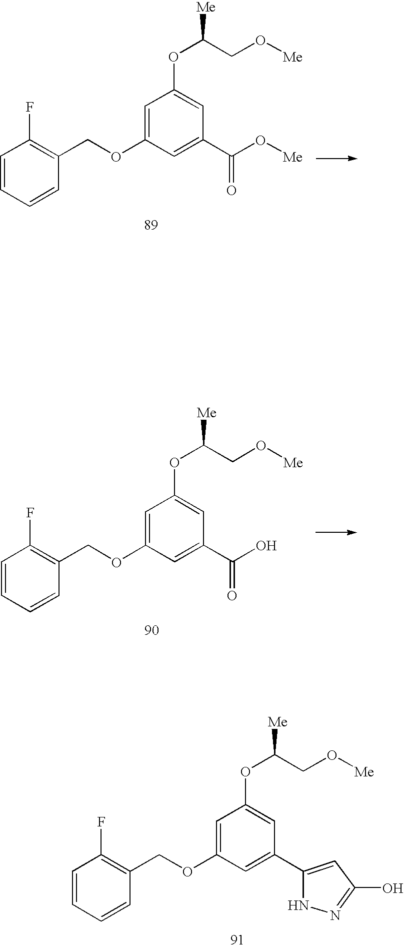

(S)-methyl 3-(2-fluorobenzyloxy)-5-(1-methoxypropan-2-yloxy)benzoate (compound 89) was synthesized using an analogous procedure to that described in connection with Compound 82 except that methyl 3-(2-fluorobenzyloxy)-5-hydroxybenzoate and (R)-1-methoxypropan-2-ol were used. ¹H NMR (400 MHz, CHLOROFORM-d) δ 1.33 (d, J=6.32 Hz, 3H), 3.43 (s, 3H), 3.48-3.64 (m, 2H), 3.92 (s, 3H), 4.53-4.66 (m, 1H), 5.16 (s, 2H), 6.80 (t, J=2.27 Hz, 1H), 7.12 (t, J=9.22 Hz, 1H), 7.19 (t, J=7.58 Hz, 1H), 7.24-7.39 (m, 3H), 7.48-7.55 (m, 1H).

A mixture of Compound 89 (7.6 g), 1N NaOH (50 mL), THF (50 mL), and methanol (50 mL) was stirred at 60° C. for 1 h. The mixture was made acidic by 1N HCl and extracted with ethyl acetate. The organic layer was washed with brine, dried over MgSO₄, and concentrated in vacuo to give (S)-3-(2-fluorobenzyloxy)-5-(1-methoxypropan-2-yloxy)benzoic acid (compound 90) as colorless crystals. ¹H NMR (400 MHz, CHLOROFORM-d) δ 1.35 (d, J=6.32 Hz, 3H), 3.45 (s, 3H), 3.50-3.66 (m, 2H), 4.57-4.67 (m, 1H), 5.17 (s, 2H), 6.85 (t, J=2.27 Hz, 1H), 7.12 (t, J=9.22 Hz, 1H), 7.20 (t, J=7.58 Hz, 1H), 7.30-7.41 (m, 3H), 7.49-7.56 (m, 1H).

Compound 91 was synthesized using an analogous procedure to that described in connection with compound 88 except that compound 90 was used. Compound 91 was obtained as colorless crystals. ¹H NMR (400 MHz, DMSO-d₆) δ 1.17-1.24 (m, 3H), 3.29 (s, 3H), 3.40-3.54 (m, 2H), 4.68 (br. s., 1H), 5.16 (s, 2H), 5.94 (br. s., 1H), 6.55 (br. s., 1H), 6.79-6.98 (m, 2H), 7.14-7.35 (m, 2H), 7.39-7.48 (m, 1H), 7.54-7.63 (m, 1H), 9.61 (br. s., 1H), 12.03 (br. s., 1H). [M+H] calc'd for C₂₀H₂₁FN₂O₅ 373; found, 373.

### Compound 94: (S)-5-(3-(1-methoxypropan-2-yloxy)-5-(4-(methylsulfonyl)phenoxy)phenyl)-1H -pyrazol-3-ol

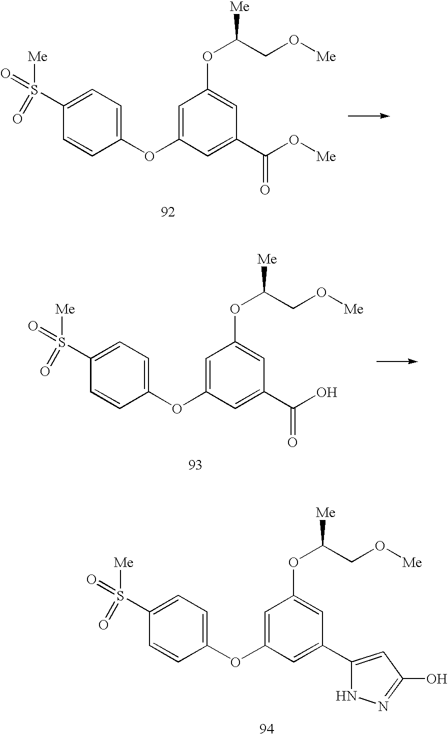

(S)-methyl 3-(1-methoxypropan-2-yloxy)-5-(4-(methylsulfonyl)phenoxy) benzoate (compound 92) was synthesized using an analogous procedure to that described in connection with Compound 82 except that methyl 3-hydroxy-5-(4-(methylsulfonyl)phenoxy)benzoate and (R)-1-methoxypropan-2-ol were used. ¹H NMR (400 MHz, CHLOROFORM-d) δ 1.35 (d, J=6.32 Hz, 3H), 3.09 (s, 3H), 3.43 (s, 3H), 3.48-3.64 (m, 2H), 3.92 (s, 3H), 4.57-4.68 (m, 1H), 6.85-6.90 (m, 1H), 7.09-7.17 (m, 2H), 7.32 (d, J=1.26 Hz, 1H), 7.48 (d, J=1.26 Hz, 1H), 7.85-7.95 (m, 2H).

A mixture of Compound 92 (4.0 g), 1N NaOH (20 mL), THF (20 mL), and methanol (20 mL) was stirred at 60° C. for 15 h. The mixture was made acidic by 1N HCl and extracted with ethyl acetate. The organic layer was washed with brine, dried over MgSO₄, and concentrated in vacuo to give (S)-3-(1-methoxypropan-2-yloxy)-5-(4-(methylsulfonyl)phenoxy)benzoic acid (compound 93) as a colorless oil. ¹H NMR (400 MHz, CHLOROFORM-d) δ 1.35 (d, J=6.32 Hz, 3H), 3.09 (s, 3H), 3.44 (s, 3H), 3.50-3.69 (m, 2H), 4.55-4.72 (m, 1H), 6.92 (t, J=2.15 Hz, 1H), 7.10-7.21 (m, 2H), 7.37 (s, 1H), 7.53 (s, 1H), 7.88-7.97 (m, 2H).

A mixture of Compound 93 (2.80 g), CDI (1.44 g) and THF (100 mL) was refluxed for 2 h then cooled to room temperature. Magnesium chloride (0.99 g) and ethyl malonate potassium salt (1.38 g) were added to the mixture. The whole was refluxed for 15 h. 1N HCl was added to the mixture and the mixture was extracted with ethyl acetate. The organic layer was washed with brine, dried over MgSO₄, and concentrated in vacuo. The residual crystals were washed with ethyl acetate and isopropyl ether and filtered off. The filtrate was concentrated in vacuo. The residue was chromatographed on SiOe with hexane-Ethyl acetate (2/1 to 1/1, v/v) to give colorless oil. A mixture of the oil, hydrazine monohydrate (0.52 g) and ethanol (50 mL) was refluxed for 15 h. Water and ethyl acetate were added to the mixture. The precipitate was collected by filtration to give the title compound 19 (1.91 g) as colorless crystals. ¹H NMR (400 MHz, DMSO-d₆) δ 1.23 (d, J=6.06 Hz, 3H), 3.21 (s, 3H), 3.29 (s, 3H), 3.42-3.54 (m, 2H), 4.71 (m., 1H), 6.00 (br. s., 1H), 6.69 (br. s., 1H), 7.05 (s, 1H), 7.17 (s, 1H), 7.19-7.26 (m, 2H), 7.87-7.98 (m, 2H), 9.65 (br. s., 1H), 12.09 (br. s., 1H). [M+H] calc'd for C₂₀H₂₂FN₂O₆S 419; found, 419.

### Compound 97: 5-(3-(cyclopentyloxy)-5-(4-(methylsulfonyl)phenoxy)phenyl)-1H-pyrazol-3-ol

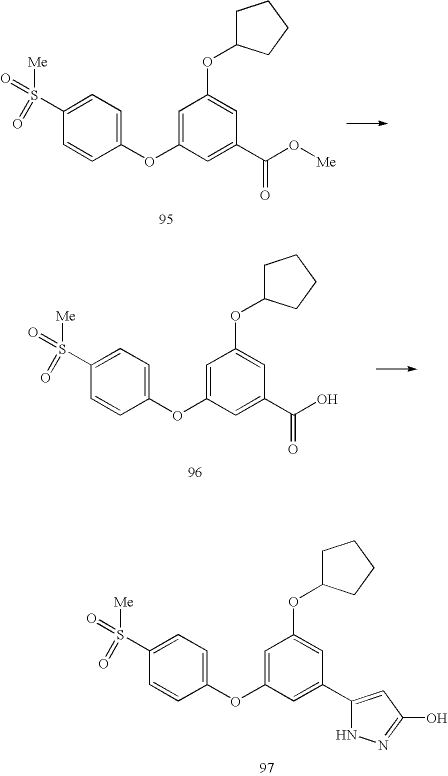

Methyl 3-(cyclopentyloxy)-5-(4-(methylsulfonyl)phenoxy)benzoate (compound 95) was synthesized using an analogous procedure to that described in connection with Compound 82 except that methyl 3-hydroxy-5-(4-(methylsulfonyl)phenoxy)benzoate and cyclopentanol were used. ¹H NMR (400 MHz, CHLOROFORM-d) δ 1.60-2.04 (m, 8H), 3.09 (s, 3H), 3.92 (s, 3H), 4.77-4.86 (m, 1H), 6.80 (t, J=2.27 Hz, 1H), 7.06-7.18 (m, 2H), 7.25-7.30 (m, 1H), 7.40-7.45 (m, 1H), 7.86-7.97 (m, 2H).

3-(cyclopentyloxy)-5-(4-(methylsulfonyl)phenoxy)benzoic acid (compound 96) was synthesized using an analogous procedure to that described in connection with Compound 90 except that Compound 95 was used. Compound 96 was obtained as colorless crystals. ¹H NMR (400 MHz, CHLOROFORM-d) δ 1.57-2.03 (m, 8H), 3.09 (s, 3H), 4.79-4.87 (m, 1H), 6.85 (t, J=2.27 Hz, 1H), 7.10-7.21 (m, 2H), 7.35 (dd, J=2.27, 1.26 Hz, 1H), 7.44-7.50 (m, 1H), 7.84-8.00(m, 2H).

Compound 97 was synthesized using an analogous procedure to that described in connection with Compound 94 except that Compound 96 was used. The title compound was obtained as colorless crystals. ¹H NMR (400 MHz, DMSO-d₆) δ 1.52-2.01 (m, 8H), 3.21 (s, 3H), 4.84-4.93 (m, 1H), 5.98 (br. s., 1H), 6.62 (br. s., 1H), 7.04 (s, 1H), 7.12 (s, 1H), 7.17-7.26 (m, 2H), 7.89-7.95 (m, 2H), 9.66 (br. s., 1H), 12.10 (br. s., 1H). [M+H] calc'd for C₂₁H₂₂N₂O₅S 414; found, 414.

### Compound 100: 5-(3-((3-methylpyridin-2-yl)methoxy)-5-(4-(methylsulfonyl)phenoxy)phenyl)-1H-pyrazol-3-ol

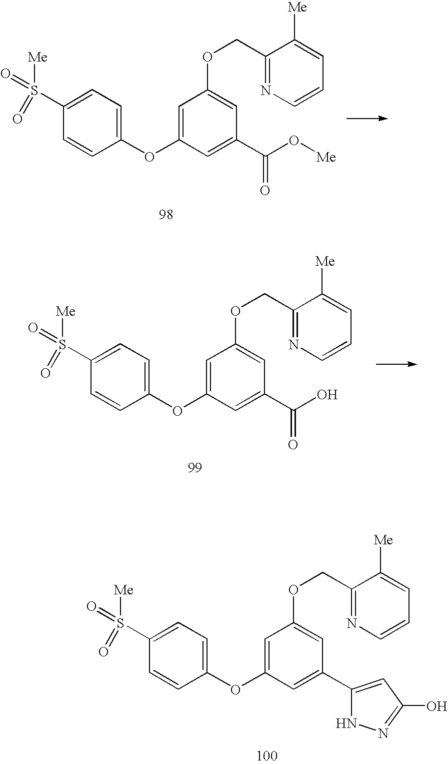

A mixture of methyl 3-hydroxy-5-(4-(methylsulfonyl)phenoxy)benzoate (0.50 g), 2-chloromethyl-3-methylpyridine (0.27 g), potassium carbonate (0.22 g), and DMF (10 mL) was stirred at 80° C. for 15 h. Water was added to the mixture and the mixture was extracted with ethyl acetate. The organic layer was washed with brine, dried over MgSO₄, and concentrated in vacuo. The residue was chromatographed on SiO₂ with hexane-Ethyl acetate (1/1 to 1/9, v/v) to give methyl 3-((3-methylpyridin-2-yl)methoxy)-5-(4-(methylsulfonyl)phenoxy) benzoate (compound 98). ¹H NMR (400 MHz, CHLOROFORM-d) δ 2.45 (s, 3H), 3.09 (s, 3H), 3.93 (s, 3H), 5.26 (s, 2H), 6.98 (t, J=2.40 Hz, 1H), 7.06-7.15 (m, 2H), 7.23 (dd, J=7.58, 4.80 Hz, 1H), 7.33-7.36 (m, 1H), 7.49-7.63 (m, 2H), 7.87-7.99 (m, 2H), 8.41-8.48 (m, 1H).

A mixture of compound 98 (0.67 g), 1N NaOH (5 mL), THF (5 mL), and methanol (5 mL) was refluxed for 15 h. 1N HCl (5 mL) was added to the mixture. The precipitate was collected by filtration to give 3-((3-methylpyridin-2-yl)methoxy)-5-(4-(methylsulfonyl)phenoxy)benzoic acid (compound 99) as colorless crystals (0.52 g). ¹H NMR (400 MHz, DMSO-d₆) δ 2.38 (s, 3H), 3.23 (s, 3H), 5.28 (s, 2H), 7.11-7.17 (m, 2H), 7.19-7.27 (m, 2H), 7.31 (dd, J=7.58, 4.80 Hz, 1H), 7.42-7.45 (m, 1H), 7.66 (d, J=7.58 Hz, 1H), 7.86-7.98 (m, 2H), 8.37 (d, J=3.79 Hz, 1H), 13.28 (br. s., 1H).

Compound 100 was synthesized using an analogous procedure to that described in connection with Compound 94 except that Compound 99 was used. Compound 100 was obtained as colorless crystals. ¹H NMR (400 MHz, DMSO-d₆) δ 2.38 (s, 3H), 3.21 (s, 3H), 5.25 (s, 2H), 5.99 (br. s., 1H), 6.80 (br. s., 1H), 7.09 (br. s., 1H), 7.16-7.23 (m, 2H), 7.27 (s, 1H), 7.32 (dd, J=7.58, 4.80 Hz, 1H), 7.67 (d, J=7.07 Hz, 1H), 7.84-8.01 (m, 2H), 8.38 (d, J=3.54 Hz, 1H), 9.66 (br. s., 1H), 12.11 (br. s., 1H). [M+H] calc'd for C₂₃H₂₁N₃O₅S 452; found, 452.

### Compound 103: 5-(3-(2-methylbenzyloxy)-5-(4-(methylsulfonyl)phenoxy)phenyl)-1H-pyrazol-3-ol

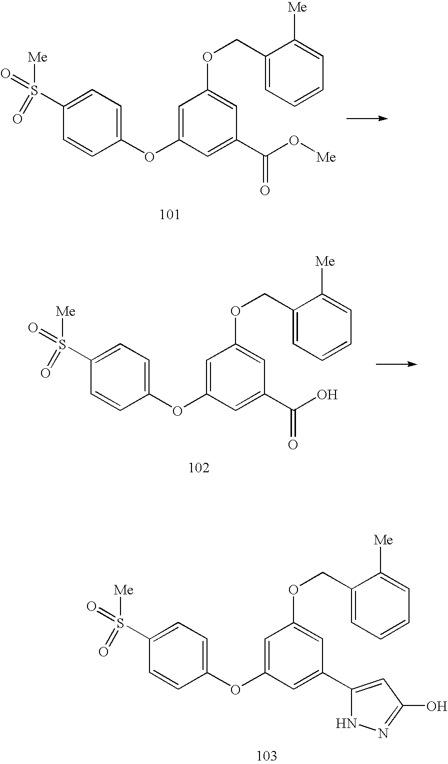

Methyl 3-(2-methylbenzyloxy)-5-(4-(methylsulfonyl)phenoxy)benzoate (compound 101) was synthesized using an analogous procedure to that described in connection with compound 98 except that 2-methylbenzylbromide was used. ¹H NMR (400 MHz, CHLOROFORM-d) δ 2.40 (s, 3H), 3.09 (s, 3H), 3.94 (s, 3H), 5.10 (s, 2H), 6.91 (t, J=2.15 Hz, 1H), 7.09-7.17 (m, 2H), 7.19-7.46 (m, 5H), 7.57 (s, 1H), 7.85-7.98 (m, 2H).

3-(2-methylbenzyloxy)-5-(4-(methylsulfonyl)phenoxy)benzoic acid (compound 102) was synthesized using an analogous procedure to that described in connection with Compound 90 except that Compound 101 was used. Compound 102 was obtained as colorless crystals. ¹H NMR (400 MHz, CHLOROFORM-d) δ 2.41 (s, 3H), 3.10 (s, 3H), 5.12 (s, 2H), 6.96 (t, J=2.27 Hz, 1H), 7.12-7.18 (m, 2H), 7.20-7.45 (m, 5H), 7.61 (s, 1H), 7.86-8.05 (m, 2H)

Compound 103 was synthesized using an analogous procedure to that described in connection with compound 94 except that compound 102 was used. Compound 103 was obtained as pale yellow crystals. ¹H NMR (400 MHz, DMSO-d₆) δ 2.34 (s, 3H), 3.21 (s, 3H), 5.15 (s, 2H), 5.99 (br. s., 1H), 6.81 (br. s., 1H), 7.10 (s, 1H), 7.16-7.36 (m, 6H), 7.43 (d, J=7.07 Hz, 1H), 7.88-7.98 (m, 2H), 9.67 (br. s., 1H), 12.14 (br. s., 1H). [M+H] calc'd for C₂₄H₂₂FN₂O₅S 451; found, 451.

### Compound 104: 5-(5-(benzyloxy)-2-(1-methoxypropan-2-yloxy)phenyl)-3-methoxy-1H-pyrazole

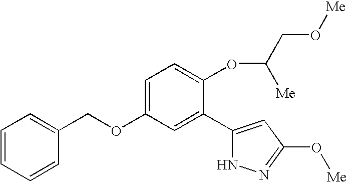

ADDP (1.41 g) was added to a mixture of Compound 84 (1.0 g), methanol (0.27 g), tributylphosphine (1.13 g), and toluene (100 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was chromatographed on SiO₂ with hexane-Ethyl acetate (9/1 to 1/1, v/v) to give the title compound 104 as colorless crystals (3.20 g). ¹H NMR (400 MHz, CHLOROFORM-d) δ 1.39 (d, J=6.32 Hz, 3H), 3.51 (s, 3H), 3.56-3.71 (m, 2H), 3.96 (s, 3H), 4.50-4.63 (m, 1H), 5.07 (s, 2H), 5.97 (s, 1H), 6.86-6.91 (m, 1H), 6.95-6.99 (m, 1H), 7.19 (d, J=3.03 Hz, 1H), 7.32-7.50 (m, 5H), 11.13 (br. s., 1H). [M+H] calc'd for C₂₁H₂₄N₂O₄ 369; found, 369.

### Compound 105: (S)-5-(3-(2-fluorobenzyloxy)-5-(1-methoxypropan-2-yloxy)phenyl)-3-methoxy-1H-pyrazole

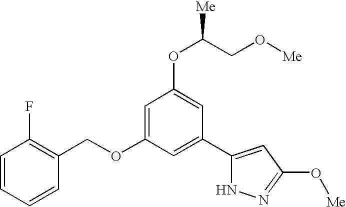

The title compound was synthesized using an analogous procedure to that described in connection with Compound 104 except that Compound 91 was used. The title compound was obtained as a colorless oil. ¹H NMR (400 MHz, CHLOROFORM-d) δ 1.33 (d, J=6.06 Hz, 3H), 3.43 (s, 3H), 3.47-3.65 (m, 2H), 3.95 (s, 3H), 4.48-4.70 (m, 1H), 5.15 (s, 2H), 5.95 (s, 1H), 6.58-6.63 (m, 1H), 6.75 (d, J=6.82 Hz, 2H), 7.12 (t, J=9.22 Hz, 1H), 7.19 (t, J=7.07 Hz, 1H), 7.30-7.43 (m, 1H), 7.48-7.56 (m, 1H). [M+H] calc'd for C₂₁H₂₃FN₂O₄ 387; found, 387.

### Compound 106: 5-(3-(2-fluorobenzyloxy)-5-(4-(methylsulfonyl)phenoxy)phenyl)-3-methoxy-1H -pyrazole

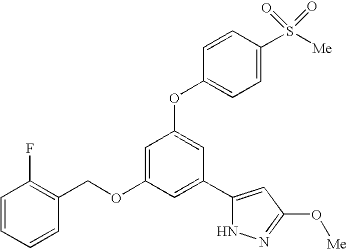

ADDP (0.91 g) was added to a mixture of Compound 88 (0.80 g), methanol (0.12 g), tributylphosphine (0.73 g), and 1,4-dioxane (30 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was chromatographed on SiO₂ with hexane-ethyl acetate (3/1 to 1/9, v/v) to give the title compound 106 as colorless crystals (3.20 g). ¹H NMR (400 MHz, DMSO-d₆) δ 3.21 (s, 3H), 3.79 (s, 3H), 5.20 (s, 2H), 6.29 (s, 1H), 6.84 (s, 1H), 7.13-7.38 (m, 6H), 7.41-7.49 (m, 1H), 7.53-7.66 (m, 1H), 7.91-8.00 (m, 2H), 12.40 (br. s., 1H). [M+H] calc'd for C₂₄H₂₁FN₂O₅S 469; found, 469.

### Compound 107: 5-(3-(cyclopentyloxy)-5-(4-(methylsulfonyl)phenoxy)phenyl)-3-methoxy-1H-pyrazole

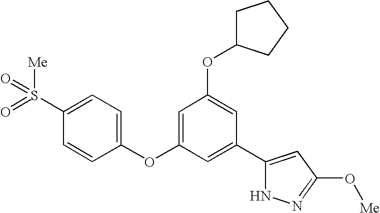

ADDP (0.21 g) was added to a mixture of Compound 97 (0.18 g), methanol (0.02 g), tributylphosphine (0.17 g), and 1,4-dioxane (10 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was chromatographed on SiO₂ with hexane-ethyl acetate (1/2 to 1/9 v/v) to give the title compound as a colorless oil (60 mg). ¹H NMR (400 MHz, CHLOROFORM-d) δ 1.57-2.02 (m, 8H), 3.08 (s, 3H), 3.91 (s, 3H), 4.68-4.82 (m, 1H), 5.93 (s, 1H), 6.58 (t, J=2.15 Hz, 1H), 6.81-6.86 (m, 1H), 6.94 (s, 1H), 7.10-7.16 (m, 2H), 7.86-7.94 (m, 2H). [M+H] calc'd for C₂₂H₂₄N₂O₅S 429; found, 429.

### Compound 108: 2-((3-(3-methoxy-1H-pyrazol-5-yl)-5-(4-(methylsulfonyl)phenoxy)phenoxy)methyl)-3-methylpyridine

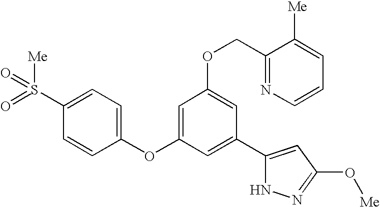

ADDP (0.27 g) was added to a mixture of Compound 100 (0.24 g), methanol (0.026 g), tributylphosphine (0.21 g), and 1,4-dioxane (30 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was chromatographed on SiO₂ with hexane-ethyl acetate (1/4 to 5/95, v/v) to give the title compound as colorless crystals (100 mg). ¹H NMR (400 MHz, CHLOROFORM-d) δ 2.45 (s, 3H), 3.08 (s, 3H), 3.93 (s, 3H), 5.26 (s, 2H), 5.94 (s, 1H), 6.71 (t, J=2.15 Hz, 1H), 6.87 (s, 1H), 7.05-7.13 (m, 2H), 7.17 (s, 1H), 7.23 (dd, J=7.58, 4.80 Hz, 1H), 7.56 (d, J=7.33 Hz, 1H), 7.86-7.93 (m, 2H), 8.39-8.46 (m, 1H). [M+H] calc'd for C₂₄H₂₃N₃O₅S 466; found, 466.

### Compound 109: 3-methoxy-5-(3-(2-methylbenzyloxy)-5-(4-(methylsulfonyl)phenoxy)phenyl)-1H -pyrazole

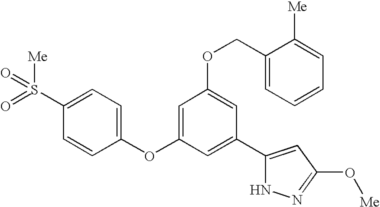

The title compound was synthesized using an analogous procedure to that described in connection with Compound 108 except that Compound 103 was used. The title compound was obtained as a colorless oil. ¹H NMR (400 MHz, CHLOROFORM-d) δ 2.39 (s, 3H), 3.08 (s, 3H), 3.92 (s, 3H), 5.07 (s, 2H), 5.95 (s, 1H), 6.69 (t, J=2.15 Hz, 1H), 6.89 (s, 1H), 7.07 (s, 1H), 7.19-7.34 (m, 5H), 7.39 (d, J=7.33 Hz, 1H), 7.88-7.98 (m, 2H), 9.26-9.89 (m, 1H). [M+H] calc'd for C₂₅H₂₄N₂O₅S 465; found, 465.

### Compound 110: 5-(2-(5-(5-(benzyloxy)-2-(1-methoxypropan-2-yloxy)phenyl)-1H-pyrazol-3-yloxy)ethyl)-4-methylthiazole

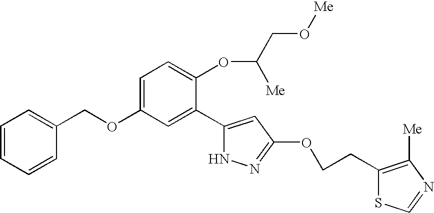

ADDP (0.14 g) was added to a mixture of Compound 84 (0.10 g), 2-(4-methylthiazol-5-yl)ethanol (0.06 g), tributylphosphine (0.11 g), and toluene (10 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was purified with chromatography on SiO₂ with hexane-ethyl acetate (1/1 to 1/5, v/v) and then purified with HPLC to give the title compound as a colorless oil (0.04 g). ¹H NMR (400 MHz, CHLOROFORM-d) δ 1.39 (d, J=6.32 Hz, 3H), 2.47 (s, 3H), 3.28 (t, J=6.57 Hz, 2H), 3.51 (s, 3H), 3.56-3.70 (m, 2H), 4.39 (t, J=6.69 Hz, 2H), 4.49-4.63 (m, 1H), 5.07 (s, 2H), 5.97 (s, 1H), 6.86-6.92 (m, 1H), 6.94-7.02 (m, 1H), 7.18 (d, J=2.78 Hz, 1H), 7.32-7.49 (m, 5H), 8.61 (s, 1H), 11.20 (br. s., 1H). [M+H] calc'd for C₂₆H₂₉N₃O₄S 480; found, 480.

### Compound 111: (S)-5-(2-(5-(3-(2-fluorobenzyloxy)-5-(1-methoxypropan-2-yloxy)phenyl)-1H-pyrazol-3-yloxy)ethyl)-4-methylthiazole

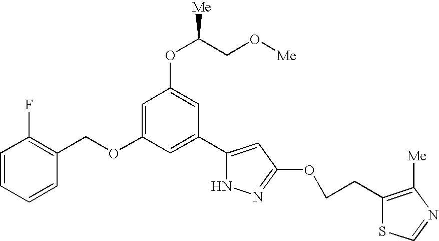

ADDP (0.14 g) was added to a mixture of Compound 91 (0.10 g), 2-(4-methylthiazol-5-yl)ethanol (0.046 g), tributylphosphine (0.11 g), and toluene (5 mL) at 70° C. The whole was stirred at 70° C. for 5 h, then concentrated in vacuo. Isopropyl ether was added to the mixture and the insoluble materials were removed by filtration. The filtrate was concentrated in vacuo. The residue was purified with chromatography on SiO₂ with hexane-Ethyl acetate (4/1 to 1/2, v/v) to give crystals. The crystals were recrystallized from ethyl acetate-hexane to give the title compound as colorless prisms (0.09 g). ¹H NMR (400 MHz, CHLOROFORM-d) δ 1.33 (d, J=6.06 Hz, 3H), 2.46 (s, 3H), 3.27 (t, J=6.44 Hz, 2H), 3.43 (s, 3H), 3.47-3.66 (m, 2H), 4.38 (t, J=6.57 Hz, 2H), 4.54-4.65 (m, 1H), 5.15 (s, 2H), 5.95 (s, 1H), 6.56-6.64 (m, 1H), 6.69-6.80 (m, 2H), 7.07-7.24 (m, 2H), 7.26-7.43 (m, 1H), 7.47-7.58 (m, 1H), 8.61 (s, 1H), 9.30 (br. s., 1H). [M+H] calc'd for C₂₆H₂₈FN₃O₄S 498; found, 498.

### Compound 112: (S)-methyl 2-(5-(3-(1-methoxypropan-2-yloxy)-5-(4-(methylsulfonyl)phenoxy)phenyl)-1H-pyrazol-3-yloxy)acetate

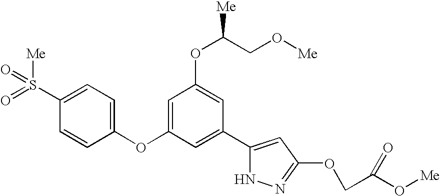

ADDP (0.35 g) was added to a mixture of Compound 94 (0.30 g), methyl glycolate, tributylphosphine (0.28 g), and 1,4-dioxane (10 mL) at 80° C. The whole was stirred at 80° C. for 3 h, then concentrated in vacuo. Isopropyl ether was added to the mixture and the insoluble materials were removed by filtration. The filtrate was concentrated in vacuo. The residue was purified with chromatography on SiO₂ with hexane-Ethyl acetate (1/1 to 1/5, v/v) to give the title compound as a colorless oil (0.13 g). ¹H NMR (400 MHz, CHLOROFORM-d) δ 1.34 (d, J=6.32 Hz, 3H), 3.09 (s, 3H), 3.42 (s, 3H), 3.49-3.65 (m, 2H), 3.82 (s, 3H), 4.52-4.63 (m, 1H), 4.83 (s, 2H), 6.02 (s, 1H), 6.65 (t, J=2.15 Hz, 1H), 6.77-6.82 (m, 1H), 6.94-6.98 (m, 1H), 7.11-7.20 (m, 2H), 7.84-7.95 (m, 2H). [M+H] calc'd for C₂₃H₂₆N₂O₈S 491; found, 491.

### Compound 113: 3-(3-methoxy-1H-pyrazol-5-yl)-4-(1-methoxypropan-2-yloxy)phenol

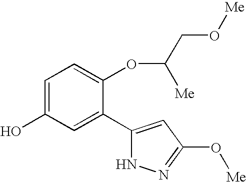

A mixture of compound 104 (1.60 g), 10% Pd—C (dry, 300 mg) and methanol (20 mL) was hydrogenated under atmospheric pressure. The catalyst was removed by filtration. The filtrate was concentrated in vacuo. The residue was chromatographed on SiO₂ with hexane-ethyl acetate (3/2 to 1/1, v/v) to give the title compound as a colorless oil (0.95 g). ¹H NMR (400 MHz, CHLOROFORM-d) δ 1.36 (d, J=6.32 Hz, 3H), 3.52 (s, 3H), 3.55-3.69 (m, 2H), 3.97 (s, 3H), 4.48-4.60 (m, 1H), 5.99 (s, 1H), 6.81 (dd, J=8.84, 3.03 Hz, 1H), 6.92 (d, J=9.09 Hz, 1H), 7.09 (d, J=2.78 Hz, 1H). [M+H] calc'd for C₁₄H₁₈N₂O₄ 279; found, 279.

### Compound 114: 3-(3-methoxy-1H-pyrazol-5-yl)-5-(4-(methylsulfonyl)phenoxy)phenol

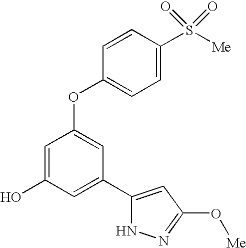

A mixture of compound 106 (0.12 g), 10% Pd—C (dry, 100 mg) and THF (20 mL) was hydrogenated under atmospheric pressure. The catalyst was removed by filtration. The filtrate was concentrated in vacuo. The residue was chromatographed on SiO₂ with hexane-ethyl acetate (1/1 to 1/4, v/v) to give the title compound as colorless crystals (0.05 g). ¹H NMR (400 MHz, DMSO-d₆) δ 3.21 (s, 3H), 3.78 (s, 3H), 6.14 (d, J=1.77 Hz, 1H), 6.47 (s, 1H), 6.98 (d, J=5.56 Hz, 2H), 7.11-7.31 (m, 2H), 7.81-7.96 (m, 2H), 10.00 (s, 1H), 12.34 (d, J=1.26 Hz, 1H). [M+H] calc'd for C₁₇H₁₆N₂O₅S 3615; found, 361.

### Compound 115: (S)-3-(3-methoxy-1H-pyrazol-5-yl)-5-(1-methoxypropan-2-yloxy)phenol

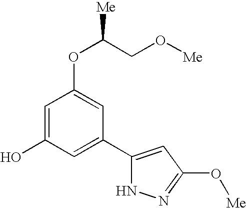

A mixture of compound 105 (0.73 g), 10% Pd—C (dry, 200 mg) and methanol (50 mL) was hydrogenated under atmospheric pressure. The catalyst was removed by filtration. The filtrate was concentrated in vacuo. The residue was chromatographed on SiO₂ with hexane-ethyl acetate (1/1 to 1/4, v/v) to give the title compound as a colorless oil (0.45 g). ¹H NMR (400 MHz, CHLOROFORM-d) δ 1.27-1.29 (m, 3H), 3.42 (s, 3H), 3.44-3.66 (m, 2H), 3.89 (s, 3H), 4.45-4.60 (m, 1H), 5.84 (s, 1H), 6.47 (s, 1H), 6.65 (s, 1H), 6.74 (s, 1H). [M+H] calc'd for C₁₄H₁₈N₂O₄ 279; found, 279.

### Compound 116: 5-(5-ethoxy-2-(1-methoxypropan-2-yloxy)phenyl)-3-methoxy-1H-pyrazole

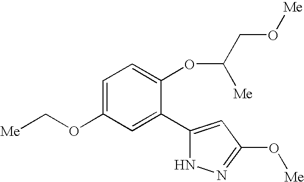

ADDP (0.20 g) was added to a mixture of Compound 113 (0.15 g), ethanol (0.075 g), tributylphosphine (0.16 g), and toluene (10 mL) at 70° C. The whole was stirred at 70° C. for 1 h, then concentrated in vacuo. Isopropyl ether was added to the mixture and the insoluble materials were removed by filtration. The filtrate was concentrated in vacuo. The residue was chromatographed on SiO₂ with hexane-ethyl acetate (9/1 to 1/1, v/v) to give the title compound as colorless oil (0.11 g). ¹H NMR (400 MHz, CHLOROFORM-d) δ 1.37 (d, J=6.32 Hz, 3H), 1.43 (t, J=6.95 Hz, 3H), 3.50 (s, 3H), 3.55-3.68 (m, 2H), 3.96 (s, 3H), 3.99-4.08 (m, 2H), 4.53-4.63 (m, 1H), 5.99 (s, 1H), 6.81 (dd, J=8.84, 3.03 Hz, 1H), 6.96 (d, J=9.09 Hz, 1H), 7.10 (d, J=3.03 Hz, 1H). [M+H] calc'd for C₁₆H₂₂N₂O₄ 307; found, 307.

### Compound 117: 3-methoxy-5-(2-(1-methoxypropan-2-yloxy)-5-(2-(thiophen-3-yl)ethoxy)phenyl)-1H-pyrazole

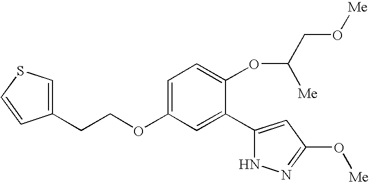

ADDP (0.30 g) was added to a mixture of Compound 113 (0.22 g), 2-(thiophen-3-yl)ethanol (0.10 g), tributylphosphine (0.24 g), and toluene (10 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was purified by chromatographed on SiO₂ with hexane-Ethyl acetate (2/1 to 1/2, v/v) then by HPLC to give the title compound as a colorless oil (0.15 g). ¹H NMR (400 MHz, CHLOROFORM-d) δ 1.38 (d, J=6.32 Hz, 3H), 3.15 (t, J=6.82 Hz, 2H), 3.51 (s, 3H), 3.57-3.69 (m, 2H), 3.96 (s, 3H), 4.19 (t, J=6.82 Hz, 2H), 4.49-4.64 (m, 1H), 5.99 (s, 1H), 6.82 (dd, J=8.84, 3.03 Hz, 1H), 6.97 (d, J=9.09 Hz, 1H), 7.04-7.15 (m, 3H), 7.31 (dd, J=5.05, 3.03 Hz, 1H), 11.13 (br. s., 1H). [M+H] calc'd for C₂₀H₂₄N₂O₄S 389; found, 389.

### Compound 118: 3-methoxy-5-(2-(1-methoxypropan-2-yloxy)-5-(4-(methylsulfonyl)benzyloxy)phenyl)-1H-pyrazole

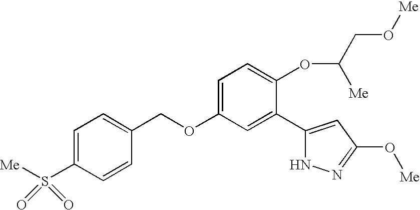

ADDP (0.20 g) was added to a mixture of Compound 113 (0.15 g), (4-(methylsulfonyl)phenyl)methanol (0.10 g), tributylphosphine (0.16 g), and toluene (10 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was purified by chromatographed on SiO₂ with hexane-ethyl acetate (1/1 to 1/4, v/v) then by HPLC to give the title compound as a colorless oil (0.05 g). ¹H NMR (400 MHz, CHLOROFORM-d) δ 1.39 (d, J=6.32 Hz, 3H), 3.09 (s, 3H), 3.51 (s, 3H), 3.56-3.71 (m, 2H), 3.96 (s, 3H), 4.53-4.65 (m, 1H), 5.17 (s, 2H), 5.97 (s, 1H), 6.87 (dd, J=8.97, 2.91 Hz, 1H), 6.99 (d, J=9.09 Hz, 1H), 7.18 (d, J=3.03 Hz, 1H), 7.65-7.71 (m, 2H), 7.97-8.02 (m, 2H), 11.15 (br. s., 1H). [M+H] calc'd for C₂₂H₂₆N₂O₆S 447; found, 447.

### Compound 119: 4-((3-(3-methoxy-1H-pyrazol-5-yl)-4-(1-methoxypropan-2-yloxy)phenoxy)methyl)pyridine

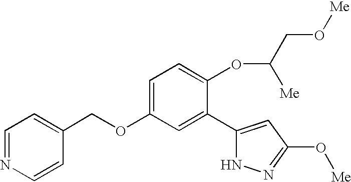

ADDP (0.20 g) was added to a mixture of Compound 113 (0.15 g), pyridin-4-ylmethanol (0.06 g), tributylphosphine (0.16 g), and toluene (10 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was purified by chromatographed on SiO₂ with hexane-ethyl acetate (1/4 to 1/9, v/v) then by HPLC to give the title compound as a colorless oil (0.06 g). ¹H NMR (400 MHz, CHLOROFORM-d) δ 1.39 (d, J=6.32 Hz, 3H), 3.51 (s, 3H), 3.57-3.70 (m, 2H), 3.94-3.97 (m, 3H), 4.55-4.66 (m, 1H), 5.10 (s, 2H), 5.97 (s, 1H), 6.86 (dd, J=8.84, 3.03 Hz, 1H), 6.98 (d, J=9.09 Hz, 1H), 7.18 (d, J=3.03 Hz, 1H), 7.39 (d, J=5.31 Hz, 2H), 8.59-8.76 (m, 2H), 11.14 (br. s., 1H). [M+H] calc'd for C₂₀H₂₃N₃O₄ 370; found, 370.

### Compound 120: (S)-3-methoxy-5-(3-(1-methoxypropan-2-yloxy)-5-(4-(methylsulfonyl)phenoxy)phenyl)-1H-pyrazole

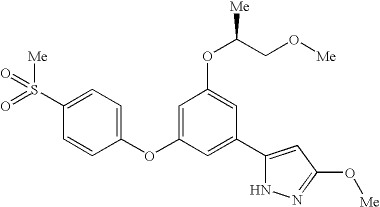

ADDP (0.13 g) was added to a mixture of Compound 114 (0.09 g), (R)-1-methoxypropan-2-ol (0.023 g), tributylphosphine (0.10 g), and 1,4-dioxane (10 mL) at 70° C. The whole was stirred at 70° C. for 2 h, then tributylphosphine (0.10 g) and ADDP (0.13 g) were added to the mixture. The whore was stirred at 70° C. for further 4 h. The whole was concentrated in vacuo. The residue was purified by chromatographed on SiO₂ with hexane-ethyl acetate (2/1 to 1/3, v/v) then by HPLC to give the title compound as a colorless oil (0.03 g). ¹H NMR (400 MHz, CHLOROFORM-d) δ 1.34 (d, J=6.32 Hz, 3H), 3.09 (s, 3H), 3.41-3.44 (m, 3H), 3.48-3.64 (m, 2H), 3.94 (s, 3H), 4.52-4.65 (m, 1H), 5.95 (br. s., 1H), 6.65 (t, J=2.15 Hz, 1H), 6.86 (t, J=1.77 Hz, 1H), 6.99-7.03 (m, 1H), 7.10-7.19 (m, 2H), 7.88-7.96 (m, 2H). [M+H] calc'd for C₂₁H₂₄N₂O₆S 433; found, 433.

### Compound 121: (S)-3-methoxy-5-(3-(1-methoxypropan-2-yloxy)-5-(4-(methylsulfonyl)benzyloxy)phenyl)-1H-pyrazole

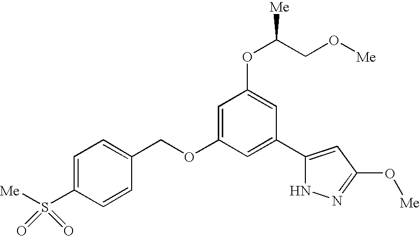

ADDP (0.11 g) was added to a mixture of Compound 115 (0.08 g), (4-(methylsulfonyl)phenyl)methanol (0.055 g), tributylphosphine (0.09 g), THF (2 mL) and toluene (10 mL) at 70° C. The whole was stirred at 70° C. for 15 h, and then concentrated in vacuo. Isopropyl ether was added to the mixture and the insoluble materials were removed by filtration. The filtrate was concentrated in vacuo. The residue was purified by chromatographed on SiO₂ with hexane-ethyl acetate (1/2 to 1/4, v/v) then by HPLC to give the title compound as a colorless oil (0.03 g). ¹H NMR (400 MHz, CHLOROFORM-d) δ 1.34 (d, J=6.06 Hz, 3H), 3.09 (s, 3H), 3.43 (s, 3H), 3.48-3.66 (m, 2H), 3.95 (s, 3H), 4.53-4.65 (m, 1H), 5.19 (s, 2H), 5.95 (br. s., 1H), 6.58 (t, J=2.15 Hz, 1H), 6.76-6.78 (m, 2H), 7.61-7.68 (m, 2H), 7.97-8.02 (m, 2H). [M+H] calc'd for C₂₂H₂₆N₂O₆S 447; found, 447.

### Compound 122: (S)-3-methoxy-5-(3-(1-methoxypropan-2-yloxy)-5-(2-(thiophen-3-yl)ethoxy)phenyl)-1H-pyrazole

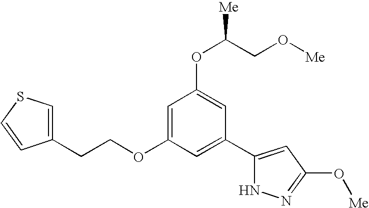

ADDP (0.10 g) was added to a mixture of Compound 115 (0.08 g), 2-(thiophen-3-yl)ethanol (0.045 g), tributylphosphine (0.08 g), THF (2 mL), THF (2 mL) and toluene (10 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was purified by chromatographed on SiO₂ with hexane-ethyl acetate (2/1 to 1/4, v/v) then by HPLC to give the title compound as a colorless oil (0.025 g). ¹H NMR (400 MHz, CHLOROFORM-d) δ 1.34 (d, J=6.32 Hz, 3H), 3.15 (t, J=6.69 Hz, 2H), 3.43 (s, 3H), 3.48-3.63 (m, 2H), 3.95 (s, 3H), 4.20 (t, J=6.82 Hz, 2H), 4.54-4.64 (m, 1H), 5.94 (br. s., 1H), 6.52 (t, J=2.27 Hz, 1H), 6.68 (s, 1H), 6.73 (d, J=1.52 Hz, 1H), 7.06 (dd, J=4.80, 1.26 Hz, 1H), 7.10-7.13 (m, 1H), 7.31 (dd, J=4.93, 2.91 Hz, 1H). [M+H] calc'd for C₂₀H₂₄N₂O₄S 389; found, 389.

### Compound 123: (S)-2-(5-(3-(1-methoxypropan-2-yloxy)-5-(4-(methylsulfonyl)phenoxy)phenyl)-1H-pyrazol-3-yloxy)acetic acid

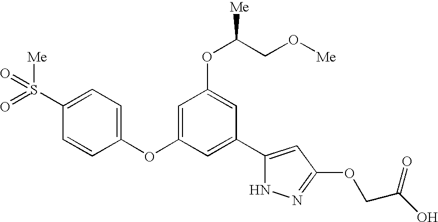

A mixture of Compound 112 (0.27 g), 1N NaOH (2 mL), THF (2 mL), and methanol (2 mL) was stirred at 50° C. for 3 h. 1N HCl (2 mL) and water were added to the mixture and the mixture was extracted with ethyl acetate. The organic layer was washed with brine, dried over MgSO₄ and concentrated in vacuo to give the title compound as a colorless oil. ¹H NMR (400 MHz, CHLOROFORM-d) δ 1.32 (d, J=6.32 Hz, 3H), 3.08 (s, 3H), 3.42 (s, 3H), 3.48-3.64 (m, 2H), 4.53-4.62 (m, 1H), 4.80 (s, 2H), 6.01 (s, 1H), 6.64 (t, J=2.15 Hz, 1H), 6.81 (s, 1H), 6.97 (s, 1H), 7.07-7.16 (m, 2H), 7.88-7.97 (m, 2H). [M+H] calc'd for C₂₂H₂₄N₂O₈S 477; found, 477.

### Compound 124: (S)-1-(4-acetylpiperazin-1-yl)-2-(5-(3-(1-methoxypropan-2-yloxy)-5-(4-(methylsulfonyl)phenoxy)phenyl)-1H-pyrazol-3-yloxy)ethanone

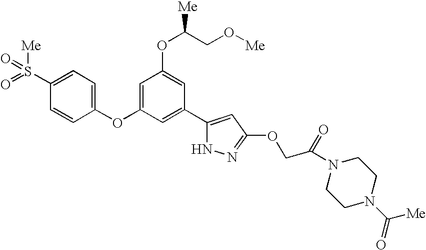

A mixture of Compound 123 (60 mg), 1-(piperazin-1-yl)ethanone (20 mg), 1-(3-dimethylaminopropyl)-3-ethylcarbodiimide hydrochloride (33 mg), N-hydroxybenzotriazole (23 mg), and DMF (5 mL) was stirred at room temperature for 15 h. Water was added to the mixture, and the mixture was extracted with ethyl acetate. The organic layer was washed successively with 1N HCl, sat. NaHCO₃ aq., and brine, dried over MgSO₄, and concentrated in vacuo. The residue was purified by HPLC to give the title compound as a colorless amorphous solid (50 mg). ¹H NMR (400 MHz, CHLOROFORM-d) δ 1.34 (d, J=6.32 Hz, 3H), 2.14 (s, 3H), 3.09 (s, 3H), 3.42 (s, 3H), 3.46-3.74 (m, 10H), 4.52-4.63 (m, 1H), 4.92 (br. s., 2H), 6.02 (br. s., 1H), 6.65 (s, 1H), 6.83 (s, 1H), 6.97 (s, 1H), 7.10-7.21 (m, 2H), 7.85-7.96 (m, 2H). [M+H] calc'd for C₂₈H₃₄N₄O₈S 587; found, 587.

### Compound 125: (S)-2-(5-(3-(1-methoxypropan-2-yloxy)-5-(4-(methylsulfonyl)phenoxy)phenyl)-1H-pyrazol-3-yloxy)-1-morpholinoethanone

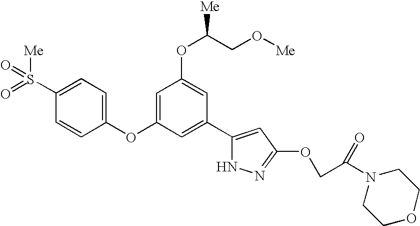

A mixture of Compound 123 (60 mg), morpholine (14 mg), 1-(3-dimethylaminopropyl)-3-ethylcarbodiimide hydrochloride (33 mg), N-hydroxybenzotriazole (23 mg), and DMF (5 mL) was stirred at room temperature for 2.5 days. The mixture was concentrated in vacuo. The residue was purified by HPLC to give the title compound as a colorless oil (40 mg). ¹H NMR (400 MHz, CHLOROFORM-d) δ 1.33 (d, J=6.32 Hz, 3H), 3.08 (s, 3H), 3.42 (s, 3H), 3.48-3.78 (m, 10H), 4.52-4.63 (m, 1H), 4.88 (s, 2H), 5.99 (s, 1H), 6.63 (s, 1H), 6.82 (s, 1H), 6.96 (s, 1H), 7.09-7.17 (m, 2H), 7.85-7.96 (m, 2H). [M+H] calc'd for C₂₆H₃₁N₃O₈S 546; found, 546.

### Compound 126: (S)-N-ethyl-2-(5-(3-(1-methoxypropan-2-yloxy)-5-(4-(methylsulfonyl)phenoxy)phenyl)-1H-pyrazol-3-yloxy)acetamide

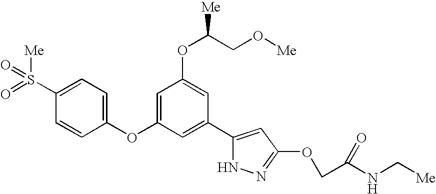

A mixture of Compound 123 (60 mg), ethylamine hydrochloride (16 mg), 1-(3-dimethylaminopropyl)-3-ethylcarbodiimide hydrochloride (33 mg), N-hydroxybenzotriazole (23 mg), triethylamine (20 mg), and DMF (5 mL) was stirred at room temperature for 2.5 days. The mixture was concentrated in vacuo. The residue was purified by HPLC to give the title compound as a colorless oil (40 mg). ¹H NMR (400 MHz, CHLOROFORM-d) δ 1.20 (t, J=7.20 Hz, 3H), 1.35 (d, J=6.32 Hz, 3H), 3.09 (s, 3H), 3.34-3.48 (m, 5H), 3.48-3.66 (m, 2H), 4.54-4.65 (m, 1H), 4.70 (s, 2H), 6.01 (br. s., 1H), 6.57 (br. s., 1H), 6.67 (t, J=2.02 Hz, 1H), 6.83 (s, 1H), 6.99 (s, 1H), 7.09-7.21 (m, 2H), 7.88-7.98 (m, 2H). [M+H] calc'd for C₂₄H₂₉N₃O₇S 504; found, 504.

### Compound 130: 1-isobutyl-6-(1H-pyrazol-5-yl)-1H-benzo[d]imidazole

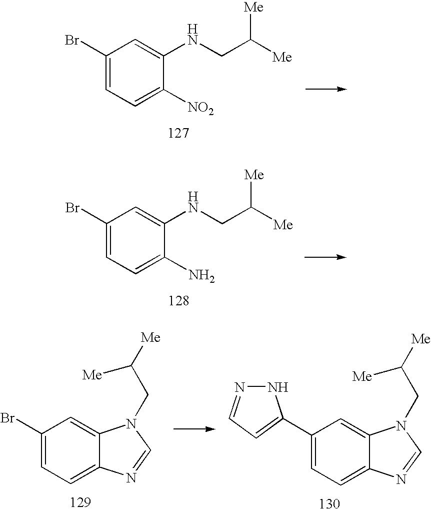

A mixture of 4-bromo-2-fluoro-1-nitrobenzene (1.0 g), triethylamine (0.5 mL), 2-methylpropan-1-amine (0.5 mL) and DMA (5 mL) was stirred at 80° C. for 30 h. Water was added to the mixture and the mixture was extracted with ethyl acetate. The organic layer was washed with brine, dried over MgSO₄, and concentrated in vacuo. The residue was chromatographed on SiO₂ with hexane-ethyl acetate (100/0 to 4/1, v/v) to give 5-bromo-N-isobutyl-2-nitroaniline (compound 127; 1.16 g) as yellow crystals. ¹H NMR (400 MHz, CHLOROFORM-d) δ 1.08 (d, J=6.82 Hz, 6H), 1.92-2.12 (m, 1H), 3.04-3.15 (m, 2H), 6.76 (dd, J=9.09, 2.02 Hz, 1H), 7.03 (d, J=1.77 Hz, 1H), 8.05 (d, J=9.09 Hz, 1H), 8.18 (br. s., 1H).

5-Bromo-N-1-isobutylbenzene-1,2-diamine (compound 128) was synthesized using an analogous procedure to that described in connection with Compound 132 except that Compound 127 was used. Compound 128 was obtained as a brown oil. ¹H NMR (400 MHz, CHLOROFORM-d) δ 1.03-1.05 (m, 6H), 1.87-2.01 (m, 1H), 2.91 (d, J=6.57 Hz, 2H), 3.13-3.52 (m, 3H), 6.55-6.62 (m, 1H), 6.72-6.80 (m, 2H).

A mixture of compound 128 (930 mg) and 99% formic acid (10 mL) was refluxed for 15 h. The mixture was concentrated in vacuo. The residue was diluted with sat.NaHCO₃ aq and the resultant was extracted with ethyl acetate. The organic layer was washed with brine, dried over MgSO₄, and concentrated in vacuo. The residue was chromatographed on SiO₂ with hexane-ethyl acetate (2/1 to 1/9, v/v) to give 6-bromo-1-isobutyl-1H-benzo[d]imidazole (compound 129; 850 mg) as brown crystals. ¹H NMR (400 MHz, CHLOROFORM-d) δ 0.98 (d, J=6.57 Hz, 6H), 2.14-2.34 (m, 1H), 3.95 (d, J=7.33 Hz, 2H), 7.40 (dd, J=8.59, 1.77 Hz, 1H), 7.56 (d, J=1.77 Hz, 1H), 7.69 (d, J=8.59 Hz, 1H), 7.86 (s, 1H).

A mixture of compound 129 (90 mg), 1H-pyrazol-5-ylboronic acid (60 mg), tetrakis(triphenylphosphine)palladium(0) (42 mg), 2M Na₂CO₃ (0.1 mL), ethanol (0.2 mL) and toluene (0.8 mL) was irradiated with microwaves (170° C., 20 min). Water was added to the mixture and the mixture was extracted with ethyl acetate. The organic layer was washed with brine, dried over MgSO₄, and concentrated in vacuo. The residue was purified with HPLC to give the title compound (10 mg) as a colorless oil. ¹H NMR (400 MHz, CHLOROFORM-d) δ 0.99 (d, J=6.57 Hz, 6H), 2.16-2.40 (m, 1H), 4.01 (dd, J=7.20, 5.94 Hz, 2H), 6.73 (br. s., 1H), 7.29-7.48 (m, 3H), 7.70 (d, J=7.33 Hz, 1H), 7.79-7.93 (m, 2H). [M+H] calc'd for C₁₄H₁₆N₄ 241; found, 241.

### Compound 134: 1-(cyclohexylmethyl)-6-(1H-pyrazol-5-yl)-1H-benzo[d]imidazole

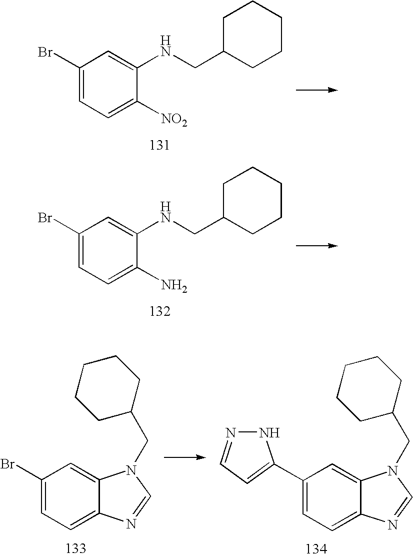

5-Bromo-N-(cyclohexylmethyl)-2-nitroaniline (compound 131) was synthesized using an analogous procedure to that described in connection with Compound 127 except that cyclohexylmethanamine was used. ¹H NMR (400 MHz, CHLOROFORM-d) δ 0.94-1.40 (m, 5H), 1.63-1.95 (m, 6H), 3.06-3.16 (m, 2H), 6.75 (dd, J=9.09, 2.02 Hz, 1H), 7.02 (d, J=1.77 Hz, 1H), 8.04 (d, J=9.09 Hz, 1H), 8.17 (br. s., 1H).

A mixture of compound 131 (1.20 g), iron (powder, 1.06 g), calcium chloride (42 mg) and 80% aqueous ethanol (80 mL) was refluxed for 4 h, then iron (powder, 1.06 g), and calcium chloride (42 mg) were added to the mixture. The whole was refluxed for 15 h. Water was added to the mixture and the mixture was extracted with ethyl acetate. The organic layer was washed with brine, dried over MgSO₄ and concentrated in vacuo. The residue was chromatographed on SiO₂ with hexane-ethyl acetate (10/1 to 2/1, v/v) to give 5-bromo-N1-(cyclohexylmethyl)benzene-1,2-diamine (compound 132) as a brown oil (700 mg). ¹H NMR (400 MHz, CHLOROFORM-d) δ 0.94-1.38 (m, 5H), 1.49-1.97 (m, 6H), 2.93 (d, J=6.82 Hz, 2H), 2.98-3.70 (m, 3H), 6.59 (d, J=7.83 Hz, 1H), 6.67-6.79 (m, 2H)

6-Bromo-1-(cyclohexylmethyl)-1H-benzo[d]imidazole (compound 133) was synthesized using an analogous procedure to that described in connection with Compound 129 except that Compound 132 was used. Compound 133 was obtained as brown crystals. ¹H NMR (400 MHz, CHLOROFORM-d) δ 0.91-1.33 (m, 5H), 1.59-1.96 (m, 6H), 3.97 (d, J=7.07 Hz, 2H), 7.39 (dd, J=8.59, 1.77 Hz, 1H), 7.56 (d, J=1.77 Hz, 1H), 7.68 (d, J=8.59 Hz, 1H), 7.84 (s, 1H).

The title compound was synthesized using an analogous procedure to that described in connection with Compound 130 except that Compound 133 was used. The title compound was obtained as a colorless oil. ¹H NMR (400 MHz, CHLOROFORM-d) δ 0.74-1.24 (m, 5H), 1.51-1.99 (m, 6H), 3.95 (d, J=7.07 Hz, 2H), 6.71 (br. s., 1H), 7.61-7.98 (m, 5H). [M+H] calc'd for C₁₇H₂₀N₄ 281; found, 281.

### Compound 138: 5-(1-isobutyl-1H-benzo[d]imidazol-6-yl)-1H-pyrazol-3-ol

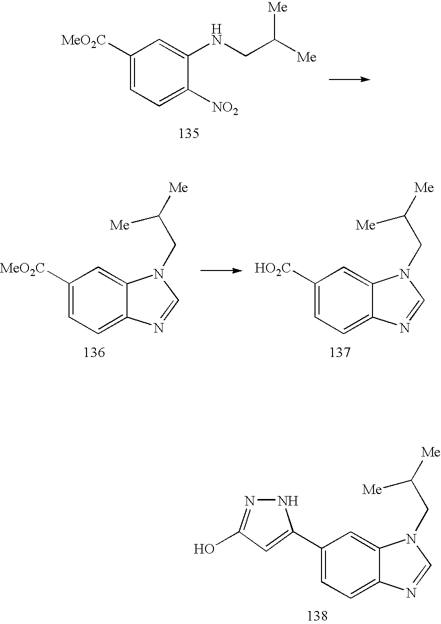

A mixture of methyl 3-fluoro-4-nitrobenzoate (2.0 g), triethylamine (1.55 mL), 2-methylpropan-1-amine (1.1 mL) and DMA (10 mL) was stirred at 80° C. for 3 days. Water was added to the mixture and the mixture was extracted with ethyl acetate. The organic layer was washed with brine, dried over MgSO₄, and concentrated in vacuo. The residue was chromatographed on SiO₂ with hexane-ethyl acetate (9/1 to 4/1, v/v) to give methyl 3-(isobutylamino)-4-nitrobenzoate (compound 135; 1.16 g) as orange crystals. ¹H NMR (400 MHz, CHLOROFORM-d) δ 1.09 (d, J=6.82 Hz, 6H), 1.96-2.12 (m, 1H), 3.17-3.25 (m, 2H), 3.97 (s, 3H), 7.23 (d, J=8.84 Hz, 1H), 7.57 (s, 1H), 8.13 (br. s., 1H), 8.24 (d, J=8.84 Hz, 1H).

Formic acid (99%, 50 mL) was added to a mixture of compound 135 (1.87 g), 10% Pd—C (300 mg) and methanol (10 mL) at room temperature. The whole was refluxed for 15 h. The catalyst was removed by filtration. The filtrate was concentrated in vacuo. The residue was diluted with sat.NaHCO₃ aq and the resultant was extracted with ethyl acetate. The organic layer was washed with brine, dried over MgSO₄, and concentrated in vacuo. The residue was chromatographed on SiO₂ with hexane-ethyl acetate (1/2 to 1/4, v/v) to give methyl 1-isobutyl-1H -benzo[d]imidazole-6-carboxylate (compound 136; 1.65 g) as colorless crystals. ¹H NMR (400 MHz, CHLOROFORM-d) δ 0.99 (d, J=6.57 Hz, 6H), 2.21-2.35 (m, 1H), 3.98 (s, 3H), 4.04 (d, J=7.33 Hz, 2H), 7.83 (d, J=8.59 Hz, 1H), 7.97-8.03 (m, 2H), 8.16 (s, 1H).

A mixture of Compound 136 (1.65 g), 1N NaOH (15 mL), THF (15 mL), and methanol (15 mL) was stirred at 60° C. for 3 h. 1N HCl (15 mL) and water were added to the mixture. The mixture was extracted with ethyl acetate. The organic layer was washed with brine, dried over MgSO₄, and concentrated in vacuo to give 1-isobutyl-1H-benzo[d]imidazole-6-carboxylic acid (compound 137; 1.50 g) as colorless crystals. ¹H NMR (400 MHz, CHLOROFORM-d) δ 1.02 (d, J=6.57 Hz, 6H), 2.22-2.43 (m, 1H), 4.09 (d, J=7.33 Hz, 2H), 7.94 (d, J=8.59 Hz, 1H), 8.05-8.20 (m, 2H), 8.29 (s, 1H).

Compound 138 was synthesized using an analogous procedure to that described in connection with Compound 84 except that Compound 137 was used. Compound 138 was obtained as pale pink crystals. ¹H NMR (400 MHz, DMSO-d) δ 0.88 (d, J=6.57 Hz, 6H), 2.09-2.31 (m, 1H), 4.07 (d, J=7.33 Hz, 2H), 5.96 (br. s., 1H), 7.52 (d, J=8.34 Hz, 1H), 7.65 (d, J=8.34 Hz, 1H), 7.92 (s, 1H), 8.23 (s, 1H), 9.63 (br. s., 1H), 12.03 (br. s., 1H). [M+H] calc'd for C₁₄H₁₆N₄O 257; found, 257.

### Compound 142: 5-(1-(cyclohexylmethyl)-1H-benzo[d]imidazol-6-yl)-1H-pyrazol-3-ol

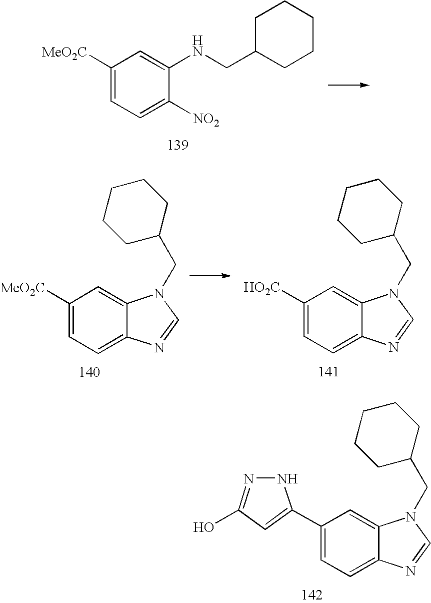

Methyl 3-(cyclohexylmethylamino)-4-nitrobenzoate (compound 139) was synthesized using an analogous procedure to that described in connection with Compound 135 except that cyclohexylmethanamine was used. Compound 139 was obtained as orange crystals. ¹H NMR (400 MHz, CHLOROFORM-d) δ 1.00-1.40 (m, 5H), 1.65-1.94 (m, 6H), 3.20-3.25 (m, 2H), 3.97 (s, 3H), 7.22 (dd, J=8.84, 1.77 Hz, 1H), 7.57 (d, J=1.77 Hz, 1H), 8.13 (br. s., 1H), 8.23 (d, J=8.84 Hz, 1H).

Methyl 1-(cyclohexylmethyl)-1H-benzo [d]imidazole-6-carboxylate (compound 140) was synthesized using an analogous procedure to that described in connection with Compound 136 except that compound 139 was used. Compound 140 was obtained as orange crystals. ¹H NMR (400 MHz, CHLOROFORM-d) δ 1.00-1.40 (m, 5H), 1.65-1.94 (m, 6H), 3.20-3.25 (m, 2H), 3.97 (s, 3H), 7.22 (dd, J=8.84, 1.77 Hz, 1H), 7.57 (d, J=1.77 Hz, 1H), 8.13 (br. s., 1H), 8.23 (d, J=8.84 Hz, 1H).

1-(Cyclohexylmethyl)-1H-benzo[d]imidazole-6-carboxylic acid (compound 141) was synthesized using an analogous procedure to that described in connection with Compound 137 except that compound 140 was used. Compound 141 was obtained as colorless crystals. ¹H NMR (400 MHz, DMSO-d) δ 0.92-1.24 (m, 5H), 1.44-1.90 (m, 6H), 4.18 (d, J=7.33 Hz, 2H), 7.72 (d, J=8.59 Hz, 1H), 7.83 (dd, J=8.34, 1.26 Hz, 1H), 8.22 (s, 1H), 8.41 (s, 1H), 12.86 (br. s., 1H).

Compound 142 was synthesized using an analogous procedure to that described in connection with Compound 84 except that Compound 141 was used. Compound 142 was obtained as colorless crystals. ¹H NMR (400 MHz, DMSO-d) δ 0.89-1.26 (m, 5H), 1.46-1.98 (m, 6H), 4.10 (d, J=7.33 Hz, 2H), 5.96 (br. s., 1H), 7.51 (d, J=8.08 Hz, 1H), 7.65 (d, J=8.34 Hz, 1H), 7.91 (s, 1H), 8.22 (s, 1H), 9.61 (br. s., 1H), 12.04 (br. s., 1H). [M+H] calc'd for C₁₇H₂₀N₄O 297; found, 297.

### Compound 143: 1-isobutyl-6-(3-methoxy-1H-pyrazol-5-yl)-1H-benzo[d]imidazole

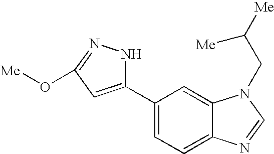

ADDP (0.39 g) was added to a mixture of Compound 138 (0.20 g), methanol (50 mg), tributylphosphine (0.32 g), and 1,4-dioxane (20 mL) at 70° C. The whole was stirred at 70° C. for 15 h, then concentrated in vacuo. Isopropyl ether was added to the mixture and the insoluble materials were removed by filtration. The filtrate was concentrated in vacuo. The residue was purified by chromatography on SiO₂ with hexane-ethyl acetate (1/5 to 1/9, v/v) and then by HPLC to give the title compound as colorless crystals (20 mg). ¹H NMR (400 MHz, DMSO-d₆) δ 0.89 (d, J=6.82 Hz, 6H), 2.12-2.31 (m, 1H), 3.82 (s, 3H), 4.08 (d, J=7.33 Hz, 2H), 6.22 (s, 1H), 7.55 (d, J=8.08 Hz, 1H), 7.69 (d, J=8.34 Hz, 1H), 7.96 (s, 1H), 8.27 (br. s., 1H), 12.33 (br. s., 1H). [M+H] calc'd for C₁₅H₁₈N₄O 271; found, 271.

### Compound 144: 1-(cyclohexylmethyl)-6-(3-methoxy-1H-pyrazol-5-yl)-1H-benzo[d]imidazole

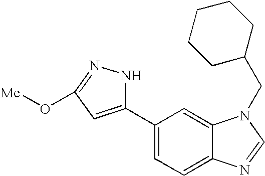

The title compound was synthesized using an analogous procedure to that described in connection with Compound 143 except that Compound 142 was used. The title compound was obtained as colorless crystals. ¹H NMR (400 MHz, DMSO-d₆) δ 0.88-1.32 (m, 5H), 1.44-1.73 (m, 5H), 1.75-1.97 (m, 1H), 3.82 (s, 3H), 4.10 (d, J=7.33 Hz, 2H), 6.22 (s, 1H), 7.54 (d, J=8.34 Hz, 1H), 7.68 (d, J=8.59 Hz, 1H), 7.95 (s, 1H), 8.24 (s, 1H), 12.33 (br. s., 1H). [M+H] calc'd for C₁₈H₂₂N₄O 311; found, 311.

In addition to the foregoing, the above reaction schemes, and variations thereof, have been used to prepare the following:

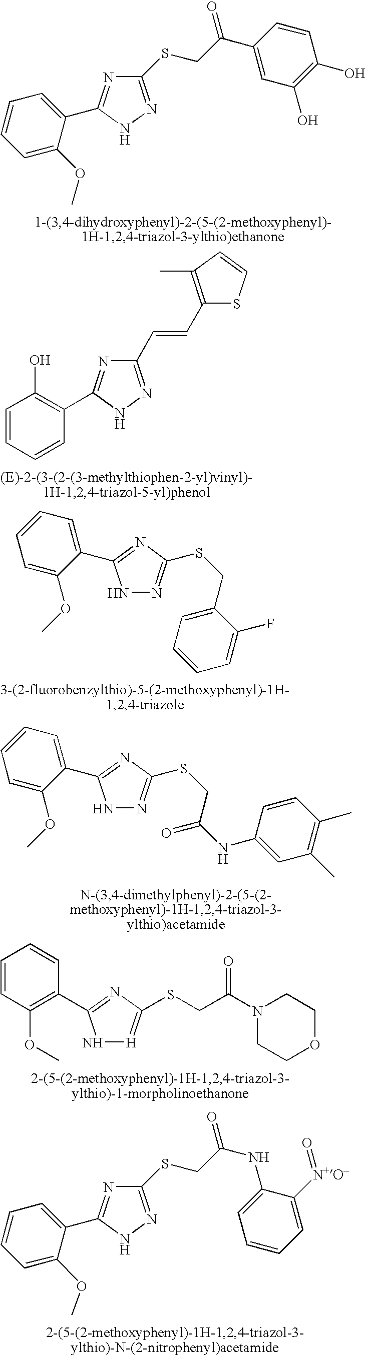

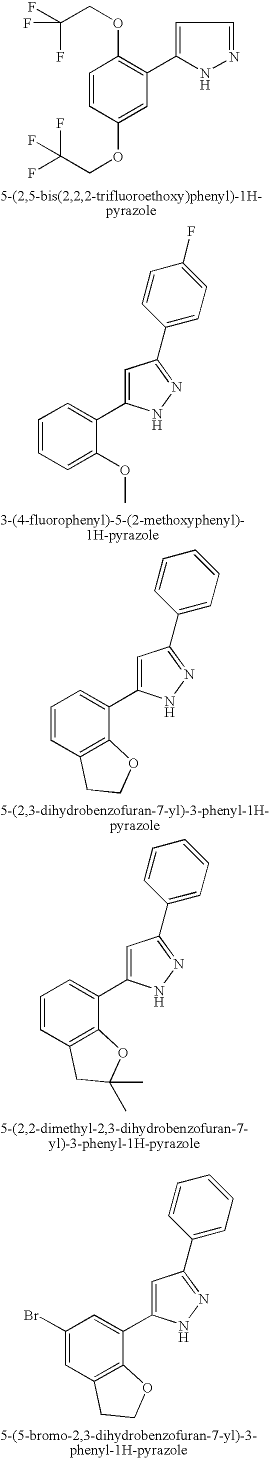

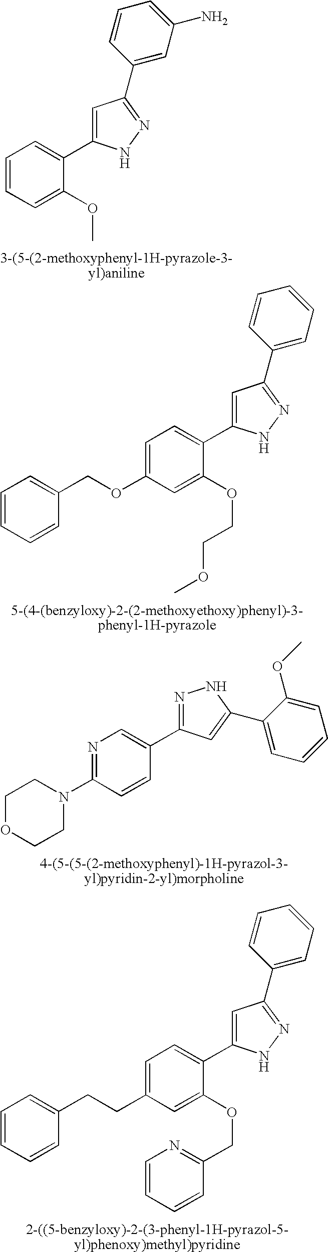

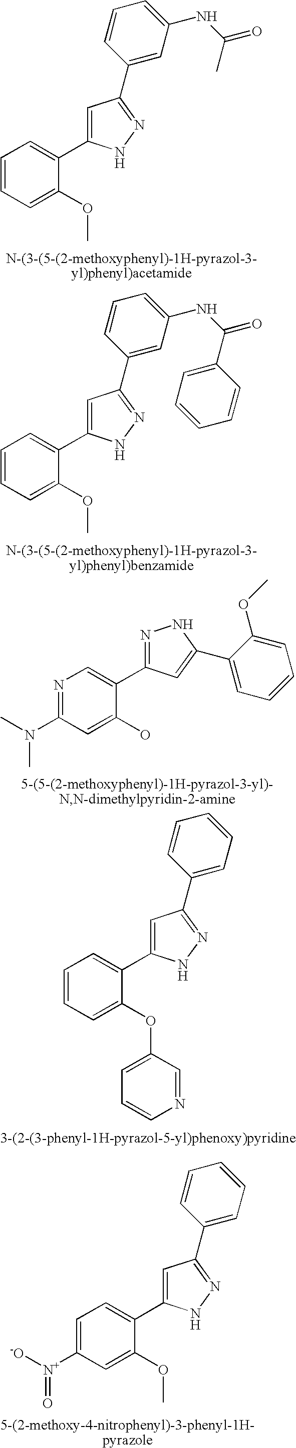

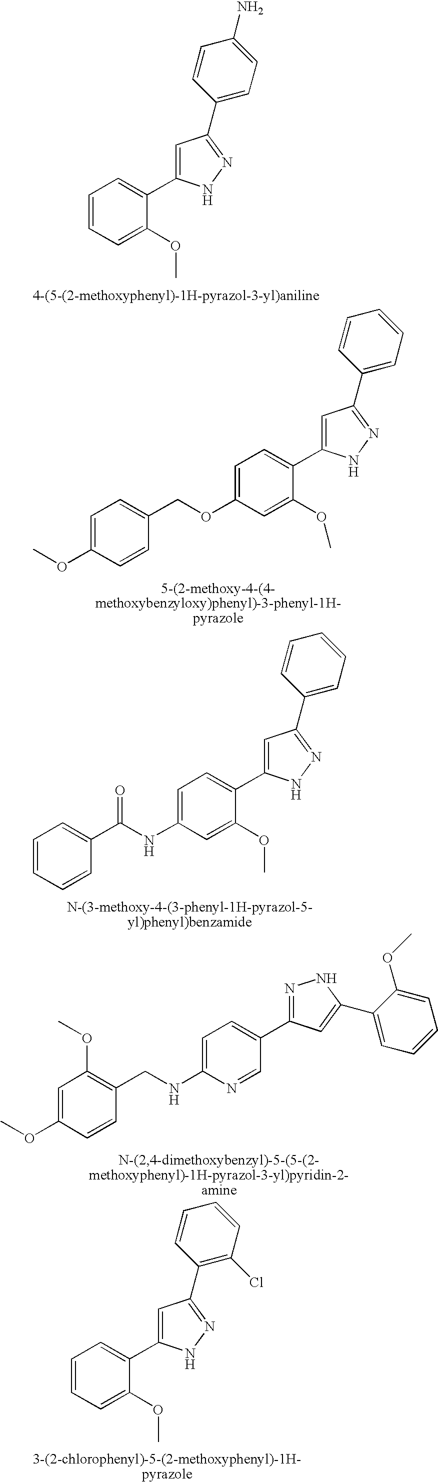

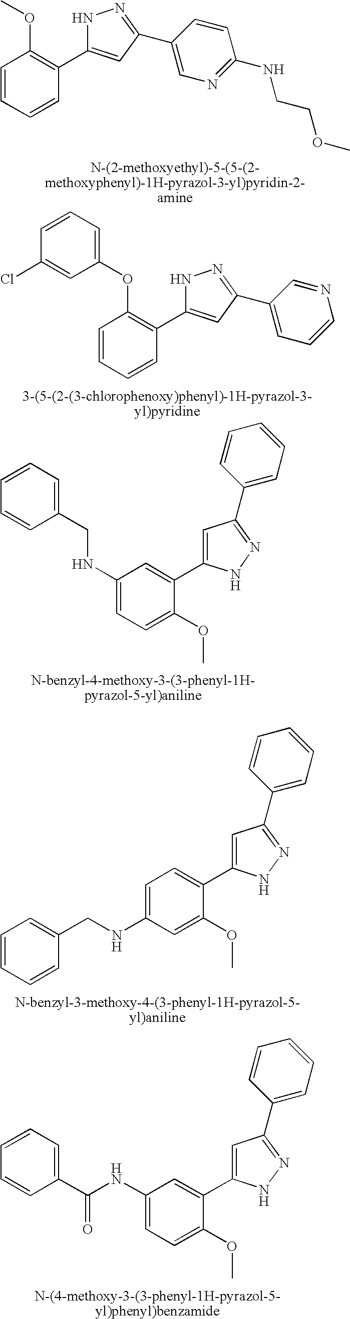

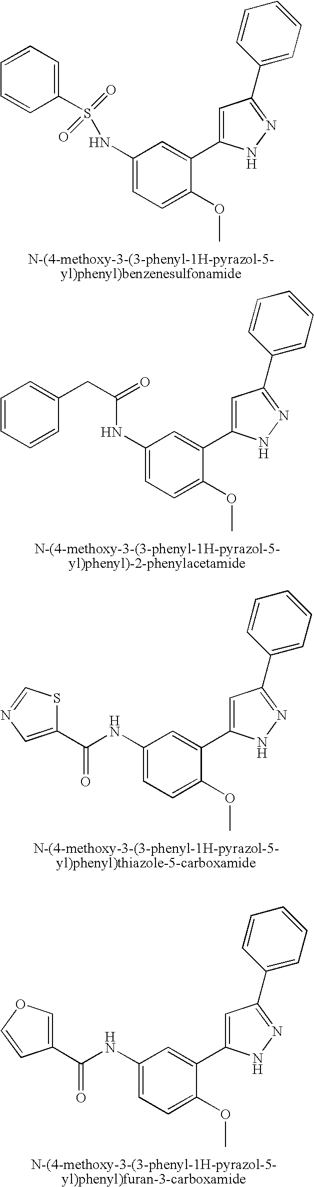

Furthermore, the above reaction schemes, and variations thereof, can be used to prepare the following: 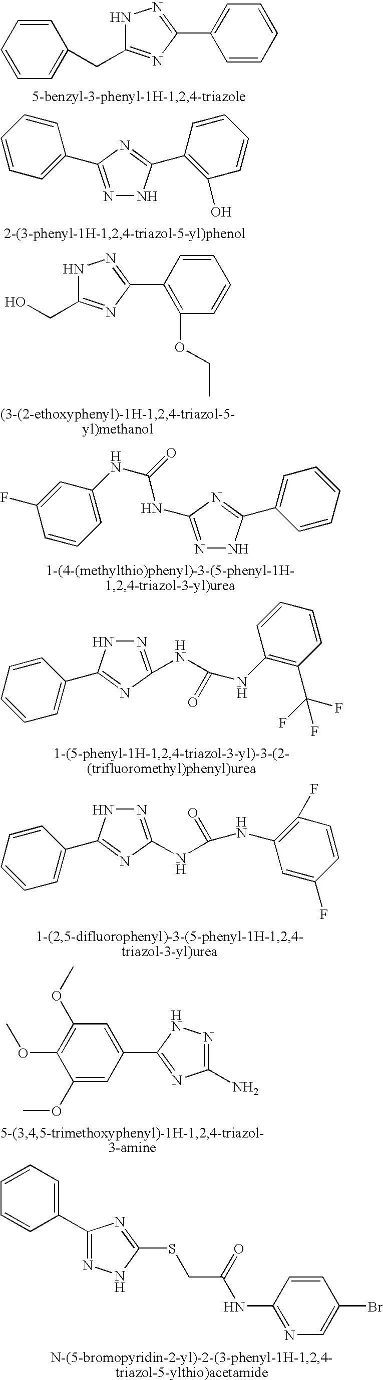 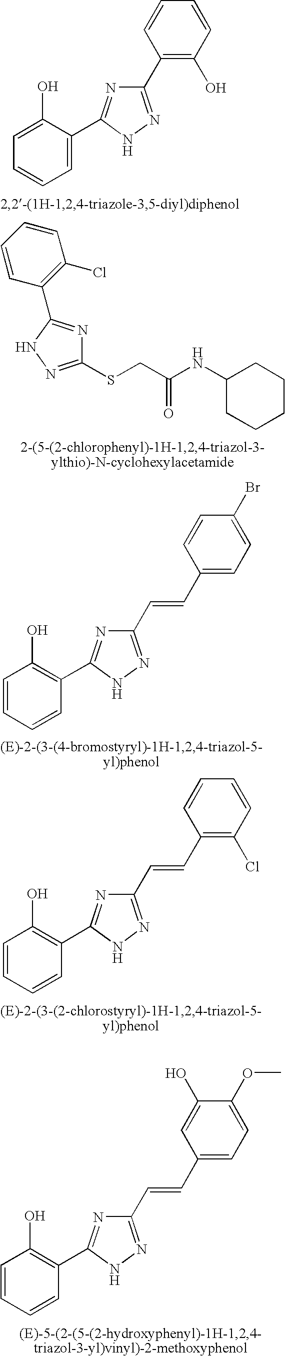 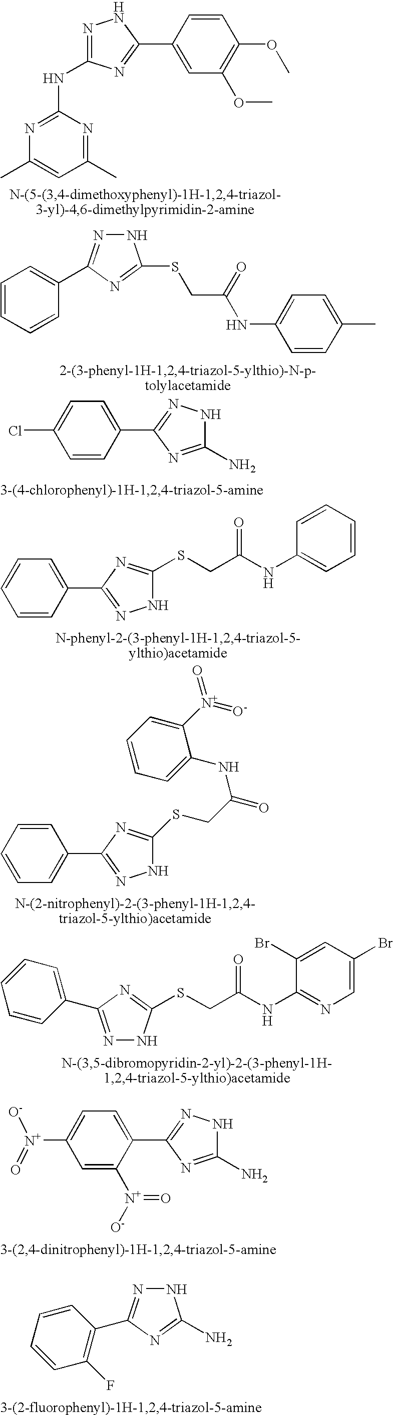 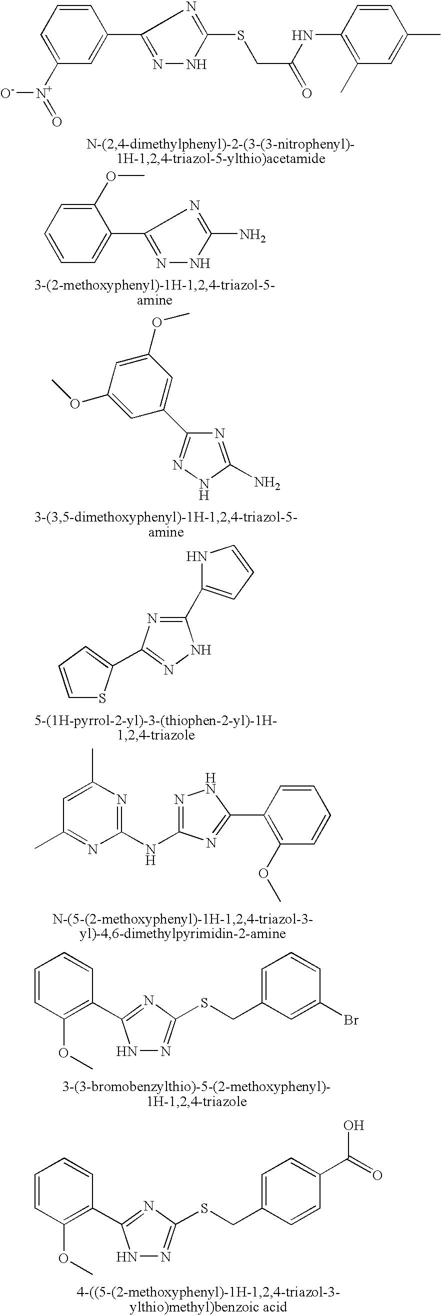 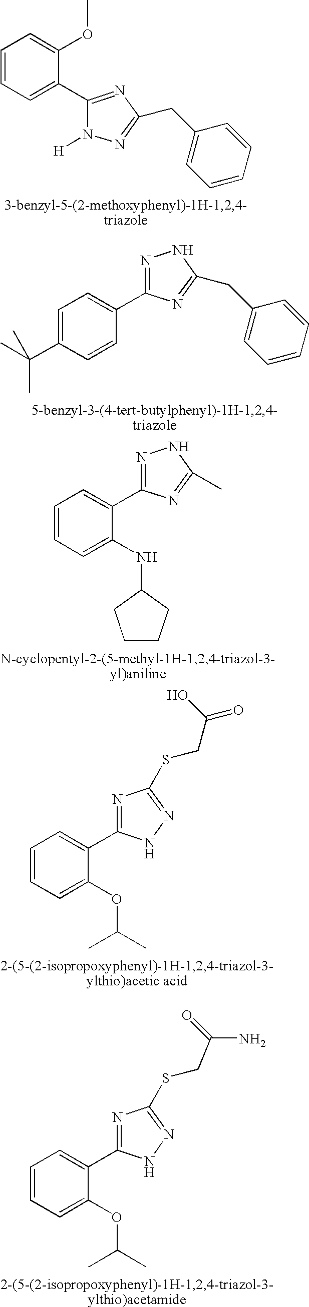 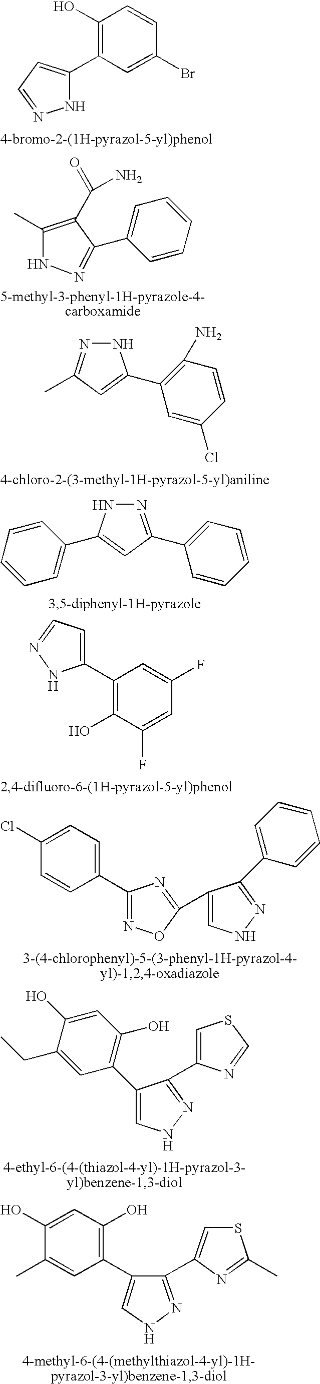 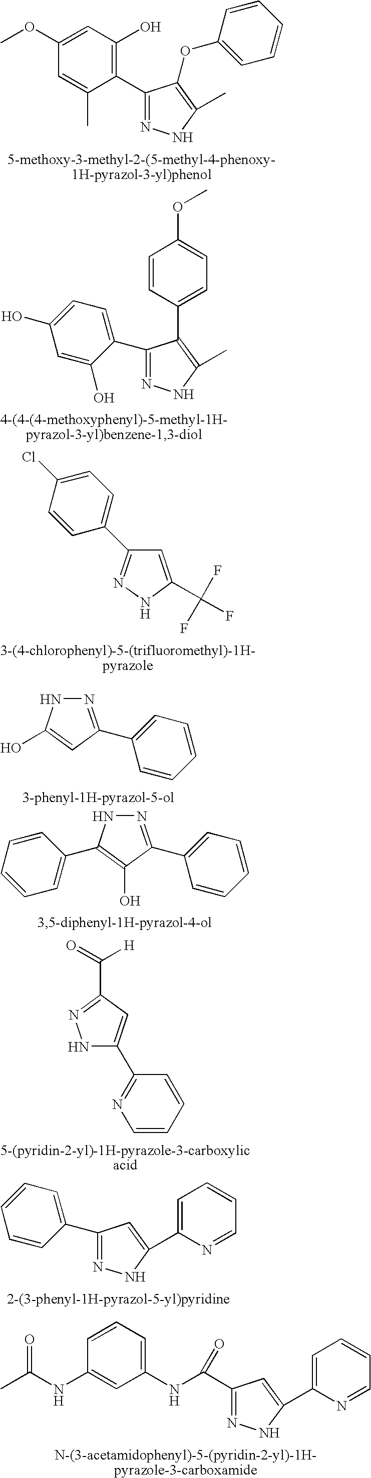 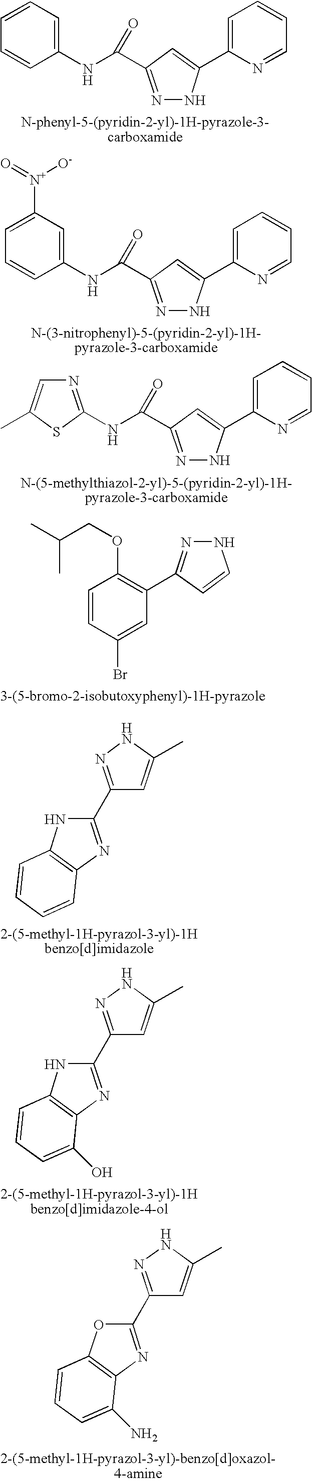 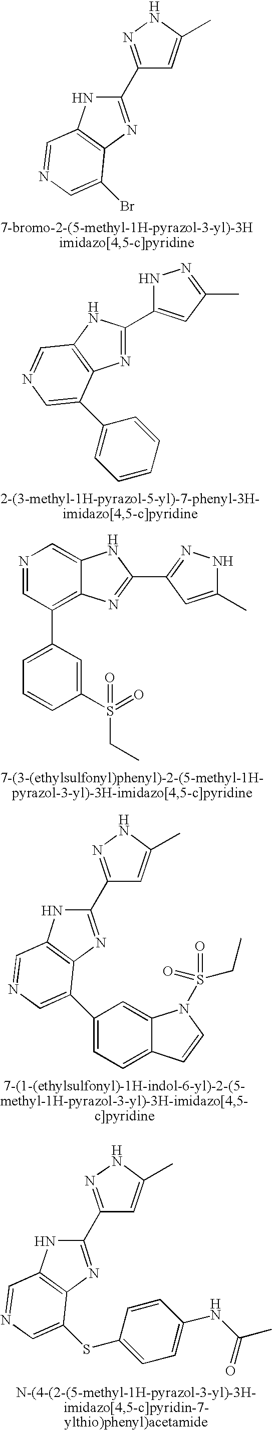 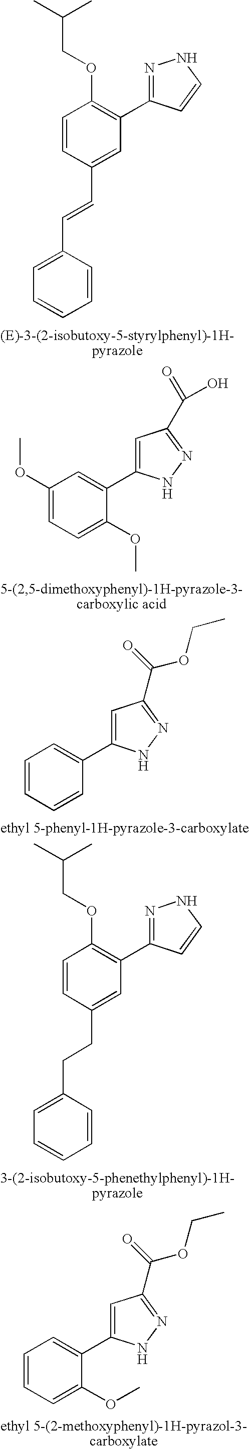 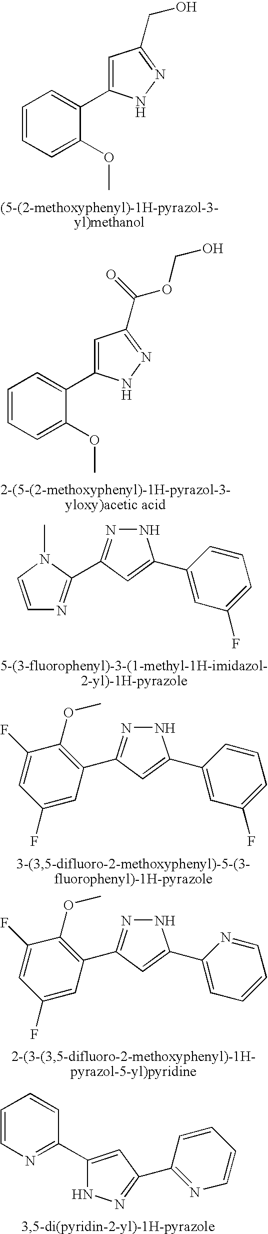 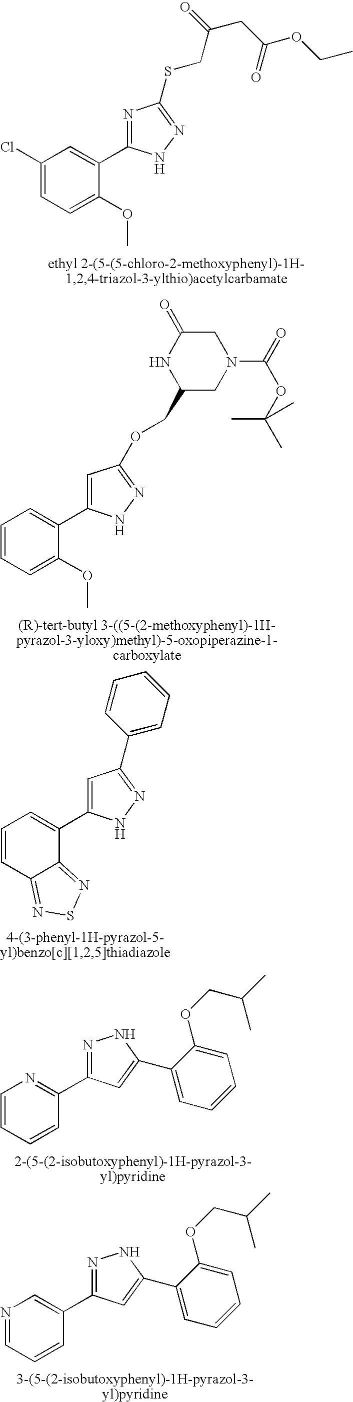 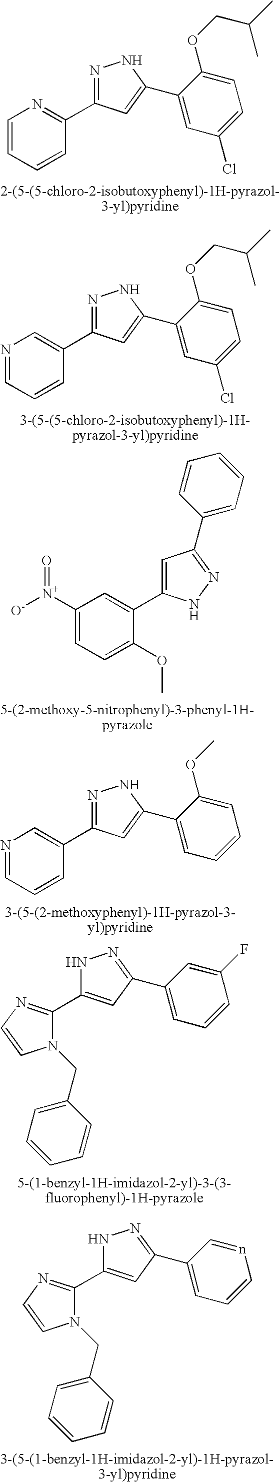 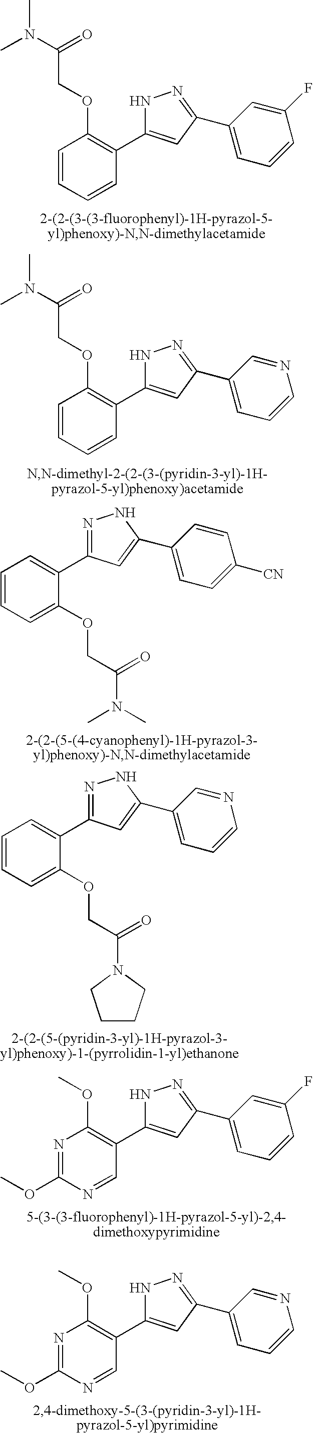 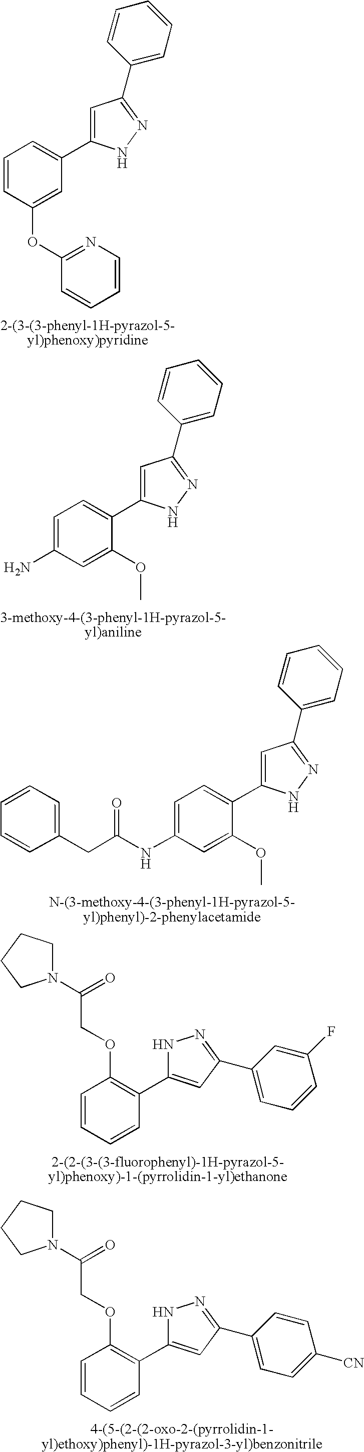 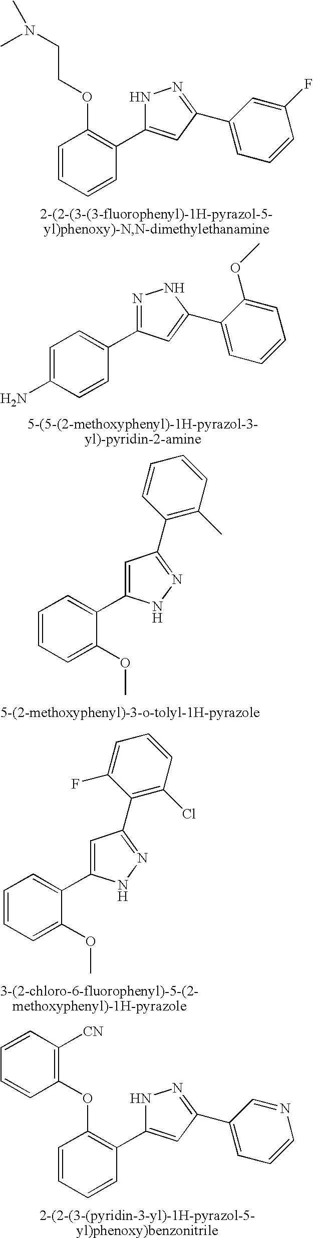 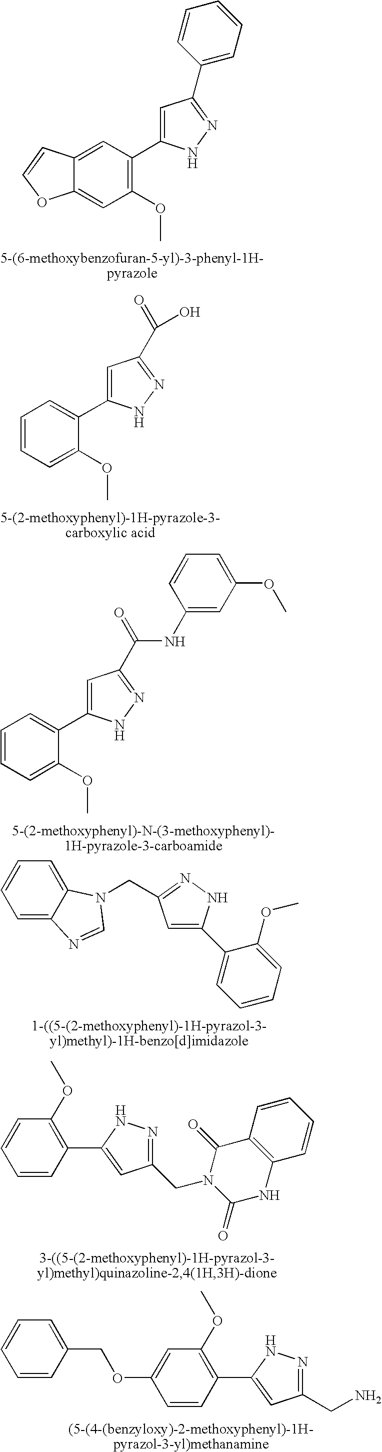 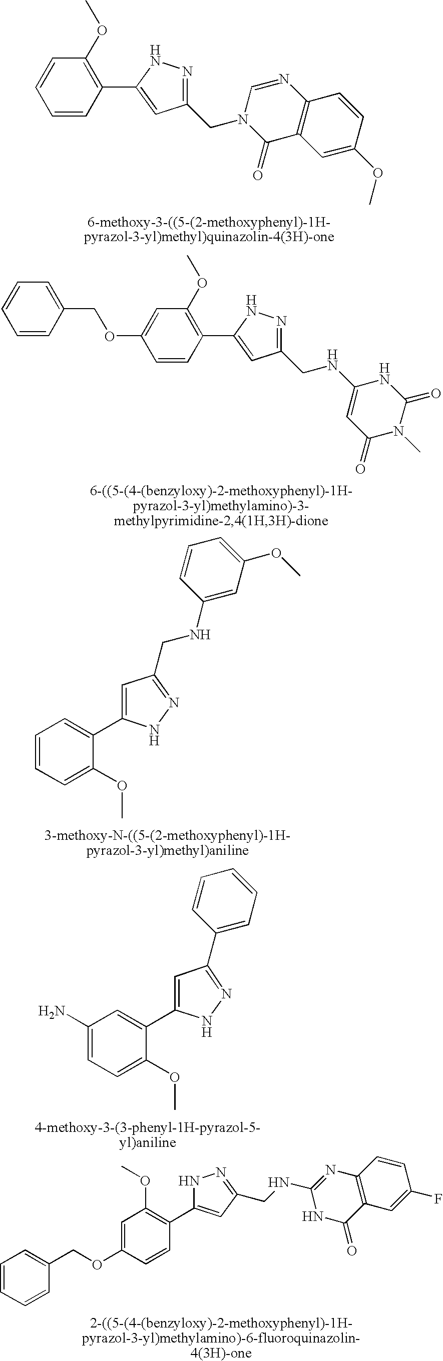 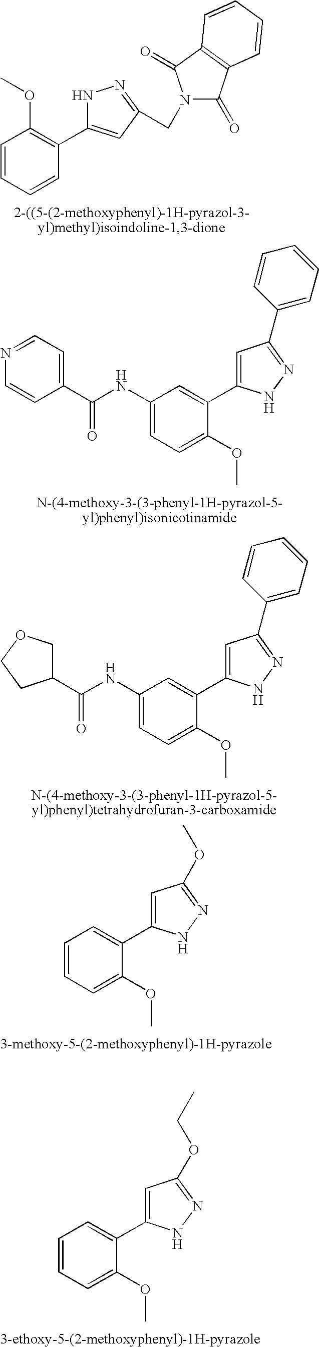 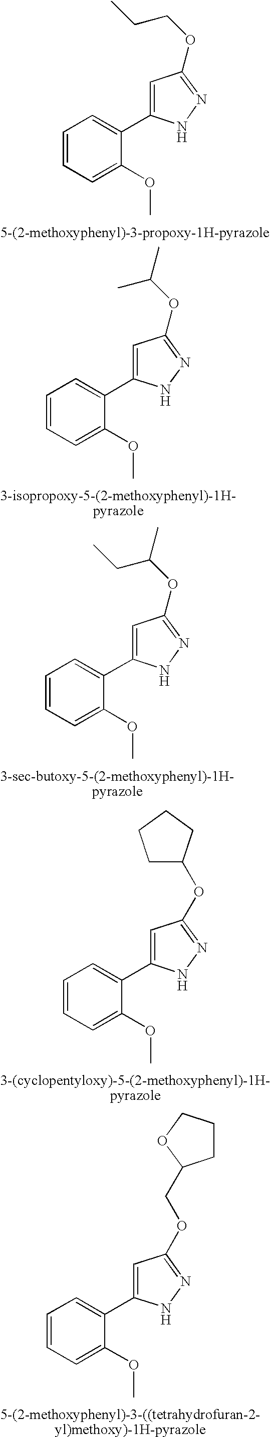 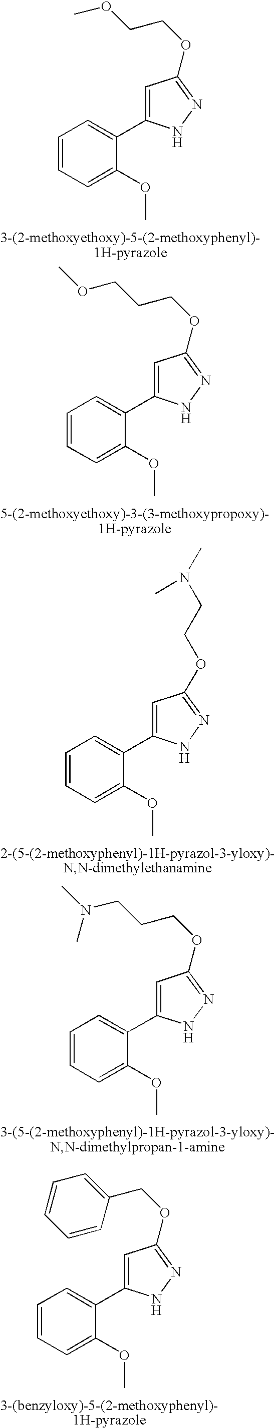 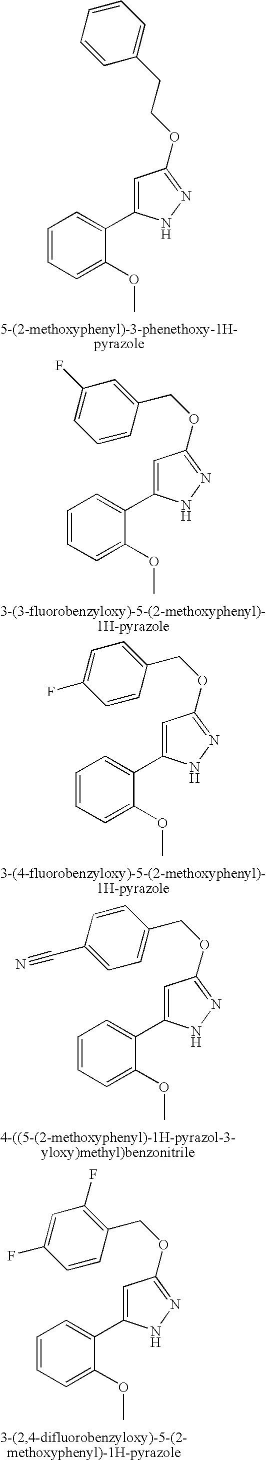 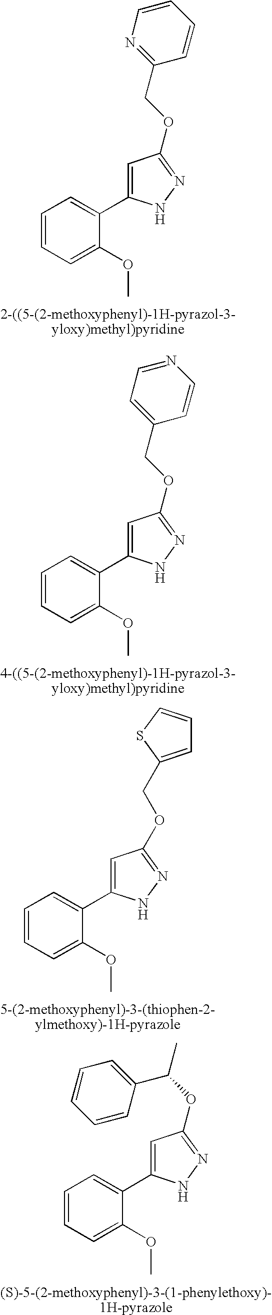 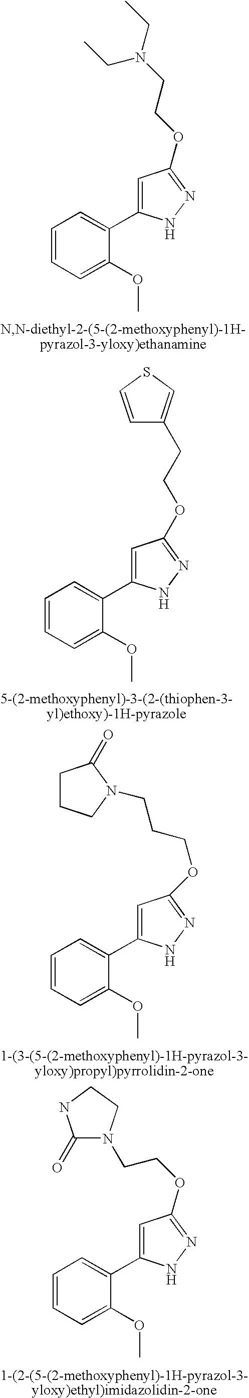 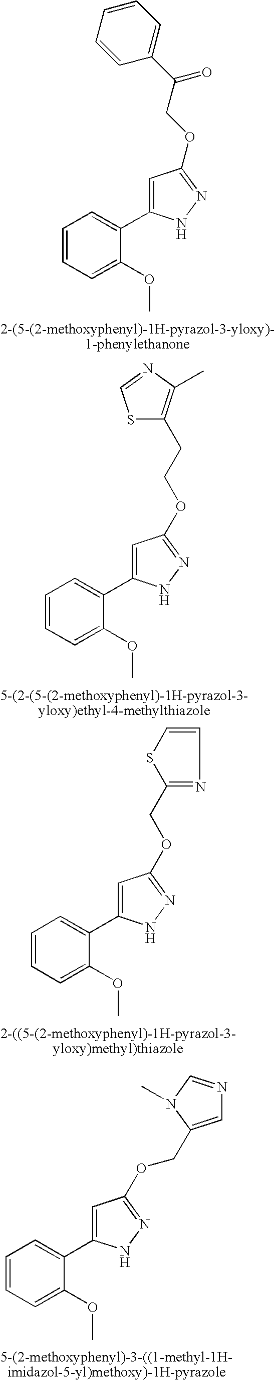 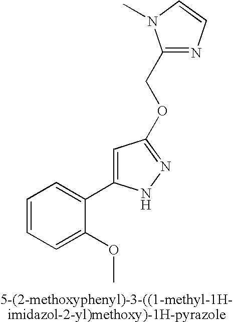
Biological Testing

The activity of compounds as glucokinase activators may be assayed in vitro, in vivo or in a cell line. Provided below is an enzymatic glucokinase activity assay.

Purified glucokinase may be obtained as follows. DNA encoding residues 12-465 of the full-length sequence of the human enzyme may be amplified by PCR and cloned into the HindIII and EcoRI sites of pFLAG-CTC (Sigma). SEQ. I.D. No. 1 corresponds to residues 13-466 of glucokinase.

The expression of recombinant glucokinase protein may be carried out by transformation and growth of DH10b-T1r E. coli cells incorporating the (pFLAG-CTC) plasmid in LB media. Protein expression can be induced in this system by the addition of IPTG to the culture medium.

Recombinant protein may be isolated from cellular extracts by passage over Sepharose Q Fast Flow resin (Pharmacia). This partially purified GK extract may then be further purified by a second passage over Poros HQ10 (Applied Biosystems). The purity of GK may be determined on denaturing SDS-PAGE gel. Purified GK may then be concentrated to a final concentration of 20.0 mg/ml. After flash freezing in liquid nitrogen, the proteins can be stored at −78° C. in a buffer containing 25 mM TRIS-HCl pH 7.6, 50 mM NaCl, and 0.5 mM TCEP.

It should be noted that a variety of other expression systems and hosts are also suitable for the expression of glucokinase, as would be readily appreciated by one of skill in the art.

The activation properties of compounds for GK may be determined using a black 384-well-plate format under the following reaction conditions: 25 mM Hepes pH 7.2, 25 mM NaCl, 10 mM MgCl₂, 0.01% Brij35, 1 mM DTT, 5 μM ATP, 5 mM Glucose 2% DMSO. The amount of ATP consumed may be determined quantitatively by addition of equal volume of luciferase reagent (luciferase+beetle luciferin—KinaseGlo Luminescent Kinase Assay kit from Promega). The luminescence intensity may be measured by using the Analyst HT from LJL Biosystems.

The assay reaction may be initiated as follows: 4 μl of substrate mixture (12.5 μM ATP and 12.5 mM Glucose) was added to each well of the plate, followed by the addition of 2 μl of activator (2 fold serial dilutions for 11 data points for each activator) containing 10% DMSO. 4 μL of 1.25 nM GK solution may be added to initiate the reaction. The reaction mixture may then be incubated at room temperature for 60 min, and quenched and developed by addition of 10 μL of luciferase reagent. Luminescence intensities of the resulting reaction mixtures may be measured after a 10 min incubation at room temperature. The luminescence intensity may be measured by using the Analyst HT from LJL Biosystems.

pK_{act} and % ACTₘₐₓ values may be calculated by non-linear curve fitting of the compound concentrations and luminescence intensities to a standard inhibition/activation equation. K_{act} is the concentration that displays 50% of the maximal increase in GK activity observed using a saturating activator concentration. % Actₘₐₓ represents the calculated maximal gain in GK enzyme activity at a saturating concentration of the compound. pK_{act} and % ACTₘₐₓ values for select compounds of the present invention are given in Table 1.

TABLE 1
pK_{act} and % ACTₘₐₓ of Exemplified Compounds Against GK

| EXAMPLE | pK_{act} |
|---|---|
| 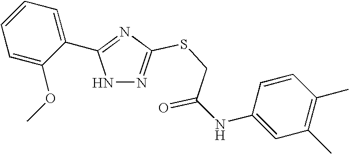 | ≧5.7 |
| 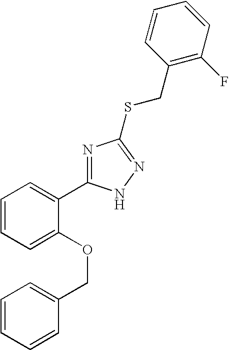 | ≧5.7 |
| 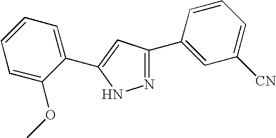 | 5.4-5.6 |
| 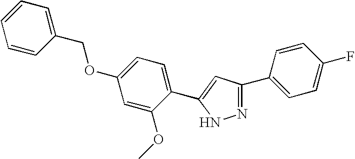 | ≧5.7 |
| 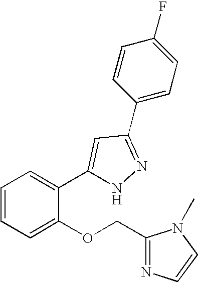 | ≧5.7 |
| 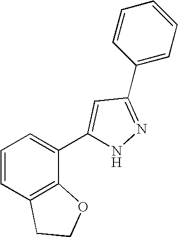 | 5.4-5.6 |
| 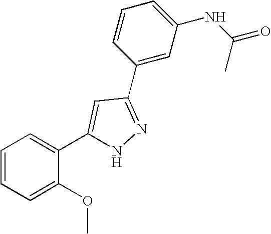 | ≦5.3 |
| 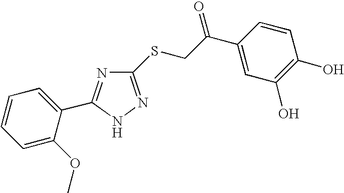 | 5.4-5.6 |
| 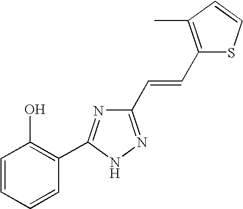 | ≧5.7 |
| 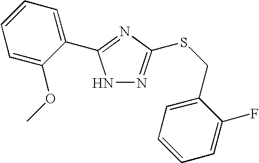 | 5.4-5.6 |
| 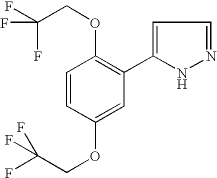 | ≧5.7 |
| 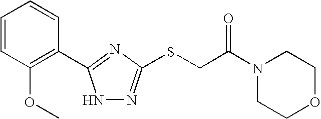 | ≧5.7 |
| 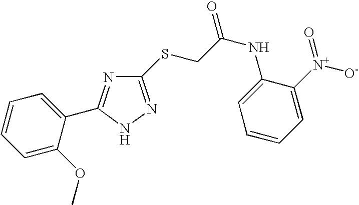 | ≧5.7 |
| 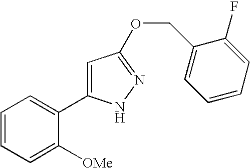 | ≦5.3 |
| 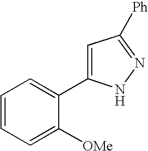 | 5.4-5.6 |
| 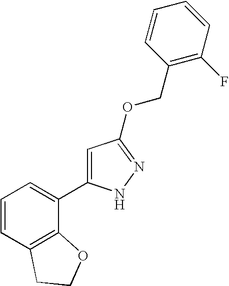 | ≦5.3 |
| 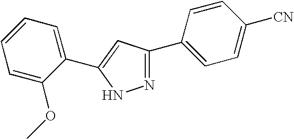 | 5.4-5.6 |
| 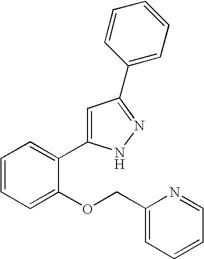 | 5.4-5.6 |
| 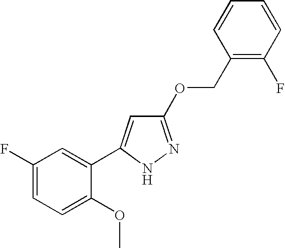 | ≦5.3 |
| 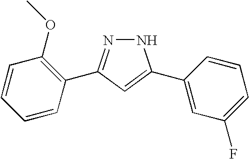 | ≦5.3 |
| 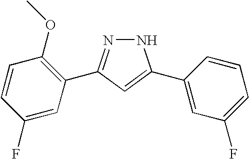 | 5.4-5.6 |
| 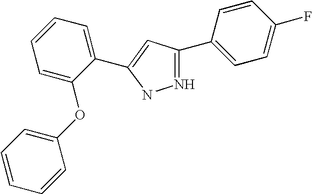 | 5.4-5.6 |
| 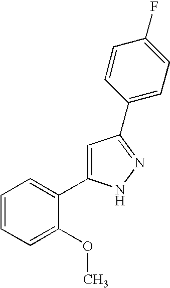 | 5.4-5.6 |
| 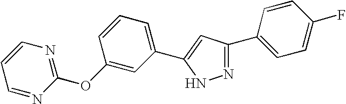 | ≦5.3 |
| 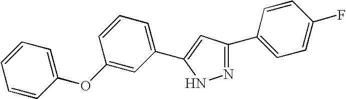 | ≧5.7 |
| 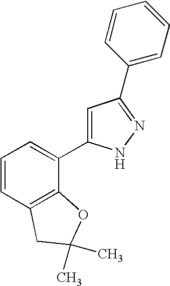 | ≧5.7 |
| 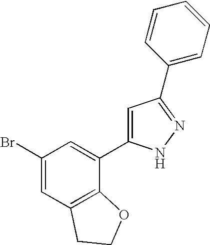 | ≦5.3 |
| 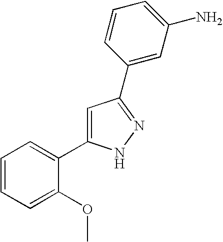 | 5.4-5.6 |
| 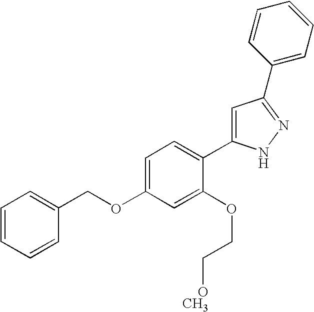 | ≧5.7 |
| 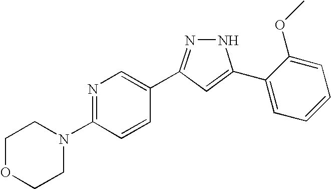 | ≦5.3 |
| 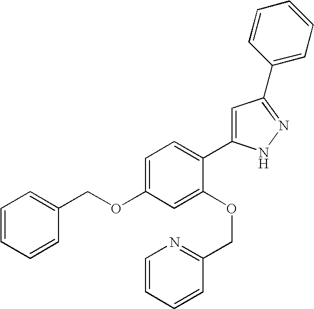 | ≧5.7 |
| 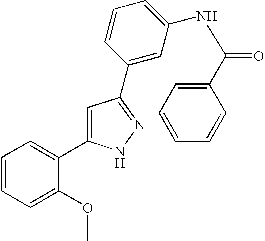 | 5.4-5.6 |
| 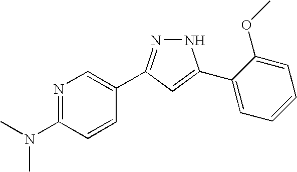 | 5.4-5.6 |
| 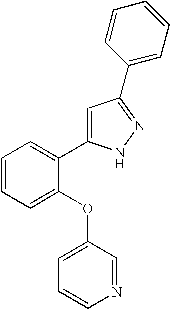 | 5.4-5.6 |
| 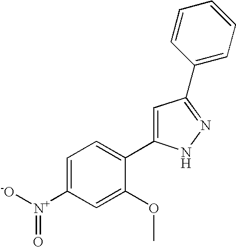 | ≧5.7 |
| 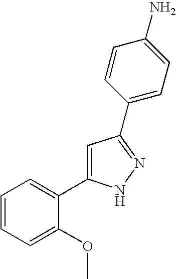 | ≦5.3 |
| 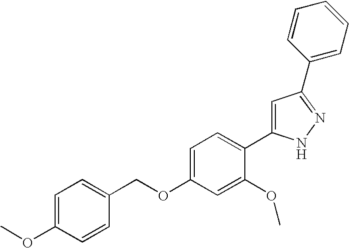 | 5.4-5.6 |
| 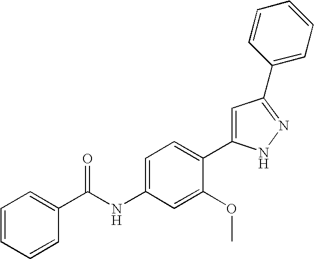 | ≧5.7 |
| 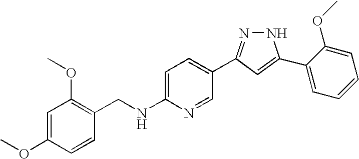 | ≧5.7 |
| 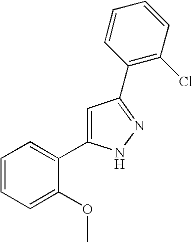 | 5.4-5.6 |
| 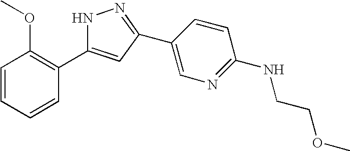 | 5.4-5.6 |
| 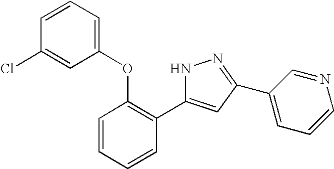 | 5.4-5.6 |
| 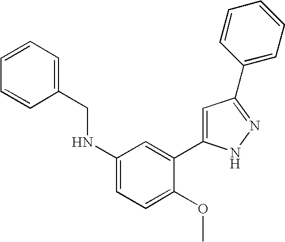 | ≦5.3 |
| 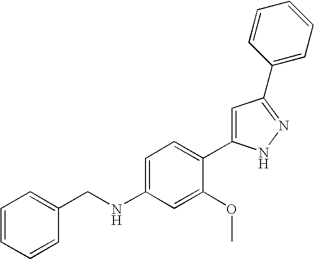 | 5.4-5.6 |
| 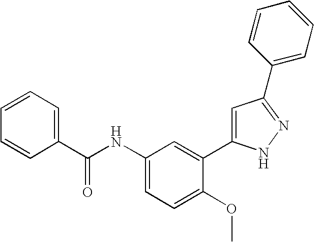 | 5.4-5.6 |
| 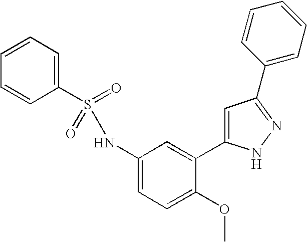 | 5.4-5.6 |
| 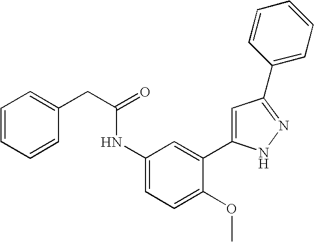 | ≧5.7 |
| 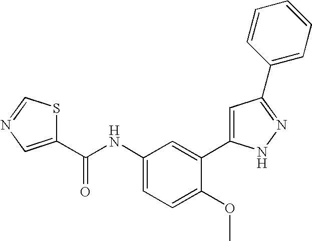 | ≦5.3 |
| 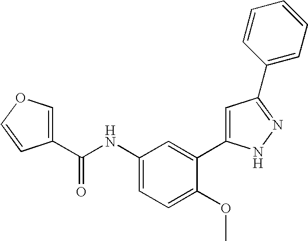 | 5.4-5.6 |
| 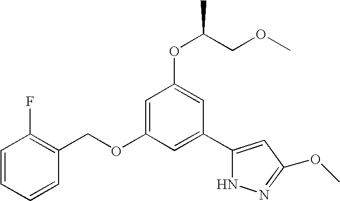 | ≦5.3 |
| 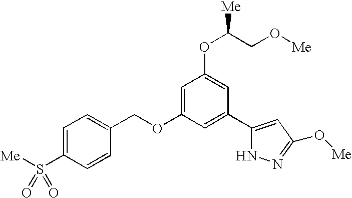 | 5.4-5.6 |
| 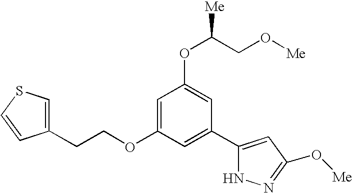 | ≧5.7 |
| 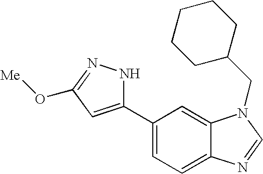 | ≦5.3 |
| 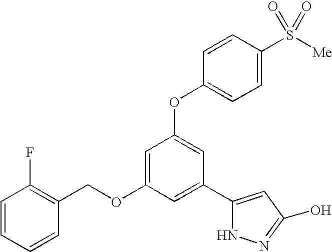 | ≦5.3 |
| 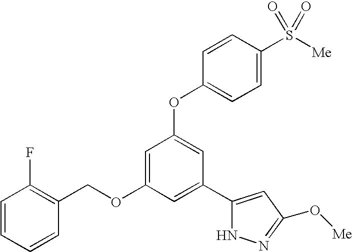 | ≧5.7 |
| 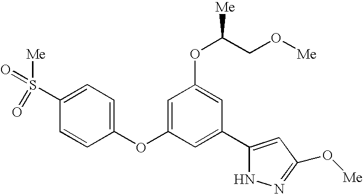 | ≧5.7 |
| 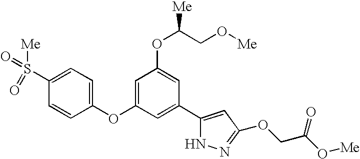 | 5.4-5.6 |
| 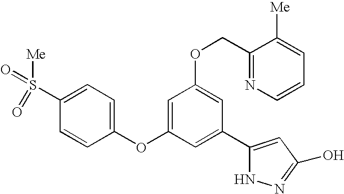 | ≦5.3 |
| 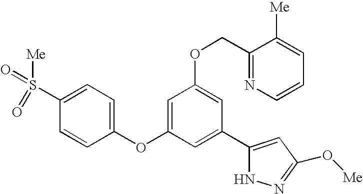 | ≧5.7 |
| 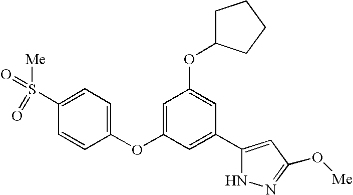 | ≧5.7 |
| 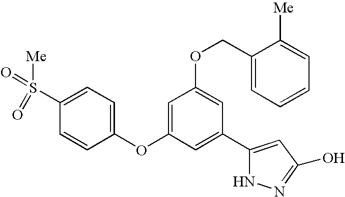 | ≦5.3 |
| 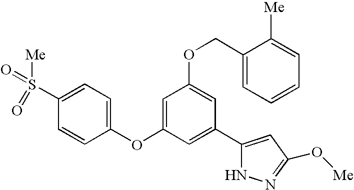 | ≧5.7 |

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A compound comprising: 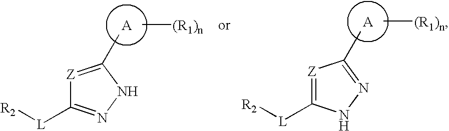
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
Z is selected from the group consisting of CR₃ and N;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

2. The compound of claim 1, wherein the compound comprises

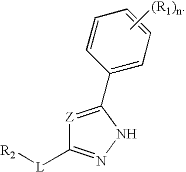

3. The compound of claim 1, wherein the compound comprises

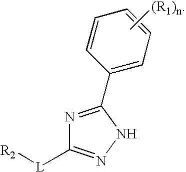

4. The compound of claim 1, wherein the compound comprises

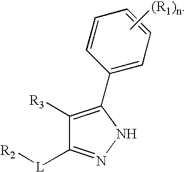

5. The compound of claim 1, wherein the compound comprises

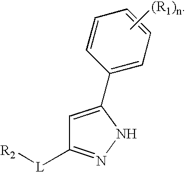

6. The compound of claim 1, wherein the compound comprises

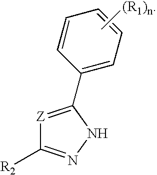

7. The compound of claim 1, wherein the compound comprises

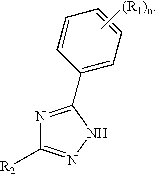

8. The compound of claim 1, wherein the compound comprises

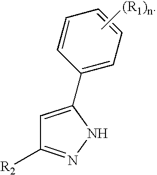

9. The compound of claim 1, wherein the compound comprises 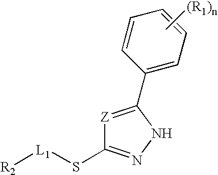
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

10. The compound according to claim 9, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

11. The compound according to claim 9, wherein L₁ is —O—.

12. The compound of claim 1, wherein the compound comprises 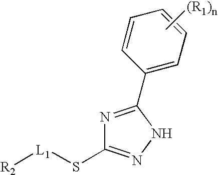
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

13. The compound of claim 1, wherein the compound comprises: 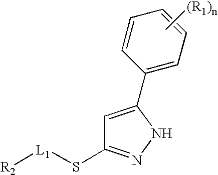
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

14. The compound of claim 1, wherein the compound comprises 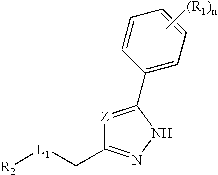
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

15. The compound according to claim 14, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

16. The compound according to claim 14, wherein L₁ is —O—.

17. The compound of claim 1, wherein the compound comprises 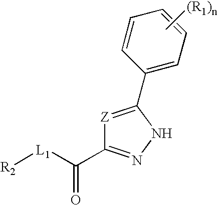
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

18. The compound according to claim 17, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

19. The compound according to claim 17, wherein L₁ is —O—.

20. The compound of claim 1, wherein the compound comprises 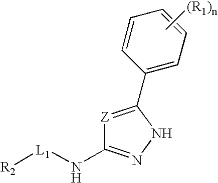
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

21. The compound according to claim 20, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

22. The compound according to claim 20, wherein L₁ is —O—.

23. The compound of claim 1, wherein the compound comprises 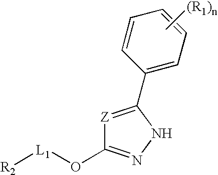
wherein L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

24. The compound according to claim 23, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

25. The compound according to claim 23, wherein L₁ is —O—.

26. The compound of claim 1, wherein the compound comprises: 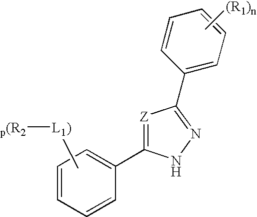
wherein
p is selected from the group consisting of 0, 1, 2, 3, 4 and 5; and
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur.

27. The compound according to claim 26, wherein p is selected from the group consisting of 1, 2, 3, 4 and 5.

28. The compound according to claim 26, wherein p is 2.

29. The compound according to claim 26, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

30. The compound according to claim 26, wherein L₁ is —O—.

31. The compound of claim 1, wherein the compound comprises 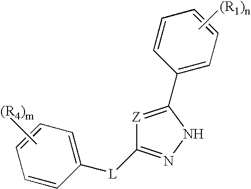
wherein
m is selected from the group consisting of 0, 1, 2, 3, 4 and 5; and
R₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₄ are taken together to form a ring.

32. The compound according to claim 31, wherein m is 2.

33. The compound according to claim 31, wherein R₄ is selected from the group consisting of halo, nitro, cyano, hydroxy, alkoxy, carbonyl, (C₁₋₁₀)alkyl and halo(C₁₋₁₀)alkyl, each substituted or unsubstituted, or two R₄ are taken together to form a ring.

34. The compound of claim 1, wherein the compound comprises 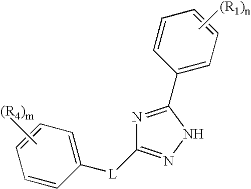
wherein
m is selected from the group consisting of 0, 1, 2, 3, 4 and 5; and
R₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₄ are taken together to form a ring.

35. The compound of claim 1, wherein the compound comprises 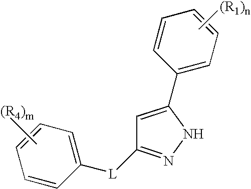
wherein
m is selected from the group consisting of 0, 1, 2, 3, 4 and 5; and
R₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₄ are taken together to form a ring.

36. The compound of claim 1, wherein the compound comprises 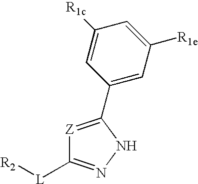
wherein R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

37. The compound according to claim 36, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

38. The compound according to claim 36, wherein R_{1c} and R₁ₑ are each independently a substituted or unsubstituted (C₁₋₅)alkoxy.

39. The compound according to claim 36, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

40. The compound of claim 1, wherein the compound comprises 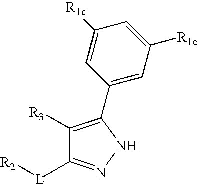
wherein R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

41. The compound according to claim 40, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

42. The compound according to claim 40, wherein R_{1c} and R₁ₑ are each independently a substituted or unsubstituted (C₁₋₅)alkoxy.

43. The compound according to claim 40, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

44. The compound of claim 1, wherein the compound comprises 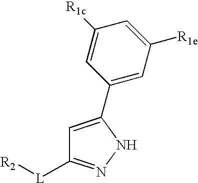
wherein R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

45. The compound according to claim 44, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

46. The compound according to claim 44, wherein R_{1c} and R₁ₑ are each independently a substituted or unsubstituted (C₁₋₅)alkoxy.

47. The compound according to claim 44, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

48. The compound of claim 1, wherein the compound comprises 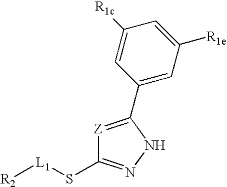
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

49. The compound according to claim 48, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

50. The compound according to claim 48, wherein L₁ is —O—.

51. The compound according to claim 48, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

52. The compound according to claim 48, wherein R_{1c} and R₁ₑ are each independently a substituted or unsubstituted (C₁₋₅)alkoxy.

53. The compound according to claim 48, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

54. The compound of claim 1, wherein the compound comprises 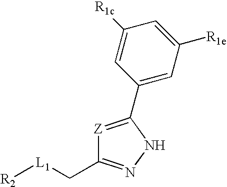
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

55. The compound according to claim 54, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

56. The compound according to claim 54, wherein L₁ is —O—.

57. The compound according to claim 54, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

58. The compound according to claim 54, wherein R_{1c} and R₁ₑ are each independently a substituted or unsubstituted (C₁₋₅)alkoxy.

59. The compound according to claim 54, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

60. The compound of claim 1, wherein the compound comprises 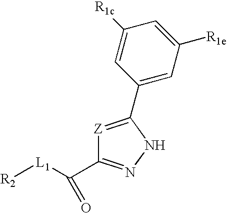
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

61. The compound according to claim 60, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

62. The compound according to claim 60, wherein L₁ is —O—.

63. The compound according to claim 60, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

64. The compound according to claim 60, wherein R_{1c} and R₁ₑ are each independently a substituted or unsubstituted (C₁₋₅)alkoxy.

65. The compound according to claim 60, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

66. The compound of claim 1, wherein the compound comprises 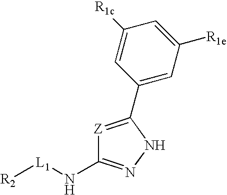
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

67. The compound according to claim 66, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

68. The compound according to claim 66, wherein L₁ is —O—.

69. The compound according to claim 66, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

70. The compound according to claim 66, wherein R_{1c} and R₁ₑ are each independently a substituted or unsubstituted (C₁₋₅)alkoxy.

71. The compound according to claim 66, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

72. The compound of claim 1, wherein the compound comprises 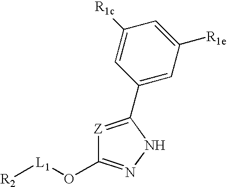
wherein
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur; and
R_{1c} and R₁ₑ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

73. The compound according to claim 72, wherein L₁ is absent or L₁ comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

74. The compound according to claim 72, wherein L₁ is —O—.

75. The compound according to claim 72, wherein R_{1c} and R₁ₑ are each independently selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

76. The compound according to claim 72, wherein R_{1c} and R₁ₑ are each independently a substituted or unsubstituted (C₁₋₅)alkoxy.

77. The compound according to claim 72, wherein
R_{1c} and R₁ₑ are each independently Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

78. The compound of claim 1, wherein the compound comprises 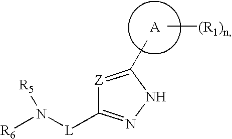
wherein R₅ and R₆ are each independently selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₅ and R₆ are taken together to form a substituted or unsubstituted ring.

79. The compound according to claim 78, wherein R₅ is H.

80. The compound according to claim 78, wherein R₆ is H.

81. The compound according to claim 1, wherein Z is CR₃.

82. The compound according to claim 1, wherein n is selected from the group consisting of 1, 2, 3, 4 and 5.

83. The compound according to claim 1, wherein n is 2.

84. The compound according to claim 1, wherein L is absent or L comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

85. The compound according to claim 1, wherein L is —O—.

86. The compound according to claim 1, wherein L is —O—(CH₂)ₘ—CO— and m is selected from the group consisting of 0, 1, 2, 3, 4, and 5.

87. The compound according to claim 1, wherein R₁ is selected from the group consisting of hydrogen, halo, nitro, oxy, hydroxy, alkoxy, aryloxy, heteroaryloxy, amino, (C₁₋₁₀)alkyl, and aryl(C₁₋₁₀)alkyl, each substituted or unsubstituted.

88. The compound according to claim 1, wherein at least one R₁ is alkoxy.

89. The compound according to claim 1, wherein at least one R₁ is methoxy.

90. The compound according to claim 1, wherein R₁ is 2-methoxy.

91. The compound according to claim 1, wherein
R₁ is Q-M-;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and
M is absent or a linker providing 1, 2, 3, 4, 5 or 6 atom separation between Q and the ring to which M is attached, wherein the atoms of the linker providing the separation are selected from the group consisting of carbon, oxygen, nitrogen, and sulfur.

92. The compound according to claim 91, wherein M is absent or M comprises a moiety selected from the group consisting of —CH₂—, —CH₂—O—, —CH═CH—, —CO—, —CO—O—, —CO—NH—, —NH—, —NH—CO—NH—, —O—, —O—CH₂—, —O—CH₂—CO—, —S—, —S—CH₂—, —S—CH₂—CO—, —S—CH₂—CO—NH—, -Ph-O—, -Ph-CO— and -Ph-CO—CH₂, each substituted or unsubstituted.

93. The compound according to claim 91, wherein M is —O—.

94. The compound according to claim 91, wherein M is selected from the group consisting of —CH₂—O— and —O—CH₂—.

95. The compound according to claim 91, wherein Q is a substituted or unsubstituted (C₄₋₁₂)aryl.

96. The compound according to claim 95, wherein Q is substituted with a substituent selected from the group consisting of (C₁₋₅)alkyl, sulfonyl and (C₁₋₅)alkylsulfonyl.

97. The compound according to claim 91, wherein Q is a substituted or unsubstituted phenyl.

98. The compound according to claim 97, wherein Q is substituted with a substituent selected from the group consisting of (C₁₋₅)alkyl, sulfonyl and (C₁₋₅)alkylsulfonyl.

99. The compound according to claim 91, wherein Q is a substituted or unsubstituted hetero(C₁₋₁₀)aryl.

100. The compound according to claim 99, wherein Q is substituted with a substituent selected from the group consisting of (C₁₋₅)alkyl, sulfonyl and (C₁₋₅)alkylsulfonyl.

101. The compound according to claim 91, wherein Q is a substituted or unsubstituted pyridinyl.

102. The compound according to claim 101, wherein Q is substituted with a substituent selected from the group consisting of (C₁₋₅)alkyl, sulfonyl and (C₁₋₅)alkylsulfonyl.

103. The compound according to claim 1, wherein R₂ is selected from the group consisting of hydrogen, hydroxy, carbonyl, amino, (C₁₋₁₀)alkyl, (C₃₋₁₂)cycloalkyl, heteron (C₃₋₁₂)alkyl, aryl and heteroaryl, each substituted or unsubstituted.

104. The compound according to claim 1, wherein R₂ is a substituted or unsubstituted (C₁₋₅)alkyl.

105. The compound according to claim 1, wherein R₂ is methyl.

106. The compound according to claim 1, wherein
R₂ is NR₅R₆; and
R₅ and R₆ are each independently selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₅ and R₆ are taken together to form a substituted or unsubstituted ring.

107. The compound according to claim 106, wherein R₅ is H.

108. The compound according to claim 106, wherein R₆ is H.

109. The compound according to claim 1, wherein R₃ is H.

110. The compound according to claim 1, wherein the compound is in the form of a pharmaceutically acceptable salt.

111. A compound selected from the group consisting of:
5-(2-methoxyphenyl)-3-(methylthio)-1H-1,2,4-triazole;
5-(2-ethoxyphenyl)-3-(2-fluorobenzylthio)-1H-1,2,4-triazole;
5-(2-(benzyloxy)phenyl)-3-(2-fluorobenzylthio)-1H-1,2,4-triazole;
5-(2,4-dimethoxyphenyl)-3-(2-fluorobenzylthio)-1H-1,2,4-triazole;
5-(2,5-dimethoxyphenyl)-3-(2-fluorobenzylthio)-1H-1,2,4-triazole;
2-((5-(2-methoxyphenyl)-1H-1,2,4-triazol-3-ylthio)methyl)pyridine;
3-((5-(2-methoxyphenyl)-1H-1,2,4-triazol-3-ylthio)methyl)pyridine;
2-(5-(2-methoxyphenyl)-1H-1,2,4-triazol-3-ylthio)-N,N-dimethylacetamide;
2-(5-(2-isopropoxyphenyl)-1H-1,2,4-triazol-3-ylthio)-1-morpholinoethanone;
2-(5-(2-isopropoxyphenyl)-1H-1,2,4-triazol-3-ylthio)-N,N-dimethylacetamide;
3-(2-fluorobenzyloxy)-5-(2-methoxyphenyl)-1H-pyrazole;
3-(2-fluorobenzyloxy)-5-phenyl-1H-pyrazole;
3-(2-fluorobenzyloxy)-5-(2-fluorophenyl)-1H-pyrazole;
5-(3,5-dimethoxyphenyl)-3-(2-fluorobenzyloxy)-1H-pyrazole;
3-(2-fluorobenzyloxy)-5-o-tolyl-1H-pyrazole;
5-(2,4-dimethoxyphenyl)-3-(2-fluorobenzyloxy)-1H-pyrazole;
3-((2-fluorophenoxy)methyl)-5-(2-methoxyphenyl)-1H-pyrazole;
5-(2-methoxyphenyl)-3-phenyl-1H-pyrazole;
3-cyclopropyl-5-(2-methoxyphenyl)-1H-pyrazole;
3-(4-chlorophenyl)-5-(2-methoxyphenyl)-1H-pyrazole;
2-(3-phenyl-1H-pyrazol-5-yl)phenol;
4-bromo-2-(3-phenyl-1H-pyrazol-5-yl)phenol;
5-(2,3-dimethoxyphenyl)-3-(2-fluorobenzyloxy)-1H-pyrazole;
5-(2,3-dihydrobenzofuran-7-yl)-3-(2-fluorobenzyloxy)-1H-pyrazole;
5-(5-(benzyloxy)-2-isopropoxyphenyl)-3-(2-fluorobenzyloxy)-1H-pyrazole;
5-(2,5-dimethoxyphenyl)-3-(2-fluorobenzyloxy)-1H-pyrazole;
5-(5-fluoro-2-methoxyphenyl)-3-(2-fluorobenzyloxy)-1H-pyrazole;
5-(2-(difluoromethoxy)phenyl)-3-(2-fluorobenzyloxy)-1H-pyrazole;
2-((2-(3-phenyl-1H-pyrazol-5-yl)phenoxy)methyl)pyridine;
(R)-6-((5-(2-methoxyphenyl)-1H-pyrazol-3-yloxy)methyl)piperazin-2-one;
2-(5-(2-methoxyphenyl)-1H-pyrazol-3-yloxy)acetic acid;
2-(5-(2-methoxyphenyl)-1H-pyrazol-3-yloxy)-1-morpholinoethanone;
(5-(2-methoxyphenyl)-1H-pyrazol-3-yl)(morpholino)methanone;
(5-(2,5-dimethoxyphenyl)-1H-pyrazol-3-yl)(morpholino)methanone;
5-(2-methoxyphenyl)-N-phenyl-1H-pyrazole-3-carboxamide;
3-(4-fluorophenyl)-5-(2-((1-methyl-1H-imidazol-2-yl)methoxy)phenyl)-1H-pyrazole;
(3-(5-(2-methoxyphenyl)-1H-pyrazol-3-yl)phenyl)(morpholino)methanone;
3-(5-(2-methoxyphenyl)-1H-pyrazol-3-yl)benzonitrile;
4-(5-(2-methoxyphenyl)-1H-pyrazol-3-yl)benzonitrile;
3-(4-fluorophenyl)-5-(2-phenoxyphenyl)-1H-pyrazole;
3-(4-fluorophenyl)-5-(3-phenoxyphenyl)-1H-pyrazole;
2-(3-(3-(4-fluorophenyl)-1H-pyrazol-5-yl)phenoxy)pyrimidine;
5-(4-(benzyloxy)-2-methoxyphenyl)-3-(4-fluorophenyl)-1H-pyrazole;
2-(3-(5-fluoro-2-methoxyphenyl)-1H-pyrazol-5-yl)pyridine;
3-(3,5-difluoro-2-methoxyphenyl)-5-(4-ethynylphenyl)-1H-pyrazole;
2-(3-(3-fluoro-2-methoxyphenyl)-1H-pyrazol-5-yl)pyridine;
3-(5-fluoro-2-methoxyphenyl)-5-(3-fluorophenyl)-1H-pyrazole;
3-(3-fluoro-2-methoxyphenyl)-5-(3-fluorophenyl)-1H-pyrazole;
5-(3-fluorophenyl)-3-(2-methoxyphenyl)-1H-pyrazole;
5-(4-fluorophenyl)-3-(1-methyl-1H-imidazol-2-yl)-1H-pyrazole;
3-benzyl-5-(2-methoxyphenyl)-4H-1,2,4-triazole;
3-benzyl-5-(4-tert-butylphenyl)-4H-1,2,4-triazole;
N-cyclopentyl-2-(5-methyl-4H-1,2,4-triazol-3-yl)aniline;
3-(2-(isopentyloxy)phenyl)-5-methyl-4H-1,2,4-triazole;
3-(2-(isopentyloxy)phenyl)-5-propyl-4H-1,2,4-triazole;
3-benzyl-5-(2-(isopentyloxy)phenyl)-4H-1,2,4-triazole;
3-benzyl-5-(2-isopropoxyphenyl)-4H-1,2,4-triazole;
5-(2-methoxyphenyl)-3-(trifluoromethyl)-1H-pyrazole;
5-(5-bromo-2-isobutoxyphenyl)-1H-pyrazole;
(E)-5-(2-isobutoxy-5-styrylphenyl)-1H-pyrazole;
5-(2-isobutoxy-5-phenethylphenyl)-1H-pyrazole;
5-(5-(benzyloxy)-2-(1-methoxypropan-2-yloxy)phenyl)-1H-pyrazol-3-ol;
5-(3-(2-fluorobenzyloxy)-5-(4-(methylsulfonyl)phenoxy)phenyl)-1H-pyrazol-3-ol;
(S)-5-(3-(2-fluorobenzyloxy)-5-(1-methoxypropan-2-yloxy)phenyl)-1H-pyrazol-3-ol;
(S)-5-(3-(1-methoxypropan-2-yloxy)-5-(4-(methylsulfonyl)phenoxy)phenyl)-1H-pyrazol-3-ol;
5-(3-(cyclopentyloxy)-5-(4-(methylsulfonyl)phenoxy)phenyl)-1H-pyrazol-3-ol;
5-(3-((3-methylpyridin-2-yl)methoxy)-5-(4-(methylsulfonyl)phenoxy)phenyl)-1H-pyrazol-3-ol;
5-(3-(2-methylbenzyloxy)-5-(4-(methylsulfonyl)phenoxy)phenyl)-1H-pyrazol-3-ol;
5-(5-(benzyloxy)-2-(1-methoxypropan-2-yloxy)phenyl)-3-methoxy-1H-pyrazole;
(S)-5-(3-(2-fluorobenzyloxy)-5-(1-methoxypropan-2-yloxy)phenyl)-3-methoxy-1H -pyrazole;
5-(3-(2-fluorobenzyloxy)-5-(4-(methylsulfonyl)phenoxy)phenyl)-3-methoxy-1H-pyrazole;
5-(3-(cyclopentyloxy)-5-(4-(methylsulfonyl)phenoxy)phenyl)-3-methoxy-1H-pyrazole;
2-((3-(3-methoxy-1H-pyrazol-5-yl)-5-(4-(methylsulfonyl)phenoxy)phenoxy)methyl)-3-methylpyridine;
3-methoxy-5-(3-(2-methylbenzyloxy)-5-(4-(methylsulfonyl)phenoxy)phenyl)-1H-pyrazole;
5-(2-(5-(5-(benzyloxy)-2-(1-methoxypropan-2-yloxy)phenyl)-1H-pyrazol-3-yloxy)ethyl)-4-methylthiazole;
(S)-5-(2-(5-(3-(2-fluorobenzyloxy)-5-(1-methoxypropan-2-yloxy)phenyl)-1H-pyrazol-3-yloxy)ethyl)-4-methylthiazole;
(S)-methyl 2-(5-(3-(1-methoxypropan-2-yloxy)-5-(4-(methylsulfonyl)phenoxy)phenyl)-1H-pyrazol-3-yloxy)acetate;
3-(3-methoxy-1H-pyrazol-5-yl)-4-(1-methoxypropan-2-yloxy)phenol;
3-(3-methoxy-1H-pyrazol-5-yl)-5-(4-(methylsulfonyl)phenoxy)phenol;
(S)-3-(3-methoxy-1H-pyrazol-5-yl)-5-(1-methoxypropan-2-yloxy)phenol;
5-(5-ethoxy-2-(1-methoxypropan-2-yloxy)phenyl)-3-methoxy-1H-pyrazole;
3-methoxy-5-(2-(1-methoxypropan-2-yloxy)-5-(2-(thiophen-3-yl)ethoxy)phenyl)-1H -pyrazole;
3-methoxy-5-(2-(1-methoxypropan-2-yloxy)-5-(4-(methylsulfonyl)benzyloxy)phenyl)-1H-pyrazole;
4-((3-(3-methoxy-1H-pyrazol-5-yl)-4-(1-methoxypropan-2-yloxy)phenoxy)methyl)pyridine;
(S)-3-methoxy-5-(3-(1-methoxypropan-2-yloxy)-5-(4-(methylsulfonyl)phenoxy)phenyl)-1H-pyrazole;
(S)-3-methoxy-5-(3-(1-methoxypropan-2-yloxy)-5-(4-(methylsulfonyl)benzyloxy)phenyl)-1H-pyrazole;
(S)-3-methoxy-5-(3-(1-methoxypropan-2-yloxy)-5-(2-(thiophen-3-yl)ethoxy)phenyl)-1H-pyrazole;
(S)-2-(5-(3-(1-methoxypropan-2-yloxy)-5-(4-(methylsulfonyl)phenoxy)phenyl)-1H -pyrazol-3-yloxy)acetic acid;
(S)-1-(4-acetylpiperazin-1-yl)-2-(5-(3-(1-methoxypropan-2-yloxy)-5-(4-(methylsulfonyl)phenoxy)phenyl)-1H-pyrazol-3-yloxy)ethanone;
(S)-2-(5-(3-(1-methoxypropan-2-yloxy)-5-(4-(methylsulfonyl)phenoxy)phenyl)-1H -pyrazol-3-yloxy)-1-morpholinoethanone;
(S)—N-ethyl-2-(5-(3-(1-methoxypropan-2-yloxy)-5-(4-(methylsulfonyl)phenoxy)phenyl)-1H-pyrazol-3-yloxy)acetamide;
1-isobutyl-6-(1H-pyrazol-5-yl)-1H-benzo [d]imidazole;
1-(cyclohexylmethyl)-6-(1H-pyrazol-5-yl)-1H-benzo[d]imidazole;
5-(1-isobutyl-1H-benzo[d]imidazol-6-yl)-1H-pyrazol-3-ol;
5-(1-(cyclohexylmethyl)-1H-benzo [d]imidazol-6-yl)-1H-pyrazol-3-ol;
1-isobutyl-6-(3-methoxy-1H-pyrazol-5-yl)-1H-benzo[d]imidazole;
1-(cyclohexylmethyl)-6-(3-methoxy-1H-pyrazol-5-yl)-1H-benzo[d]imidazole;
5-benzyl-3-phenyl-1H-1,2,4-triazole;
2-(3-phenyl-1H-1,2,4-triazol-5-yl)phenol;
(3-(2-ethoxyphenyl)-1H-1,2,4-triazol-5-yl)methanol;
1-(4-(methylthio)phenyl)-3-(5-phenyl-1H-1,2,4-triazol-3-yl)urea;
1-(5-phenyl-1H-1,2,4-triazol-3-yl)-3-(2-(trifluoromethyl)phenyl)urea;
1-(2,5-difluorophenyl)-3-(5-phenyl-1H-1,2,4-triazol-3-yl)urea;
5-(3,4,5-trimethoxyphenyl)-1H-1,2,4-triazol-3-amine;
N-(5-bromopyridin-2-yl)-2-(3-phenyl-1H-1,2,4-triazol-5-ylthio)acetamide;
2,2′-(1H-1,2,4-triazole-3,5-diyl)diphenol;
1-(3,4-dihydroxyphenyl)-2-(5-(2-methoxyphenyl)-1H-1,2,4-triazol-3-ylthio)ethanone;
2-(5-(2-chlorophenyl)-1H-1,2,4-triazol-3-ylthio)-N-cyclohexylacetamide;
(E)-2-(3-(4-bromostyryl)-1H-1,2,4-triazol-5-yl)phenol;
(E)-2-(3-(2-chlorostyryl)-1H-1,2,4-triazol-5-yl)phenol;
(E)-2-(3-(2-(3-methylthiophen-2-yl)vinyl)-1H-1,2,4-triazol-5-yl)phenol;
(E)-5-(2-(5-(2-hydroxyphenyl)-1H-1,2,4-triazol-3-yl)vinyl)-2-methoxyphenol;
3-(2-fluorobenzylthio)-5-(2-methoxyphenyl)-1H-1,2,4-triazole;
N-(3,4-dimethylphenyl)-2-(5-(2-methoxyphenyl)-1H-1,2,4-triazol-3-ylthio)acetamide;
N-(5-(3,4-dimethoxyphenyl)-1H-1,2,4-triazol-3-yl)-4,6-dimethylpyrimidin-2-amine;
2-(3-phenyl-1H-1,2,4-triazol-5-ylthio)-N-p-tolylacetamide;
3-(4-chlorophenyl)-1H-1,2,4-triazol-5-amine;
N-phenyl-2-(3-phenyl-1H-1,2,4-triazol-5-ylthio)acetamide;
N-(2-nitrophenyl)-2-(3-phenyl-1H-1,2,4-triazol-5-ylthio)acetamide;
N-(3,5-dibromopyridin-2-yl)-2-(3-phenyl-1H-1,2,4-triazol-5-ylthio)acetamide;
3-(2,4-dinitrophenyl)-1H-1,2,4-triazol-5-amine;
3-(2-fluorophenyl)-1H-1,2,4-triazol-5-amine;
N-(2,4-dimethylphenyl)-2-(3-(3-nitrophenyl)-1H-1,2,4-triazol-5-ylthio)acetamide;
3-(2-methoxyphenyl)-1H-1,2,4-triazol-5-amine;
3-(3,5-dimethoxyphenyl)-1H-1,2,4-triazol-5-amine;
5-(1H-pyrrol-2-yl)-3-(thiophen-2-yl)-1H-1,2,4-triazole;
N-(5-(2-methoxyphenyl)-1H-1,2,4-triazol-3-yl)-4,6-dimethylpyrimidin-2-amine;
3-(3-bromobenzylthio)-5-(2-methoxyphenyl)-1H-1,2,4-triazole;
2-(5-(2-methoxyphenyl)-1H-1,2,4-triazol-3-ylthio)-1-morpholinoethanone;
4-((5-(2-methoxyphenyl)-1H-1,2,4-triazol-3-ylthio)methyl)benzoic acid;
2-(5-(2-methoxyphenyl)-1H-1,2,4-triazol-3-ylthio)-N-(2-nitrophenyl)acetamide;
3-benzyl-5-(2-methoxyphenyl)-1H-1,2,4-triazole;
5-benzyl-3-(4-tert-butylphenyl)-1H-1,2,4-triazole;
N-cyclopentyl-2-(5-methyl-1H-1,2,4-triazol-3-yl)aniline;
2-(5-(2-isopropoxyphenyl)-1H-1,2,4-triazol-3-ylthio)acetic acid;
2-(5-(2-isopropoxyphenyl)-1H-1,2,4-triazol-3-ylthio)acetamide;
4-bromo-2-(1H-pyrazol-5-yl)phenol;
5-methyl-3-phenyl-1H-pyrazole-4-carboxamide;
4-chloro-2-(3-methyl-1H-pyrazol-5-yl)aniline;
3,5-diphenyl-1H-pyrazole;
2,4-difluoro-6-(1H-pyrazol-5-yl)phenol;
3-(4-chlorophenyl)-5-(3-phenyl-1H-pyrazol-4-yl)-1,2,4-oxadiazole;
4-ethyl-6-(4-(thiazol-4-yl)-1H-pyrazol-3-yl)benzene-1,3-diol;
4-methyl-6-(4-(2-methylthiazol-4-yl)-1H-pyrazol-3-yl)benzene-1,3-diol;
5-methoxy-3-methyl-2-(5-methyl-4-phenoxy-1H-pyrazol-3-yl)phenol;
4-(4-(4-methoxyphenyl)-5-methyl-1H-pyrazol-3-yl)benzene-1,3-diol;
3-(4-chlorophenyl)-5-(trifluoromethyl)-1H-pyrazole;
3-phenyl-1H-pyrazol-5-ol;
3,5-diphenyl-1H-pyrazol-4-ol;
5-(pyridin-2-yl)-1H-pyrazole-3-carboxylic acid;
2-(3-phenyl-1H-pyrazol-5-yl)pyridine;
N-(3-acetamidophenyl)-5-(pyridin-2-yl)-1H-pyrazole-3-carboxamide;
N-phenyl-5-(pyridin-2-yl)-1H-pyrazole-3-carboxamide;
N-(3-nitrophenyl)-5-(pyridin-2-yl)-1H-pyrazole-3-carboxamide;
N-(5-methylthiazol-2-yl)-5-(pyridin-2-yl)-1H-pyrazole-3-carboxamide;
5-(2,5-bis(2,2,2-trifluoroethoxy)phenyl)-1H-pyrazole;
3-(5-bromo-2-isobutoxyphenyl)-1H-pyrazole;
2-(5-methyl-1H-pyrazol-3-yl)-1H-benzo[d]imidazole;
2-(5-methyl-1H-pyrazol-3-yl)-1H-benzo[d]imidazol-4-ol;
2-(5-methyl-1H-pyrazol-3-yl)benzo[d]oxazol-4-amine;
7-bromo-2-(5-methyl-1H-pyrazol-3-yl)-3H-imidazo[4,5-c]pyridine;
2-(3-methyl-1H-pyrazol-5-yl)-7-phenyl-3H-imidazo[4,5-c]pyridine;
7-(3-(ethylsulfonyl)phenyl)-2-(5-methyl-1H-pyrazol-3-yl)-3H-imidazo[4,5-c]pyridine;
7-(1-(ethylsulfonyl)-1H-indol-6-yl)-2-(5-methyl-1H-pyrazol-3-yl)-3H-imidazo[4,5-c]pyridine;
N-(4-(2-(5-methyl-1H-pyrazol-3-yl)-3H-imidazo[4,5-c]pyridin-7-ylthio)phenyl)acetamide;
(E)-3-(2-isobutoxy-5-styrylphenyl)-1H-pyrazole;
5-(2,5-dimethoxyphenyl)-1H-pyrazole-3-carboxylic acid;
ethyl 5-phenyl-1H-pyrazole-3-carboxylate;
3-(2-isobutoxy-5-phenethylphenyl)-1H-pyrazole;
ethyl 5-(2-methoxyphenyl)-1H-pyrazole-3-carboxylate;
(5-(2-methoxyphenyl)-1H-pyrazol-3-yl)methanol;
2-(5-(2-methoxyphenyl)-1H-pyrazol-3-yloxy)acetic acid;
5-(3-fluorophenyl)-3-(1-methyl-1H-imidazol-2-yl)-1H-pyrazole;
3-(3,5-difluoro-2-methoxyphenyl)-5-(3-fluorophenyl)-1H-pyrazole;
2-(3-(3,5-difluoro-2-methoxyphenyl)-1H-pyrazol-5-yl)pyridine;
3,5-di(pyridin-2-yl)-1H-pyrazole;
ethyl 2-(5-(5-chloro-2-methoxyphenyl)-1H-1,2,4-triazol-3-ylthio)acetylcarbamate;
(R)-tert-butyl 3-((5-(2-methoxyphenyl)-1H-pyrazol-3-yloxy)methyl)-5-oxopiperazine-1-carboxylate;
3-(4-fluorophenyl)-5-(2-methoxyphenyl)-1H-pyrazole;
5-(2,3-dihydrobenzofuran-7-yl)-3-phenyl-1H-pyrazole;
5-(2,2-dimethyl-2,3-dihydrobenzofuran-7-yl)-3-phenyl-1H-pyrazole;
5-(5-bromo-2,3-dihydrobenzofuran-7-yl)-3-phenyl-1H-pyrazole;
4-(3-phenyl-1H-pyrazol-5-yl)benzo [c][1,2,5]thiadiazole;
2-(5-(2-isobutoxyphenyl)-1H-pyrazol-3-yl)pyridine;
3-(5-(2-isobutoxyphenyl)-1H-pyrazol-3-yl)pyridine;
2-(5-(5-chloro-2-isobutoxyphenyl)-1H-pyrazol-3-yl)pyridine;
3-(5-(5-chloro-2-isobutoxyphenyl)-1H-pyrazol-3-yl)pyridine;
5-(2-methoxy-5-nitrophenyl)-3-phenyl-1H-pyrazole;
3-(5-(2-methoxyphenyl)-1H-pyrazol-3-yl)aniline;
5-(4-(benzyloxy)-2-(2-methoxyethoxy)phenyl)-3-phenyl-1H-pyrazole;
3-(5-(2-methoxyphenyl)-1H-pyrazol-3-yl)pyridine;
4-(5-(5-(2-methoxyphenyl)-1H-pyrazol-3-yl)pyridin-2-yl)morpholine;
5-(1-benzyl-1H-imidazol-2-yl)-3-(3-fluorophenyl)-1H-pyrazole;
3-(5-(1-benzyl-1H-imidazol-2-yl)-1H-pyrazol-3-yl)pyridine;
2-(2-(3-(3-fluorophenyl)-1H-pyrazol-5-yl)phenoxy)-N,N-dimethylacetamide;
N,N-dimethyl-2-(2-(3-(pyridin-3-yl)-1H-pyrazol-5-yl)phenoxy)acetamide;
2-((5-(benzyloxy)-2-(3-phenyl-1H-pyrazol-5-yl)phenoxy)methyl)pyridine;
N-(3-(5-(2-methoxyphenyl)-1H-pyrazol-3-yl)phenyl)acetamide;
N-(3-(5-(2-methoxyphenyl)-1H-pyrazol-3-yl)phenyl)benzamide;
2-(2-(5-(4-cyanophenyl)-1H-pyrazol-3-yl)phenoxy)-N,N-dimethylacetamide;
2-(2-(5-(pyridin-3-yl)-1H-pyrazol-3-yl)phenoxy)-1-(pyrrolidin-1-yl)ethanone;
5-(5-(2-methoxyphenyl)-1H-pyrazol-3-yl)-N,N-dimethylpyridin-2-amine;
5-(3-(3-fluorophenyl)-1H-pyrazol-5-yl)-2,4-dimethoxypyrimidine;
2,4-dimethoxy-5-(3-(pyridin-3-yl)-1H-pyrazol-5-yl)pyrimidine;
3-(2-(3-phenyl-1H-pyrazol-5-yl)phenoxy)pyridine;
2-(3-(3-phenyl-1H-pyrazol-5-yl)phenoxy)pyridine;
5-(2-methoxy-4-nitrophenyl)-3-phenyl-1H-pyrazole;
4-(5-(2-methoxyphenyl)-1H-pyrazol-3-yl)aniline;
3-methoxy-4-(3-phenyl-1H-pyrazol-5-yl)aniline;
5-(2-methoxy-4-(4-methoxybenzyloxy)phenyl)-3-phenyl-1H-pyrazole;
N-(3-methoxy-4-(3-phenyl-1H-pyrazol-5-yl)phenyl)benzamide;
N-(3-methoxy-4-(3-phenyl-1H-pyrazol-5-yl)phenyl)-2-phenylacetamide;
2-(2-(3-(3-fluorophenyl)-1H-pyrazol-5-yl)phenoxy)-1-(pyrrolidin-1-yl)ethanone;
4-(5-(2-(2-oxo-2-(pyrrolidin-1-yl)ethoxy)phenyl)-1H-pyrazol-3-yl)benzonitrile;
2-(2-(3-(3-fluorophenyl)-1H-pyrazol-5-yl)phenoxy)-N,N-dimethylethanamine;
N-(2,4-dimethoxybenzyl)-5-(5-(2-methoxyphenyl)-1H-pyrazol-3-yl)pyridin-2-amine;
5-(5-(2-methoxyphenyl)-1H-pyrazol-3-yl)pyridin-2-amine;
3-(2-chlorophenyl)-5-(2-methoxyphenyl)-1H-pyrazole;
5-(2-methoxyphenyl)-3-o-tolyl-1H-pyrazole;
3-(2-chloro-6-fluorophenyl)-5-(2-methoxyphenyl)-1H-pyrazole;
N-(2-methoxyethyl)-5-(5-(2-methoxyphenyl)-1H-pyrazol-3-yl)pyridin-2-amine;
3-(5-(2-(3-chlorophenoxy)phenyl)-1H-pyrazol-3-yl)pyridine;
2-(2-(3-(pyridin-3-yl)-1H-pyrazol-5-yl)phenoxy)benzonitrile;
5-(6-methoxybenzofuran-5-yl)-3-phenyl-1H-pyrazole;
5-(2-methoxyphenyl)-1H-pyrazole-3-carboxylic acid;
5-(2-methoxyphenyl)-N-(3-methoxyphenyl)-1H-pyrazole-3-carboxamide;
1-((5-(2-methoxyphenyl)-1H-pyrazol-3-yl)methyl)-1H-benzo[d]imidazole;
3-((5-(2-methoxyphenyl)-1H-pyrazol-3-yl)methyl)quinazoline-2,4(1H,3H)-dione;
(5-(4-(benzyloxy)-2-methoxyphenyl)-1H-pyrazol-3-yl)methanamine;
6-methoxy-3-((5-(2-methoxyphenyl)-1H-pyrazol-3-yl)methyl)quinazolin-4(3H)-one;
6-((5-(4-(benzyloxy)-2-methoxyphenyl)-1H-pyrazol-3-yl)methylamino)-3-methylpyrimidine-2,4(1H,3H)-dione;
3-methoxy-N-((5-(2-methoxyphenyl)-1H-pyrazol-3-yl)methyl)aniline;
4-methoxy-3-(3-phenyl-1H-pyrazol-5-yl)aniline;
N-benzyl-4-methoxy-3-(3-phenyl-1H-pyrazol-5-yl)aniline;
N-benzyl-3-methoxy-4-(3-phenyl-1H-pyrazol-5-yl)aniline;
2-((5-(4-(benzyloxy)-2-methoxyphenyl)-1H-pyrazol-3-yl)methylamino)-6-fluoroquinazolin-4(3H)-one;
2-((5-(2-methoxyphenyl)-1H-pyrazol-3-yl)methyl)isoindoline-1,3-dione;
N-(4-methoxy-3-(3-phenyl-1H-pyrazol-5-yl)phenyl)benzamide;
N-(4-methoxy-3-(3-phenyl-1H-pyrazol-5-yl)phenyl)benzenesulfonamide;
N-(4-methoxy-3-(3-phenyl-1H-pyrazol-5-yl)phenyl)-2-phenylacetamide;
N-(4-methoxy-3-(3-phenyl-1H-pyrazol-5-yl)phenyl)isonicotinamide;
N-(4-methoxy-3-(3-phenyl-1H-pyrazol-5-yl)phenyl)thiazole-5-carboxamide;
N-(4-methoxy-3-(3-phenyl-1H-pyrazol-5-yl)phenyl)furan-3-carboxamide;
N-(4-methoxy-3-(3-phenyl-1H-pyrazol-5-yl)phenyl)tetrahydrofuran-3-carboxamide;
3-methoxy-5-(2-methoxyphenyl)-1H-pyrazole;
3-ethoxy-5-(2-methoxyphenyl)-1H-pyrazole;
5-(2-methoxyphenyl)-3-propoxy-1H-pyrazole;
3-isopropoxy-5-(2-methoxyphenyl)-1H-pyrazole;
3-sec-butoxy-5-(2-methoxyphenyl)-1H-pyrazole;
3-(cyclopentyloxy)-5-(2-methoxyphenyl)-1H-pyrazole;
5-(2-methoxyphenyl)-3-((tetrahydrofuran-2-yl)methoxy)-1H-pyrazole;
3-(2-methoxyethoxy)-5-(2-methoxyphenyl)-1H-pyrazole;
5-(2-methoxyphenyl)-3-(3-methoxypropoxy)-1H-pyrazole;
2-(5-(2-methoxyphenyl)-1H-pyrazol-3-yloxy)-N,N-dimethylethanamine;
3-(5-(2-methoxyphenyl)-1H-pyrazol-3-yloxy)-N,N-dimethylpropan-1-amine;
3-(benzyloxy)-5-(2-methoxyphenyl)-1H-pyrazole;
5-(2-methoxyphenyl)-3-phenethoxy-1H-pyrazole;
3-(3-fluorobenzyloxy)-5-(2-methoxyphenyl)-1H-pyrazole;
3-(4-fluorobenzyloxy)-5-(2-methoxyphenyl)-1H-pyrazole;
4-((5-(2-methoxyphenyl)-1H-pyrazol-3-yloxy)methyl)benzonitrile;
3-(2,4-difluorobenzyloxy)-5-(2-methoxyphenyl)-1H-pyrazole;
2-((5-(2-methoxyphenyl)-1H-pyrazol-3-yloxy)methyl)pyridine;
4-((5-(2-methoxyphenyl)-1H-pyrazol-3-yloxy)methyl)pyridine;
5-(2-methoxyphenyl)-3-(thiophen-2-ylmethoxy)-1H-pyrazole;
(S)-5-(2-methoxyphenyl)-3-(1-phenylethoxy)-1H-pyrazole;
N,N-diethyl-2-(5-(2-methoxyphenyl)-1H-pyrazol-3-yloxy)ethanamine;
5-(2-methoxyphenyl)-3-(2-(thiophen-3-yl)ethoxy)-1H-pyrazole;
1-(3-(5-(2-methoxyphenyl)-1H-pyrazol-3-yloxy)propyl)pyrrolidin-2-one;
1-(2-(5-(2-methoxyphenyl)-1H-pyrazol-3-yloxy)ethyl)imidazolidin-2-one;
2-(5-(2-methoxyphenyl)-1H-pyrazol-3-yloxy)-1-phenylethanone;
5-(2-(5-(2-methoxyphenyl)-1H-pyrazol-3-yloxy)ethyl)-4-methylthiazole;
2-((5-(2-methoxyphenyl)-1H-pyrazol-3-yloxy)methyl)thiazole;
5-(2-methoxyphenyl)-3-((1-methyl-1H-imidazol-5-yl)methoxy)-1H-pyrazole; and
5-(2-methoxyphenyl)-3-((1-methyl-1H-imidazol-2-yl)methoxy)-1H-pyrazole.

112. The compound according to claim 111, wherein the compound is in the form of a pharmaceutically acceptable salt.

113. A pharmaceutical composition comprising as an active ingredient a compound according to claim 1.

114. A pharmaceutical composition comprising a compound according to claim 1, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

115. A kit comprising:
a compound of claim 1; and
instructions which comprise one or more forms of information selected from the group consisting of indicating a disease state for which the compound is to be administered, storage information for the compound, dosing information and instructions regarding how to administer the compound.

116. An article of manufacture comprising:
a compound of claim 1; and
packaging materials.

117. A therapeutic method comprising administering a compound of claim 1 to a subject.

118. A method of activating glucokinase comprising contacting glucokinase with a compound of claim 1.

119. A method of activating glucokinase comprising causing a compound of claim 1 to be present in a subject in order to activate glucokinase in vivo.

120. A method of activating glucokinase comprising administering a first compound to a subject that is converted in vivo to a second compound wherein the second compound activates glucokinase in vivo, the second compound being a compound according to claim 1.

121. A method of treating a disease state for which increasing glucokinase activity ameliorates the pathology and/or symptomology of the disease state, the method comprising causing a compound of claim 1 to be present in a subject in a therapeutically effective amount for the disease state.

122. A method of treating a disease state for which increasing glucokinase activity ameliorates the pathology and/or symptomology of the disease state, the method comprising administering a compound of claim 1 to a subject, wherein the compound is present in the subject in a therapeutically effective amount for the disease state.

123. A method of treating a disease state for which increasing glucokinase activity ameliorates the pathology and/or symptomology of the disease state, the method comprising administering a first compound to a subject that is converted in vivo to a second compound wherein the second compound activates glucokinase in vivo, the second compound being a compound according to claim 1.

124. A process comprising:
reacting a compound comprising the formula 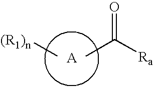
with hydrazinecarbothioamide under conditions that form a first reaction product comprising the formula 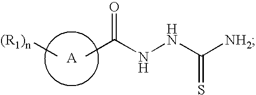
treating the first reaction product under conditions that form a second reaction product comprising the formula 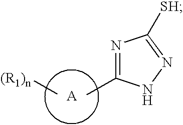
and
reacting the second reaction product with a compound comprising the formula R₂-L₁-X under conditions that form a product comprising the formula 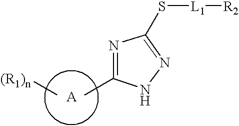
wherein
X and Rₐ are each independently a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

125. A process comprising:
treating a compound comprising the formula 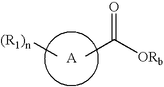
under conditions that form a first reaction product comprising the formula 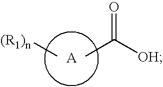
reacting the first reaction product with a compound comprising the formula R_{c}O₂C—C(R₃)H—CO₂K under conditions that form a second reaction product comprising the formula 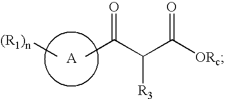
treating the second reaction product under conditions that form a third reaction product comprising the formula 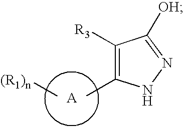
and
reacting the third reaction product with a compound comprising the formula R₂-L₁-OH under conditions that form a product comprising the formula 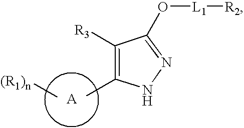
wherein
R_{b} and R_{c} are each independently a (C₁₋₅)alkyl;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

126. A process comprising:
reacting a compound comprising the formula 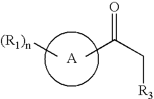
with a compound comprising the formula (CO₂R_{d})₂ under conditions that form a first reaction product comprising the formula 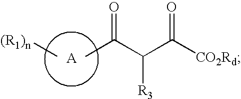
treating the first reaction product under conditions that form a second reaction product comprising the formula 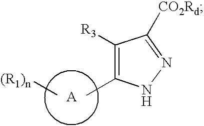
treating the second reaction product under conditions that form a third reaction product comprising the formula 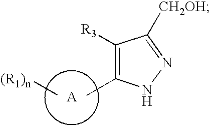
and
reacting the third reaction product with a compound comprising the formula R₂—OH under conditions that form a product comprising the formula 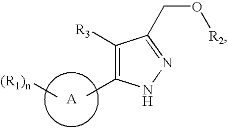
wherein
R_{d} is a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

127. A process comprising:
reacting a compound comprising the formula 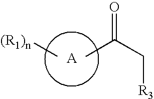
with a compound comprising the formula R₂-L-CO₂Rₑ under conditions that form a first reaction product comprising the formula 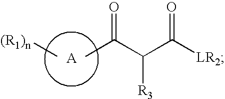
and
treating the first reaction product under conditions that form a product comprising the formula 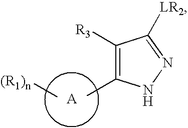
wherein
Rₑ is a (C₁₋₅)alkyl;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

128. A process comprising:
reacting a compound comprising the formula 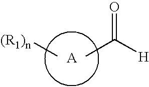
with a compound comprising the formula
R₃ [symbol] LR₂
under conditions that form a product comprising the formula 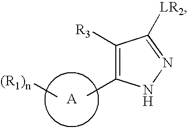
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

129. A process comprising:
reacting a compound comprising the formula 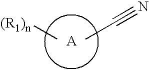
with a compound comprising the formula 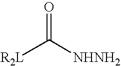
under conditions that form a product comprising the formula 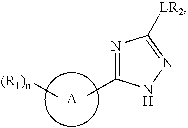
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and

130. A process comprising:
treating a compound comprising the formula 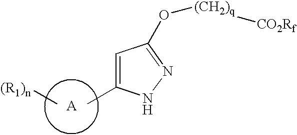
under conditions that form a first reaction product comprising the formula 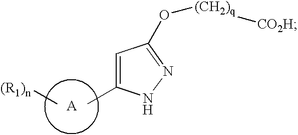
reacting the first reaction product with a compound comprising the formula HNR₅R₆ under conditions that form a product comprising the formula 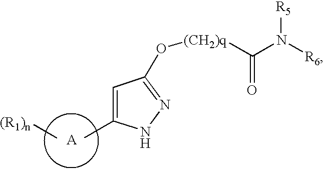
wherein
n is selected from the group consisting of 0, 1, 2, 3, 4 and 5;
q is selected from the group consisting of 0, 1, 2, 3, 4 and 5;
R_{f} is a (C₁₋₅)alkyl;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₅ and R₆ are each independently selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₅ and R₆ are taken together to form a substituted or unsubstituted ring.

131. A process comprising:
treating a compound comprising the formula 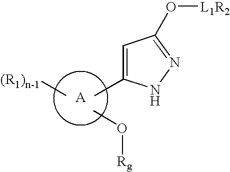
under conditions that form a first reaction product comprising the formula 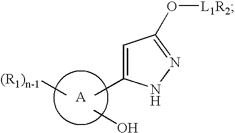
and
reacting the first reaction product with a compound comprising Q-OH under conditions that form a product comprising the formula 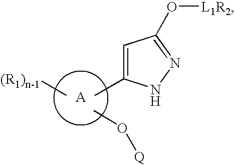
wherein
R_{g} is a leaving group;
Q is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

132. A process comprising:
reacting a compound comprising the formula 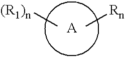
with a compound comprising the formula 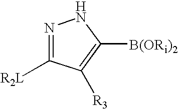
under conditions that form a product comprising the formula 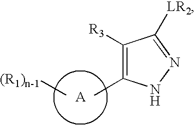
wherein
Rₕ and Rᵢ are each independently a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

133. A compound comprising the formula 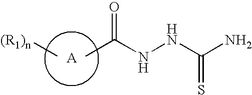
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

134. A compound comprising the formula 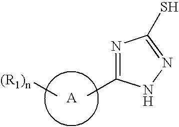
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

135. A compound comprising the formula 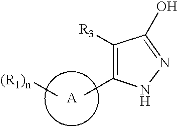
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

136. A compound comprising the formula 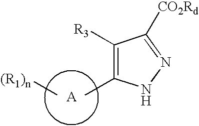
wherein
R_{d} is a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

137. A compound comprising the formula 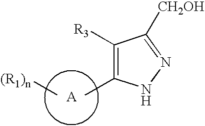
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

138. A compound comprising the formula 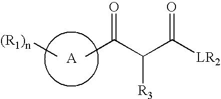
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

139. A compound comprising the formula 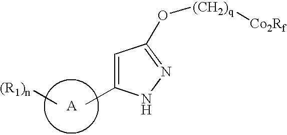
wherein
R_{f} is a (C₁₋₅)alkyl;
n is selected from the group consisting of 0, 1, 2, 3, 4 and 5;
q is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

140. A compound comprising the formula 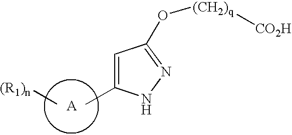
wherein
n is selected from the group consisting of 0, 1, 2, 3, 4 and 5;
q is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring.

141. A compound comprising the formula 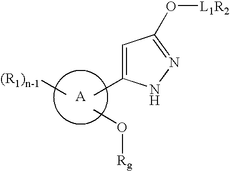
wherein
R_{g} is a leaving group;
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

142. A compound comprising the formula 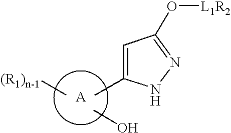
wherein
n is selected from the group consisting of 0, 1, 2, 3, 4 and 5;
A is a ring selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
L₁ is absent or a linker providing 1, 2, 3, 4 or 5 atom separation between R₂ and the atom to which L₁ is attached, wherein the atoms of the linker providing the separation are selected from the group consisting of carbon, oxygen, nitrogen, and sulfur;
R₁ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or two R₁ are taken together to form a ring; and
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

143. A compound comprising the formula 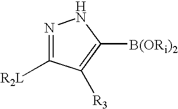
wherein
R₁ is a leaving group;
L is absent or a linker providing 1, 2, 3, 4, 5 or 6 atom separation between R₂ and the ring to which L is attached, wherein the atoms of the linker providing the separation are selected from the group consisting of carbon, oxygen, nitrogen, and sulfur;
R₂ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and
R₃ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.
